(19) United States
(12) Patent Application Publication
(10) Pub. No.: US 2012/0015901 A1
(43) Pub. Date: Jan. 19, 2012

(54) METHODS AND PREPARATIONS FOR PROTECTING CRITICALLY ILL PATIENTS

(75) Inventors: Joris Winderickx, Wilsele (BE); Greet Van Den Berghe, Grez-Doiceau (BE); Jan Gunst, Ieper (BE); IIse Vanhorebeek, Linden (BE); Lies Langouche, Leuven (BE); Tobias Eisenberg, Graz (AT); Frank Madeo, Graz (AT); Christophe Magnes, Kumberg (AT); Frank Sinner, Graz (AT)

(73) Assignees: Katholieke Universiteit Leuven, K.U.Leuven R&D, Leuven (BE); Joanneum Research Forschungsgesellschaft mbH, Graz (AT); University of Graz, Graz (AT)

(21) Appl. No.: 13/181,698

(22) Filed: Jul. 13, 2011

Related U.S. Application Data
(63) Continuation-in-part of application No. PCT/EP2010/050426, filed on Jan. 14, 2010.
(60) Provisional application No. 61/363,852, filed on Jul. 13, 2010.

(30) Foreign Application Priority Data
Jan. 14, 2009 (GB) 0900514.1
Jan. 15, 2009 (NL) 1036427
Jun. 9, 2009 (GB) 0909894.8
Jun. 11, 2009 (GB) 0910048.8
Nov. 5, 2009 (GB) 0919448.1
Nov. 24, 2009 (GB) 0920456.1

(51) Int. Cl. A61K 31/132; A61K 31/575; A61K 31/16; A61K 31/436; A61K 31/7016; A61P 1/16; A61K 31/455; A61P 17/02; A61P 25/00; A61P 11/00; A61P 13/12; A61K 31/13; A61K 31/05

(52) U.S. Cl. 514/53; 514/579; 514/673; 514/674; 514/182; 514/626; 514/291; 514/733; 514/355

(57) ABSTRACT

The present invention relates to a method of treating a life threatening condition in a critically ill human patient with a non-infectuous disorder, wherein the critically ill patient is a patient receiving enteral or parenteral nutrition, the method comprising the step of administering to said patient an autophagy inducing agent.

### CROSS REFERENCE TO RELATED APPLICATIONS

This application is a continuation-in-part of international application number PCT/EP2010/050426, filed Jan. 14, 2010, which claims the benefit of application numbers GB 0900514.1, filed Jan. 14, 2009, NL 1036427, filed Jan. 15, 2009, GB 0909894.8, filed Jun. 9, 2009, GB 0910048.8, filed Jun. 11, 2009, GB 0919448.1, filed Nov. 5, 2009, and GB 0920456.1, filed Nov. 24, 2009, the disclosures of which are hereby incorporated by reference in their entireties. This application also claims the benefit of U.S. provisional application No. 61/363,852, filed Jul. 13, 2010, the disclosure of which is hereby incorporated by reference in its entirety.

### FIELD OF THE INVENTION

The invention relates to life saving medicaments for critically ill patients and novel methods of treating a critically ill patient. The invention relates to methods and preparations to increase the survivability of critically ill patients and to reduce or prevent the risk of mortality of the critically ill, mortality due to multiple organ failure and muscle weakness. The invention further relates to methods and preparations for protecting the critically ill, who are subjected to parenteral nutrition, against multiple organ failure or muscle weakness caused by parenteral nutrient delivery, particularly by unbalanced parenteral nutrition or a parenteral nutrient delivery that creates (relative) nutrient overload.

### BACKGROUND OF THE INVENTION

Thanks to major advances in intensive care medicine, critically ill patients nowadays often survive acute conditions that were previously lethal. Despite much effort, however, the mortality of patients who survive this initial phase and enter a chronic phase of critical illness remains high worldwide. In these patients, mortality is often due to non-resolving multiple organ failure and muscle weakness. Treatments that have been introduced to improve the weakness, such as hyperalimentation, growth hormone, or androgens, failed because these interventions unexpectedly increased the risk of organ failure and death.

Critically ill or injured patients, particularly the prolonged critically ill, have nutritional needs that are often not, or insufficiently, met by enteral formulas. Severe injury or trauma, including surgery, is associated with loss of the body's nutrient stores due both to the injury itself and the resulting catabolic response. For optimal recovery, critically ill patients need proper nutritional intake. Lack of it can result in malnutrition-associated complications, including prolonged negative nitrogen balance, depletion of somatic and visceral protein levels, immune incompetence, increased risk of infection, and other complications associated with morbidity and mortality. Hormonally mediated hypermetabolism, catabolism, elevated basal metabolic rate and nitrogen excretion, altered fluid and electrolyte balance, synthesis of acute phase proteins, inflammation, and immunosuppression are often observed after severe injury, major surgery, or critical illness. Both anabolic and catabolic processes are accelerated following severe trauma, although catabolism predominates. This response allows muscle breakdown to occur in order to provide amino acids for synthesis of proteins involved in immunological response and tissue repair. Disuse atrophy contributes to the muscle wasting and negative nitrogen balance frequently observed in the trauma patient and the critically ill patient.

A primary objective of nutritional support for the injured or ill person is to replace or maintain the body's normal level of nutrients by providing adequate energy substrates, protein, and other nutrients essential for tissue repair and recovery. The nutritional support of trauma and surgery patients has been extensively investigated in the prior art. Recently, it has been suggested and documented that the nutritional support to trauma and surgery patients may also have detrimental effects (Vanhorebeek et al. (2005) Lancet 365, 53-59).

U.S. Pat. No. 5,576,350 relates to a method for the prophylaxis of shock in a patient induced by endotoxin or bacteremia is disclosed. The method involves administering a therapeutically effective amount of a chemical composition dissolved in a pharmaceutically compatible solvent, such as a phosphate buffered saline, to the patient. The preferred chemical composition is spermidine, which binds to bacterial lipopolysaccharides.

Rasanen et al. (US 20040180968) and Hyvonen et al. (2006) Am. J. Pathol. 168, 115-122, and Doctoral Dissertation of December 2007, 2007, University of Kuopio, Finland disclose the use of spermine in the prevention of pancreatitis and the use of polyamines, modified at one or more of their NH₂ or NH groups in the treatment of pancreatitis.

There is a need in the art to provide critically ill patients with appropriate treatments and adequate nutrition.

### SUMMARY OF THE INVENTION

The present invention demonstrates the beneficial effects of autophagy inducing agents such as polyamines to improve the condition of critically ill patients who suffer from multiple organ dysfunction. These polyamines allow to ameliorate the condition of critically ill patients.

The above objective is accomplished by polyamine compounds, pharmaceutical compositions, methods and uses of a polyamine compound to manufacture a medicament according to the present invention or to treat critically ill patients who suffer from multiple organ dysfunction, in particular such enhanced or caused by administered nutrient overload for instance by force feeding, tubefeeding for instance by receiving enteral or parenteral nutrition delivery.

One aspect of the present invention relates to the use of autophagy inducing agents such as a polyamine or a salt, solvate, or derivative thereof for the treatment or prevention of a life threatening condition in a critically ill patient with a non-infectuous disorder.

In particular embodiments of uses according to the present invention, the polyamine is a metabolisable polyamine, more particularly, the polyamine is a substrate for the enzyme Spermine/Spermidine Acetyltranferase (SSAT), more particularly the polyamine is not modified (particularly not methylated at) one or more of the NH₂ or NH groups.

In particular embodiments of uses according to the present invention, the polyamine is selected from the group consisting of putrescine (1,4-diamino-butane), 1,3-diamino-propane, 1,7-diamino-heptane, 1,8-diamino-octane, spermine, spermidine, cholesteryl spermine, spermidine trihydrochloride, spermidine phosphate hexahydrate, spermidine phosphate hexahydrate, L-arginyl-3,4-spermidine and 1,4-butanediamine N-(3-aminopropyl)-monohydrochloride, more particularly spermine or spermidine or the polyamine is a methylated spermidine or methylated spermine analogue for instance of the group of N1-(4-Amino-butyl)-butane-1,3-diamine, N1-[4-(3-Amino-propylamino)-butyl]-butane-1,3-diamine, N1-(3-Amino-2-methyl-propyl)-butane-1,4-diamine, N1-(3-Amino-propyl)-pentane-1,4-diamine, N1-(3-Amino-butyl)-pentane-1,4-diamine, N1-(4-Amino-butyl)-3-methyl-butane-1,3-diamine, N1-(3-Amino-2,2-dimethyl-propyl)-butane-1,4-diamine, N3-(4-Amino-butyl)-3-methyl-butane-1,3-diamine, N1-(3-Amino-propyl)-4-methyl-pentane-1,4-diamine and N1-[4-(3-Amino-butylamino)-butyl]-butane-1,3-diamine, which are not methylated at one or more of the NH2 or NH groups or the polyamine is Sulfuric acid mono-(4-{3-[3-(4-amino-butylamino)-propylamino]-7-hydroxy-10,13-dimethyl-hexadecahydro-cyclopenta[a]phenanthren-17-yl}-1-isopropyl-pentyl) ester.

A particular embodiments of uses according to the present invention the polyamine is a squalamine for instance a squalamine spermindine analogue selected of the group consisting of Cholestane-7,24-diol, 3-[[3-[(4-aminobutyl)amino]propyl]amino]-, 24-(hydrogen sulfate), (3.beta., 5.alpha., 7.alpha., 24R)-, (2S)-2-hydroxypropanoate (1:1), Cholestane-7,24-diol, 3-[[3-[(4-aminobutyl)amino]propyl]amino]-, 24-(hydrogen sulfate), (3.beta., 5.alpha., 7.alpha., 24R)- and Sulfuric acid mono-(4-{3-[3-(4-amino-butylamino)-propylamino]-7-hydroxy-10,13-dimethyl-hexadecahydro-cyclopenta[a]phenanthren-17-yl}-1-isopropyl-pentyl) ester.

Yet another particular embodiments of uses according to the present invention the polyamine is a spermindine analogue selected of the group consisting of

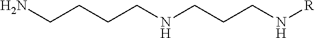

wherein R is an optionally substituted C1-12 alkyl, C3-20 heterocyclyl and C5-20 aryl groups, an optionally substituted C5-20 aryl group,
wherein the optional substituent are independently selected from the group consisting of C1-12 alkyl, C3-12 cycloalkyl, C3-20 heterocyclyl, C5-20 aryl, halo, hydroxyl, —OR1 wherein R1 is a C1-7 alkyl group or C3-20 heterocyclyl group or C5-10 aryl group, alkoxy, —CH(OR1)(OR2) wherein R1 is as defined above and R2 is independently a C1-7 alkyl group or C3-20 heterocyclyl group or C5-10 aryl group or R1 and R2 together with the two oxygen atoms to which they are attached form a heterocyclic ring having from 4 to 8 ring atoms, —CH(OH)(OR1) wherein R1 is as defined above, ketal, hemiketal, oxo, thione, imino, formyl, acyl, carboxy, thiocarboxy, thiolocarboxy, —C(═NH)OH, —C(═NOH)OH, —C(═O)OR1 wherein R1 is as defined above, acyloxy, oxycarboyloxy, amino, amido, thioamido, acylamido, aminocarbonyloxy, ureido, guanidine, tetrazolyl, amindino, nitro, nitroso, azido, cyano, isocyano, cyanato, isocyanato, thiocyano, isothiocyano, sulfhydryl, thioether, disulfide, sulfine, sulfone, —S(═O)OH, —SO2H, —S(═O)2OH, —SO3H, sulfinate, sulfonate, sulfinyloxy, sulfonyloxy, sulfate, sulfamyl, sulfonamide, sulfamino, sulfonamino, sulfinamino, phosphino, phosphor, phosphinyl, phosphono, —P(═O)(OR17)2 wherein R17 is —H or C1-7 alkyl group or C3-20 heterocyclyl group or C5-20 aryl group, phosphonooxy, —PO(═O)(OR17)2 wherein R17 is as defined above, —OP(OH)2, phosphate, phosphoramidite, and phosphoramidate; and wherein heteroatoms of the heterocyclyl groups and the optional heteroatoms of the alkylene groups are independently selected from the group consisting of N, S, and O.

In particular embodiments of uses according to the present invention, the life threatening condition is selected from the group consisting of lactic acidosis, muscle weakening, hyperglycemia, multiple organ failure and failed or disturbed homeostasis.

In particular embodiments of uses according to the present invention, the critically ill patient is a patient receiving enteral or parenteral nutrition, wherein the polyamine is e.g. administered together with an enteral or parenteral nutritient composition. Such polyamine is preferably a molecule with a spermidine structure or a functional group selected from N1-(3-Methylamino-propyl)-butane-1,4-diamine, N1-(4-Amino-butyl)-N3-methyl-butane-1,3-diamine, N1-[4-(3-Amino-propylamino)-butyl]-N3-methyl-butane-1,3-diamine N1-(2-Methyl-3-methylamino-propyl)-butane-1,4-diamine, N1-(3-Amino-propyl)-pentane-1,4-diamine, N1-(3-Methylamino-butyl)pentane-1,4-diamine, N1-(4-Amino-butyl)-3, N3-dimethyl-butane-1,3-diamine, N1-(2,2-Dimethyl-3-methylamino-propyl)-butane-1,4-diamine, N3-(4-Amino-butyl)-3, N1-dimethyl-butane-1,3-diamine, N3-Methyl-N1-[4-(3-methylamino-propylamino)-butyl]-butane-1,3-diamine, N1-(3-Amino-propyl)-4-methyl-pentane-1,4-diamine, N1-[4-(3-Amino-butylamino)-butyl]-N3-methyl-butane-1,3-diamine or N1-[4-(3-Methylamino-butylamino)-butyl]-butane-1,3-diamine. One aspect of present invention is a compound selected of this group for use in a treatment for treating or preventing of a life threatening condition in a critically ill patient with a non-infectious disorder, in particular such disorder caused or enhanced by caused or enhanced by unbalanced parenteral nutrition or a parenteral nutrient delivery that creates (relative) nutrient overload.

In particular embodiments of uses according to the present invention, the disorder of the critically ill patient is selected from the group consisting of severe or multiple trauma, high risk or extensive surgery, cerebral trauma or bleeding, respiratory insufficiency, abdominal peritonitis, acute kidney injury, acute liver injury, severe burns and critical illness polyneuropathy and in particular such caused or enhanced by unbalanced parenteral nutrition or a parenteral nutrient delivery that creates (relative) nutrient overload.

Another aspect of the present invention relates to the use of an autophagy inducing agents such as a polyamine, or a salt, solvate, or derivative thereof, and preferably such polyamine with a spermidine or spermine structural group, for manufacture of a medicament for the treatment or prevention of a life threatening condition in a critically ill patient with a non-infectious disorder.

In particular embodiments of uses according to the present invention, the critically ill patient is a patient receiving enteral or parenteral nutrition, wherein the polyamine, preferably such polyamine with a spermidine or spermine structural group, is administered together with an enteral or parenteral nutrient composition.

Another aspect of the invention relates to nutrient solution suitable for parenteral administration, said nutrient solution comprising a saccharide, characterised in that said solution further comprises a polyamine or a salt, solvate, or derivative thereof, preferably such polyamine with a spermidine or spermine structural group. In particular embodiments, the solution is suitable for intravenous administration.

Another aspect of the present invention relates to the use of a saccharide and a polyamine or a salt, solvate, or derivative thereof as a medicament.

Another aspect of the present invention relates to the use of a saccharide and a polyamine or a salt, solvate, or derivative thereof, preferably such polyamine with a spermidine or spermine structural group, for the treatment or prevention of a life threatening condition in a critically ill patient with a non-infectious disorder.

Another aspect of the present invention relates to the use of a saccharide and a polyamine or a salt, solvate, or derivative, preferably such polyamine with a spermidine or spermine structural group, for the manufacture of a medicament for the treatment or prevention of a life threatening condition in a critically ill patient with a non infectious disorder.

In particular embodiments the medicament is a solution suitable for intravenous administration.

Another aspect of the present invention relates to a method of treating a life threatening condition in a critically ill patient with a non infectious disorder comprising the step of administering to said patient zn autophagy inducing agents such as a polyamine, or a salt, solvate, or derivative thereof.

Another aspect of the present invention relates to a pharmaceutical composition comprising a nutrient solution comprising autophagy inducing agents such as a polyamine or a salt, solvate, or derivative thereof, preferably such polyamine with a spermidine or spermine structural group, as described above for the treatment or prevention of a life threatening condition in a critically ill patient with a non-infectious disorder, administering said a polyamine or a salt, solvate, or derivative thereof in one or more doses between 50 μg and 10 gram per day, depending on the body weight, e.g. between 10 μg/kg body weight/day to about 100 mg/kg/day.

It has been surprisingly found that multiple organ dysfunction in living organisms can be treated by polyamine compounds of the groups consisting of putrescine, spermine, spermidine, cholesteryl spermine, spermidine trihydrochloride, spermidine phosphate hexahydrate, spermidine phosphate hexahydrate and 1,4-butanediamine N-(3-aminopropyl)-monohydrochloride or derivatives thereof or combinations thereof. Such multiple organ dysfunction, common in the critical care setting, can be caused or aggravated by unbalanced parenteral nutrient delivery or a parenterally delivered relative or absolute nutrient overload.

It was found that a treatment by polyamine compounds of the groups consisting of putrescine, spermine, spermidine, cholesteryl spermine, spermidine trihydrochloride, spermidine phosphate hexahydrate, spermidine phosphate hexahydrate, 1,4-butanediamine N-(3-aminopropyl)-monohydrochloride and squalamine or derivatives thereof or combinations thereof can increase the survivability of critically ill patients and can reduce or prevent the risk of mortality of the critically ill which is due to multiple organ failure that does not resolve or heal and to muscle weakness. Such treatment or preparation protects the critically ill, who are subjected to parenteral nutrition, against mortality or morbidity of multiple organ failure or of muscle weakness, in particular when such multiple organ failure or of muscle weakness is caused or aggravated by parenteral nutrient delivery, which may be unbalanced in this context, or parenterally delivered relative or absolute nutrient overload.

The compounds used in the present invention ameliorate the condition of critically ill patients, provide a treatment to treat or to prevent multiple organ dysfunction in the critically ill, provide a treatment to prevent mitochondrial dysfunction induced by inadequate or unbalanced parenteral nutrition to the critically ill and increase the survivability or reduce mortality in such critically ill patients.

The present invention demonstrates that multiple organ dysfunction can be treated in cells, tissues and living organisms by polyamine compounds more in particular polyamines wherein the NH₂ or NH group are not modified, such that they remain a substrate for acetylating enzymes. Examples hereof are putrescine, spermine, spermidine, cholesteryl spermine, spermidine trihydrochloride, spermidine phosphate hexahydrate, spermidine phosphate hexahydrate and 1,4-butanediamine N-(3-aminopropyl)-monohydrochloride or derivatives thereof or combinations thereof.

A particular embodiment relates to a polyamine compound of the group consisting of putrescine (1,4-diamino-butane), 1,3-diamino-propane, 1,7-diamino-heptane, 1,8-diamino-octane, spermine, spermidine, or a derivative thereof or a pharmaceutically acceptable salt, solvate or isomer thereof, such as cholesteryl spermine, spermidine trihydrochloride, spermidine phosphate hexahydrate, spermidine phosphate hexahydrate, and 1,4-butanediamine N-(3-aminopropyl)-monohydrochloride or combinations thereof, for use in a treatment of treating or preventing multiple organ dysfunction in a critically ill patient.

In a preferred embodiment, the polyamine compound of present invention is spermidine or a pharmaceutically acceptable salt, solvate or isomer thereof, or combinations thereof.

It is an advantage of present invention that the polyamine compound can be used in a treatment of multiple organ dysfunction wherein the polyamine compound is administered parenterally or enterally to the critically ill patient.

Another aspect of the invention relates to a pharmaceutical composition comprising a pharmacologically acceptable amount of a polyamine compound of the group consisting of putrescine (1,4-diamino-butane), 1,3-diamino-propane, 1,7-diamino-heptane, 1,8-diamino-octane, spermine, spermidine, or a derivative thereof or a pharmaceutically acceptable salt, solvate or isomer thereof, such as cholesteryl spermine, spermidine trihydrochloride, spermidine phosphate hexahydrate, spermidine phosphate hexahydrate, and 1,4-butanediamine N-(3-aminopropyl)-monohydrochloride or combinations thereof, for use in a treatment of treating or preventing multiple organ dysfunction in a critically ill patient.

In a preferred embodiment, the pharmaceutical composition of present invention comprises a polyamine compound, which is a pharmacologically acceptable amount of spermidine or a pharmaceutically acceptable salt, solvate or isomer thereof, or combinations thereof.

It is an advantage of the pharmaceutical composition that the pharmaceutical composition can be provided as an aqueous liquid composition. Moreover, it is advantageous that the pharmaceutical composition can be administered parenterally or enterally to the critically ill patient. In a preferred embodiment, the critically ill patient further receives total parenteral nutrition.

It is an advantage of the pharmaceutical composition that the pharmaceutical composition can be provided to normalize the plasma spermidine level in the critically ill patient.

In particular embodiments dried polyamine comprising compositions are reconstituted with water to the pharmaceutical composition of present invention.

A further aspect of the invention relates to a method to treat or to prevent multiple organ dysfunction in a critically ill patient by administering to the critically ill patient a pharmaceutical composition comprising a pharmacologically acceptable amount of a polyamine compound of the group consisting of putrescine (1,4-diamino-butane), 1,3-diamino-propane, 1,7-diamino-heptane, 1,8-diamino-octane, spermine, spermidine, or a derivative thereof or a pharmaceutically acceptable salt, solvate or isomer thereof such as cholesteryl spermine, spermidine trihydrochloride, spermidine phosphate hexahydrate, spermidine phosphate hexahydrate, and 1,4-butanediamine N-(3-aminopropyl)-monohydrochloride, or combinations thereof, for use in a treatment of treating or preventing multiple organ dysfunction in a critically ill patient.

In a preferred embodiment, the method to treat or to prevent multiple organ dysfunction in a critically ill patient comprises the step of administering to the critically ill patient a pharmaceutical composition comprising a polyamine compound which is a pharmacologically acceptable amount of spermidine or a pharmaceutically acceptable salt, solvate or isomer thereof, or combinations thereof.

It is an advantage that the method of present invention can normalize the plasma spermidine level in the critically ill patient.

A further aspect of the invention relates to the use of a polyamine compound of the group consisting of putrescine (1,4-diamino-butane), 1,3-diamino-propane, 1,7-diamino-heptane, 1,8-diamino-octane, spermine, spermidine, or a derivative thereof or a pharmaceutically acceptable salt, solvate or isomer thereof, such as cholesteryl spermine, spermidine trihydrochloride, spermidine phosphate hexahydrate, spermidine phosphate hexahydrate, and 1,4-butanediamine N-(3-aminopropyl)-monohydrochloride or combinations thereof, to manufacture a medicament to treat or prevent multiple organ dysfunction in a critically ill patient.

A further aspect of the invention relates to the use of a polyamine compound of the group consisting of N1-(4-Amino-butyl)-butane-1,3-diamine, N1-[4-(3-Amino-propylamino)-butyl]-butane-1,3-diamine, N1-(3-Amino-2-methyl-propyl)-butane-1,4-diamine, N1-(3-Amino-propyl)-pentane-1,4-diamine, N1-(3-Amino-butyl)-pentane-1,4-diamine, N1-(4-Amino-butyl)-3-methyl-butane-1,3-diamine, N1-(3-Amino-2,2-dimethyl-propyl)-butane-1,4-diamine, N3-(4-Amino-butyl)-3-methyl-butane-1,3-diamine, N1-(3-Amino-propyl)-4-methyl-pentane-1,4-diamine, N1-[4-(3-Amino-butylamino)-butyl]-butane-1,3-diamine Cholestane-7,24-diol, 3-[[3-[(4-aminobutyl)amino]propyl]amino]-, 24-(hydrogen sulfate), (3.beta., 5.alpha., 7.alpha., 24-, (2S)-2-hydroxypropanoate (1:1), Cholestane-7,24-diol, 3-[[3-[(4-aminobutyl)amino]propyl]amino]-, 24-(hydrogen sulfate), (3.beta., 5.alpha., 7.alpha., 24R)- and Sulfuric acid mono-(4-{3-[3-(4-amino-butylamino)-propylamino]-7-hydroxy-10,13-dimethyl-hexadecahydro-cyclopenta[a]phenanthren-17-yl}-1-isopropyl-pentyl) ester, or a derivative thereof or a pharmaceutically acceptable salt, solvate or isomer thereof eventually in a medicament with a carrier for use in a treatment to treat or to prevent multiple organ dysfunction in a critically ill patient.

In a preferred embodiment, the use of the polyamine compound of present invention is the use of spermidine or a pharmaceutically acceptable salt, solvate or isomer thereof, or combinations thereof.

It is an advantage of present invention that the polyamine compound is used to manufacture a medicament to treat or prevent multiple organ dysfunction wherein the polyamine compound is administered parenterally or enterally to the critically ill patient. It is an advantage of embodiments of present invention that polyamine compounds and pharmaceutical compositions administered to critically ill patients suffering from multiple organ failure have a decreased length of time spent on ventilator.

In preferred embodiment, the method to treat or to prevent multiple organ dysfunction in a critically ill patient comprises the step of administering to the critically ill patient a pharmaceutical composition comprises a polyamine compound which is a pharmacologically acceptable amount of spermidine or a pharmaceutically acceptable salt, solvate or isomer thereof, or combinations thereof.

It is an advantage that the method of present invention can normalize the plasma spermidine level in the critically ill patient.

A further aspect of the present invention relates to the use of a polyamine compound of the group consisting of putrescine, (1,4-diamino-butane), 1,3-diamino-propane, 1,7-diamino-heptane, 1,8-diamino-octane, spermine, spermidine, cholesteryl spermine, spermidine trihydrochloride, spermidine phosphate hexahydrate, spermidine phosphate hexahydrate, and 1,4-butanediamine N-(3-aminopropyl)-monohydrochloride or a derivative thereof or a pharmaceutically acceptable salt, solvate or isomer thereof, or combinations thereof, to prevent mitochondrial dysfunction induced by inadequate or unbalanced parenteral nutrition delivered to a critically ill patients.

A further aspect of the present invention relates to the use of a polyamine compound of the group consisting of N1-(4-Amino-butyl)-butane-1,3-diamine, N1-[4-(3-Amino-propylamino)-butyl]butane-1,3-diamine, N1-(3-Amino-2-methyl-propyl)-butane-1,4-diamine, N1-(3-Amino-propyl)-pentane-1,4-diamine, N1-(3-Amino-butyl)-pentane-1,4-diamine, N1-(4-Amino-butyl)-3-methyl-butane-1,3-diamine, N1-(3-Amino-2,2-dimethyl-propyl)-butane-1,4-diamine, N3-(4-Amino-butyl)-3-methyl-butane-1,3-diamine, N1-(3-Amino-propyl)-4-methyl-pentane-1,4-diamine, N1-[4-(3-Amino-butylamino)-butyl]-butane-1,3-diamine Cholestane-7,24-diol, 3-[[3-[(4-aminobutyl)amino]propyl]amino]-, 24-(hydrogen sulfate), (3.beta., 5.alpha., 7.alpha., 24-, (2S)-2-hydroxypropanoate (1:1), Cholestane-7,24-diol, 3-[[3-[(4-aminobutyl)amino]propyl]amino]-, 24-(hydrogen sulfate), (3.beta., 5.alpha., 7.alpha., 24R)- and Sulfuric acid mono-(4-{3-[3-(4-amino-butylamino)-propylamino]-7-hydroxy-10,13-dimethyl-hexadecahydro-cyclopenta[a]phenanthren-17-yl}-1-isopropyl-pentyl) ester, or a derivative thereof or a pharmaceutically acceptable salt, solvate or isomer thereof or a combination to prevent mitochondrial dysfunction induced by inadequate or unbalanced parenteral nutrition delivered to a critically ill patients.

A further aspect of the present invention relates to the use of a squalamine polyamine compound or a derivative thereof or a pharmaceutically acceptable salt, solvate or isomer thereof or a combination to prevent mitochondrial dysfunction induced by inadequate or unbalanced parenteral nutrition delivered to critically ill patients

Another aspect of the present invention relates to a method to treat or to prevent mitochondrial dysfunction in a critically ill patient comprising the step of administering to the critically ill patient a pharmaceutical composition comprising a polyamine compound which is a pharmacologically acceptable amount of spermidine or a pharmaceutically acceptable salt, solvate or isomer thereof, or combinations thereof. It is an advantage that such method can normalize the plasma spermidine level in the critically ill patient.

The invention relates further to a pharmaceutical composition comprising a pharmacologically effective amount of a polyamine as described herein as a pharmaceutically suitable carrier. In aqueous liquid composition the polyamine concentration can range from 0.05% to about 4%, or from about 0.5% to about 2% or from about 1.0% to about 1.5% of said aqueous liquid composition.

Such a pharmaceutical composition can further comprise a blood glucose regulator and or comprising nutrients.

The methods and compositions of the present invention are for normalising the plasma spermidine level in said critically ill patient, or to augment the plasma spermidine level in said critically ill patient to a level that is 1 to 2.5 times, 4 or even 5 times the plasma spermidine level of a healthy person with a similar body weight as said critically ill patient, for example to augment the plasma spermidine level in said critically ill patient to a level that is about twice the plasma spermidine level of a healthy person with a similar body weight as said critically ill patient or for example to augment the plasma spermidine level in said critically ill patient to a level that is restoring the plasma spermidine level to that of a healthy person with a similar body weight as said critically ill patient.

In particular embodiments the treatment is a treatment to augment the plasma spermidine level in said critically ill patient to a level in the range of 50 to 6000 nmol/l plasma, to augment the plasma spermidine level in said critically ill patient to a level in the weight range of 100 to 6000 nmol/l plasma, to augment the plasma spermidine level in said critically ill patient by administering daily said polyamine compound in the weight range of 0.05-1, 1-200, 5-150, 10-120 mg, or 40-80 mg per kg body weight.

Typically between 50 μg to 10 g of a polyamine compound, preferably a spermidine compound, per daily serving in one or more portion is administered to a human critically ill patient.

Polyamines as described in the present invention can be administered parenterally or enterally to a critically ill patient, or by a bolus injection or by an intravenous bolus injection to said critically ill patient.

Polyamines as described in the present invention are suitable for inter alia;
treatment of multiple organ dysfunction in a critically ill patient with failed or disturbed homeostasis receiving parenteral nutrition.
protection a critically ill patient against multiple organ dysfunction by inducing adipocytes dedifferentiation. treatment of the development of lactic acidosis (lowering of blood pH and an increase in lactate),
treatment of preventing parenteral nutrition induced development of lactic acidosis,
treatment of treating or preventing muscle weakness in a critically ill patient.
decreasing or preventing parenteral nutrition aggravated morbidity or mortality in the critically ill patient,
preventing body system collapse,
treatment of preventing morbidity or mortality in a critically ill patient,
treatment of preventing development of lactic acidosis in a critically ill patient.

The compositions, methods and uses of the present invention provide several advantages such as:
the condition of critically ill animals or critically ill humans is improved.
excessive catabolism in a critically ill patient is prevented or treated.
morbidity or mortality due to excessive catabolism, e.g. in a critically ill patient, is reduced.
the polyamine compound can be administered intravenously.
multiple organ dysfunction syndrome in a critically ill patient can be reversed, treated or cured.
the polyamine compound can be provided in an economically viable way.
the polyamine compound can be administered in a pharmacologically acceptable way.

Particular and preferred aspects of the invention are set out in the accompanying independent and dependent claims. Features from the dependent claims may be combined with features of the independent claims and with features of other dependent claims as appropriate and not merely as explicitly set out in the claims.

The above and other characteristics, features and advantages of the present invention will become apparent from the following detailed description, taken in conjunction with the accompanying figures, which illustrate, by way of example, the principles of the invention. This description is given for the sake of example only, without limiting the scope of the invention. The reference figures quoted below refer to the attached figures.

### BRIEF DESCRIPTION OF THE DRAWINGS

FIG. 1: Flow chart of the treatment of critically ill rabbits according to embodiments of present invention.

FIG. 2: Adjustment of glucose and insulin infusions in burn-injured rabbits of group 1 and group 2 according to embodiments of present invention.

FIG. 3: Flow chart of the treatment of critically ill rabbits according to embodiments of present invention.

FIG. 4: Adjustment glucose and insulin infusions in burn-injured rabbits of groups 1-6 according to an embodiment of present invention.

FIG. 5: Graphs showing glucose uptake and transporters in healthy versus critically ill patients. The data represent (A) gene expression levels (mRNA A.U.) and (B) protein translation levels (protein A.U.) of GLUT1, GLUT3, GLUT4, as well as (C) glucose levels (μmol/g tissue) of subcutaneous (Subc. A.T.) and omental (Omental A.T.) adipose tissue biopsies, and (D) serum glucose levels (mg/dl) of 61 prolonged critical ill patients (taken minutes after death) and of 20 non-critically ill patients (taken during abdominal surgery).

FIG. 6: Graphs showing lipogenesis in healthy versus critically ill patients. The data represent: (A) ACC protein translation levels (A.U.), (B) FAS activity (% cpm of control), and (C) SCD gene expression levels (mRNA A.U.) of subcutaneous (Subc. A.T.) and omental (Omental A.T.) adipose tissue biopsies, as well as (D) serum insulin levels (mlU/I) and (E) serum triglyceride levels (mg/dl) of 61 prolonged critical ill patients (taken minutes after death) and of 20 non-critically ill patients (taken during abdominal surgery).

FIG. 7: Graphs showing adipose tissue morphology in healthy versus critically ill patients. The data represent: (A) median cell area (μm2) and (B) perilipin gene expression levels (mRNA A.U.) of subcutaneous (Subc. A.T.) and omental (Omental A.T.) adipose tissue biopsies of 61 prolonged critical ill patients (taken minutes after death) and of 20 non-critically ill patients (taken during abdominal surgery).

FIG. 8: Picture illustrating Cd68 (macrophage) coloring in healthy versus critically ill patients.

FIG. 9: (A) Cell line chart showing cell mean in critically ill rabbits (sperm d7.2) [right)] and healthy control rabbits (sperm baseline.2) [left] at day 7, data representing 8 animals per group. (B) Box plot showing spermidine levels in plasma of critically ill rabbits (sperm d7.2) [right] and healthy control rabbits (sperm baseline.2) [left] at day 7, data representing 8 animals per group.

FIG. 10: (A) Cell line chart showing cell mean in critically ill rabbits (sperm d7.2) and healthy control rabbits (sperm baseline.2) at day 7, data representing 7 animals per group. (B) Box plot showing spermidine levels in plasma of critically ill rabbits (sperm d7.2) and healthy control rabbits (sperm baseline.2) at day 7, data representing 7 animals per group; (C). Box plot showing spermidine levels (ng/ml plasma) in critically ill rabbits (from day −1 to day 7 after injury upon application of various doses of spermidine) (D) detail of (C).

FIGS. 11 and 12 show a decrease in mortality of parenterally fed hyperglycemic critically ill animals receiving spermidine.

FIGS. 13A and 13B show a decrease in mortality of parenterally fed hyperglycemic critically ill animals receiving spermidine.

FIGS. 14 and 15 show a different time course of blood pH and lactate levels in surviving and non-surviving animals, with early differences between both groups (considerable time before the first animals died).

FIG. 16 shows that spermidine administration prevents an increase of creatinine, a marker of renal failure.

FIG. 17 shows that spermidine administration decreases the levels of ureum, a marker of renal failure and of muscle wasting.

FIG. 18 shows that spermidine administration decreases the levels of AST (ASpartate Transaminase), a marker of liver failure.

FIG. 19 shows the effect of feeding versus fasting on the markers of mitochondrial activity in liver, in critically ill animals, supporting the indication for spermidine as a mitochondrial protective agent with feeding.

FIG. 20 shows that spermidine leads to hypoacetylation of histone H3 and strongly induces autophagy. (A) Relative acetylation (normalized to respective controls, dashed line) of indicated histone H3 lysine residues determined by quantification of immunoblot analysis using site specific antibodies. Data represent means of two independent analyses. For calculation details and representative blots (Balasundaram et al. Proc. Natl. Acad. Sci. USA 88, 5872-5876). (B) Relative acetylation of indicated histone H3 lysine residues of Δspe1 cells (open bars) compared to wild type cells (closed bars) chronologically aged to day 3 (Lys18 and Lys56) or day 12 (Lys9+14). Data represent means of two independent analyses. (C) Relative inhibition of histone acetyltransferase activity (HAT-activity) by spermidine determined by an in vitro HAT-activity assay of yeast nuclear extracts of wild type cells. Data represent means±SEM of three independent experiments. *p=0.024. (D) Chronological aging of wild type and Δiki3Δsas3 with (open symbols) or without (closed symbols) addition of 4 mM spermidine. Data represent means±SEM (n=4). (E) Relative acetylation of histone H3 lysine 9+14 residues determined by quantification of immunoblot analysis performed at day 20 of the experiment shown in (E). Data represent means±SEM (n=3). *p<0.05, ***p<0.001.

FIG. 21 shows that application of spermidine extends life span of yeast and inhibits oxidative stress in aging mice. Survival determined by clonogenicity during chronological aging of wild type yeast (BY4741) with (∘) and without (▪) addition of 4 mM spermidine at day 1. Data represent means±SEM (n=5).

FIG. 22 shows that spermidine treatment of yeast results in strong resistance against heat shock and peroxide treatment. Survival of pre-aged wild type cells stressed for 4 h with hydrogen peroxide (3 mM H₂O₂) or heat shock (42° C.) compared to unstressed cells. Cells were chronologically aged until day 24 with or without addition of 4 mM spermidine. Data represent means±SEM (n=4). *p<0.05 and ***p<0.001. B) Free thiol group concentration (indicative of oxidative stress level) in blood serum of aging mice with or without (control) supplementation of drinking water with 0.3 and 3 mM spermidine for 200 days. Data represent means±SEM (n=3). **p<0.01. (C) Intracellular spermidine of mouse liver cells, obtained from the same mice used for RSH measurements presented in (B). Data represent means±SEM (n=3).

FIG. 23 shows that histone H3 acetylation is regulated by intracellular polyamines in part mediated through Iki3p and Sas3p. (A) Immunoblot of whole cell extracts of wild type cells chronologically aged to designated time points with (+) or without (−) spermidine application. Blots were probed with antibodies against total histone H3 or H3 acetylation sites at the indicated lysine residues. (B) Relative acetylation of histone H3 lysine 9+14 of Δspe1 cells compared to wild type cells chronologically aged to day 5 with (open bars) or without (closed bars) adjustment of pHₑₓ to 6. Data represent means±SEM of three independent experiments. **p<0.01. (C) Quantification (FACS analysis) of phosphatidylserine externalization and loss of membrane integrity using AnnexinV/PI co-staining performed at day 20 of the chronological aging experiment shown in (FIG. 20D). For each staining 30,000 cells were evaluated. ***p<0.001. (D) Immunoblot of whole cell extracts of wild type and Δiki3Δsas3 cells with (+) or without (−) spermidine application obtained at day 20 of the aging experiment shown in (FIG. 20D). Blots were probed with antibodies against total histone H3 or H3 acetylation sites at lysine 9+14 (Lys9+14).

FIG. 24 is a graphic display showing that polyamine depletion shortens yeast chronological life span evoking markers of oxidative stress and necrosis. (A) Intracellular spermidine of five-day-old Δspe1 cells compared to wild type. Data represent means±SEM (n=3). ***p<0.001. (B) Chronological aging of wild type (▪) and polyamine depleted Δspe1 (Δ) yeast cells. Data represent mean±SEM (n=6). Cells were tested for cell death markers at day 3 (C-E). (C) Fluorescence microscopy of DHE stained wild type and Δspe1 cells indicating ROS accumulation. Scale bars represent 10 μm. (D) Quantification (fluorescence reader) of ROS accumulation using DHE staining of wild type and Δspe1 cells. Data represent means±SEM (n=4). ***p<0.001. (E) Quantification (FACS analysis) of phosphatidylserine externalization and loss of membrane integrity using Annexin V/PI costaining and of DNA-fragmentation using TUNEL staining of chronologically aging wild type and Δspe1 cells at day 3. Data represent means±SEM (n=3). **p<0.01.

FIG. 25 shows life span extension upon external alkalinization strictly depends on endogenous polyamines. (A) Chronological aging of wild type (closed symbols) and Δspe1 (open symbols) with (▴) and without (▪) adjustment of extracellular pH to 6. Data represent means±SEM (n=3). (B) Intracellular pH determined by staining with the pH-dependent fluorescent dye SNARF-4F of wild type and Δspe1 cells with and without adjustment of extracellular pH to 6 during chronological aging. Data represent means±SEM (n=3). *p<0.05, **p<0.01, and ***p<0.001.

FIG. 26 shows that spermidine application suppresses necrotic cell death. (A)

Fluorescence microscopy of DHE staining and Annexin V/PI costaining of wild type cells at day 18 of the chronological aging experiment. Scale bars represent 10 μm. (B and C) Quantification of DHE staining (B) and Annexin V/PI costaining (C) by FACS analysis performed at indicated time points of the chronological aging experiment. Data represent means±SEM (n=3). ***p<0.001. (D) Fluorescence microscopy of chronologically aged wild type cells (day 3 and 14) expressing an EGFP-tagged version of the yeast HMGB1 homolog (Nhp6A-EGFP) with or without (control) addition of 4 mM spermidine. Scale bars represent 5 μm

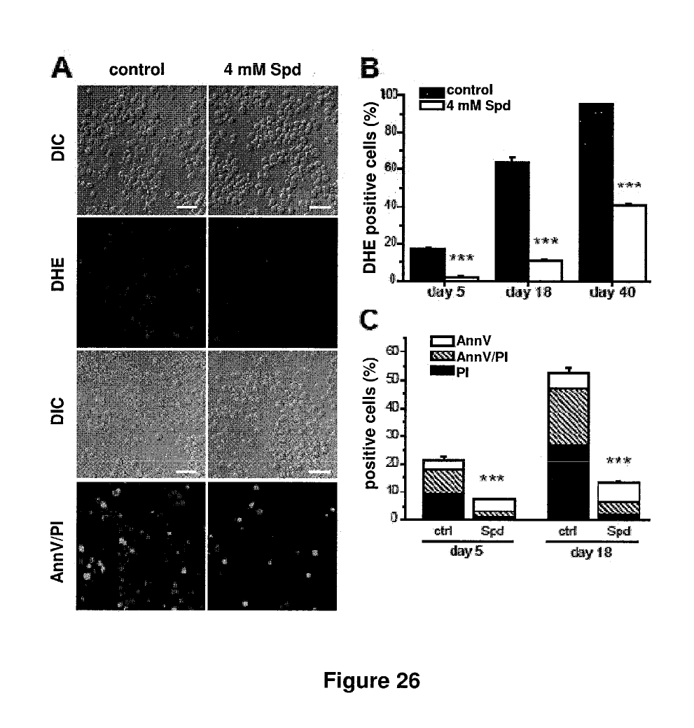

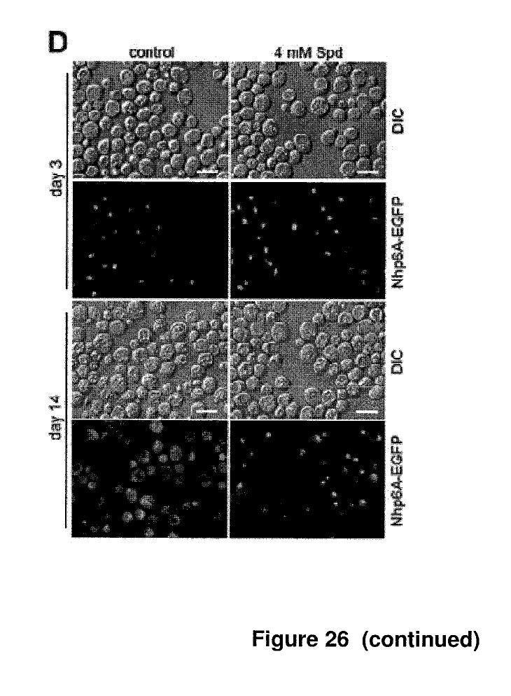

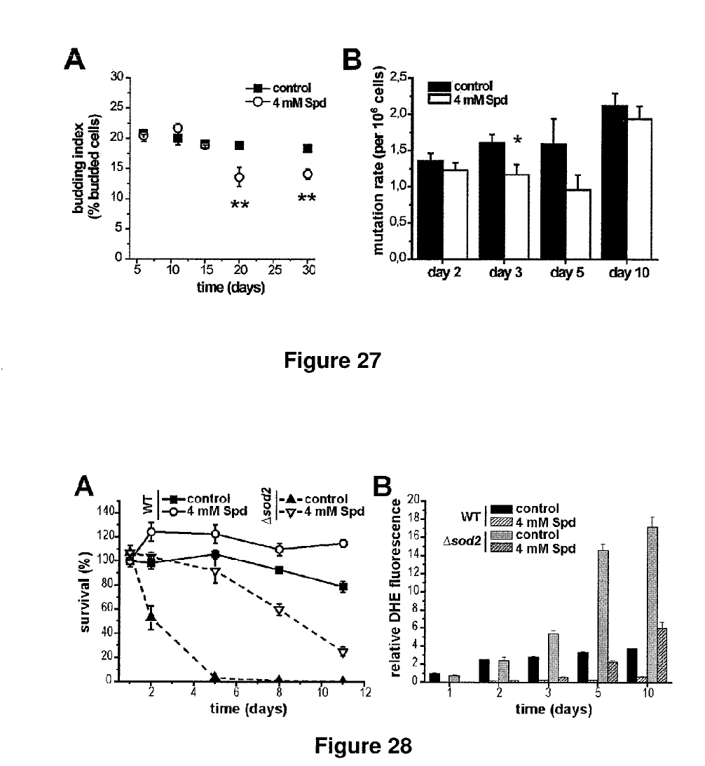

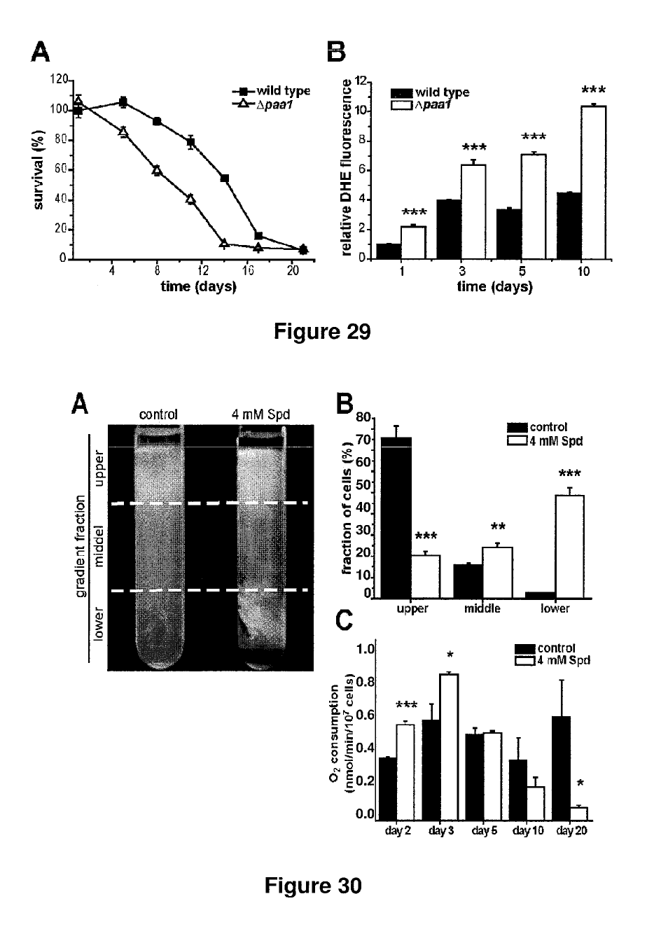

FIG. 27 shows life span extension by spermidine treatment is not due to regrowth of better adapted mutants. (A) Budding index of wild type cells at indicated time points during chronological aging with (∘) or without (▪) application of 4 mM spermidine, similar to the aging experiment as shown in FIG. 26A. Data represent means±SEM (n=3) with at least 500-1000 cells evaluated for each replicate. **p<0.01. (B) Mutation rate per 10⁶ living cells determined by canavanine resistance of wild type cells at indicated time points during chronological aging with (open bars) or without (closed bars) application of 4 mM spermidine, similar to the aging experiment as shown in FIG. 26A. Data represent means±SEM (n=5). *p<0.05.

FIG. 28 shows that spermidine application temporarily protects from excessive ROS accumulation and loss of survival in sod2 mutant cells during aging. (A) Survival during chronological aging of wild type (WT) and Δsod2 yeast with (open symbols) or without (closed symbols) application of 4 mM spermidine. Data represent means±SEM (n=4). (B) Quantification (fluorescence reader) of ROS accumulation using DHE staining of cells obtained from the aging experiment shown in (A). Data represent means±SEM (n=4).

FIG. 29 shows that deletion of the polyamine acetyltransferase PAA1 shortens chronological life span and enhances oxidative stress. (A) Survival during chronological aging of wild type and Δpaa1 yeast cells. Data represent means±SEM (n=4). (B) Quantification (fluorescence reader) of ROS accumulation using DHE staining of cells obtained from the aging experiment shown in (A). Relative fluorescence units have been normalized to wild type at day1. Data represent means±SEM (n=4). ***p<0.001.

FIG. 30 shows that spermidine treatment causes remodelling of chronologically aging cells into a low metabolic, quiescence-like state. (A) Sucrose gradient centrifugation of 22 day old wild type yeast chronologically aged with or without (control) treatment of 4 mM spermidine. A representative photograph is shown, picturing upper, middle, and lower (quiescent) cells. (B) Quantification of upper, middle, and lower fraction of cells after sucrose gradient centrifugation representatively shown in (A). Data represent means±SEM of three independent experiments. **p<0.01, ***p<0.001. (C) Oxygen consumption of wild type cells treated with or without (control) 4 mM spermidine during chronological aging. Oxygen consumption has been determined using O₂-electrode measurements and normalized to living cells (see Methods section). Data represent means±SEM (n=3). *p<0.05, ***p<0.001.

### DESCRIPTION OF ILLUSTRATIVE EMBODIMENTS

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

It is to be noticed that the term “comprising”, used in the claims, should not be interpreted as being restricted to the means listed thereafter; it does not exclude other elements or steps.

Reference throughout this specification to “one embodiment” or “an embodiment” means that a particular feature, structure or characteristic described in connection with the embodiment is included in at least one embodiment of the present invention. Thus, appearances, of the phrases “in one embodiment” or “in an embodiment” in various places throughout this specification are not necessarily all referring to the same embodiment, but may.

Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects.

The invention will now be described by a detailed description of several embodiments of the invention. It is clear that other embodiments of the invention can be configured according to the knowledge of persons skilled in the art without departing from the technical teaching of the invention, the invention being limited only by the terms of the appended claims.

The following terms are provided solely to aid in the understanding of the invention.

The term “pharmaceutically acceptable” is used adjectivally herein to mean that the compounds are appropriate for use in a pharmaceutical product. The term “physiologically acceptable” also means that the compounds are appropriate for use in a pharmaceutical product.

As used herein, the phrase “physiologically acceptable salts” or “pharmaceutically acceptable salts” or “nutraceutically acceptable salts” refers to salts prepared from pharmaceutically acceptable, preferably nontoxic, acids and bases, including inorganic and organic acids and bases, including but not limited to, sulfuric, citric, maleic, acetic, oxalic, hydrochloride, hydro bromide, hydro iodide, nitrate, sulfate, bisulfite, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e. 1,1′-methylene-bis-(2-hydroxy-3-naphthoate)) salts. Pharmaceutically acceptable salts include those formed with free amino groups such as, but not limited to, those derived from hydrochloric, phosphoric, acetic, oxalic, and tartaric acids. Pharmaceutically acceptable salts also include those formed with free carboxyl groups such as, but not limited to, those derived from sodium, potassium, ammonium, sodium lithium, calcium, magnesium, ferric hydroxides, isopropylamine, triethylamine, 2-ethylamino ethanol, histidine, and procaine.

As used herein, the term “carrier” refers to a diluent, adjuvant, excipient, or vehicle. Such carriers can be sterile liquids, such as saline solutions in water, or oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions.

As used herein, the term “mineral” refers to a substance, preferably a natural substance that contains calcium, magnesium or phosphorus. Illustrative nutrients and minerals include beef bone, fish bone, calcium phosphate, egg shells, sea shells, oyster shells, calcium carbonate, calcium chloride, calcium lactate, calcium gluconate and calcium citrate.

The term “treatment” refers to any process, action, application, therapy, or the like, wherein a mammal, including a human being, is subject to medical aid with the object of improving the mammal's condition, directly or indirectly.

In its broadest sense, the term a “critically ill patient” (herein designated CIP) refers to a patient who is experiencing an acute life-threatening episode or who is diagnosed to be in imminent danger of such an episode. A critically ill patient is medically unstable, and when not treated, likely to die.

The term critically ill patient refers to a patient who has sustained or is at risk of sustaining acutely life-threatening single or multiple organ system failure due to disease or injury, a patient who is being operated and where complications supervene, and a patient who has been operated in a vital organ within the last week or has been subject to major surgery within the last week.

In a more restricted sense, the term a “critically ill patient”, as used herein refers to a patient who has sustained or are at risk of sustaining acutely life-threatening single or multiple organ system failure due to disease or injury, or a patient who is being operated and where complications supervene.

In an even more restricted sense, the term a “critically ill patient”, as used herein refers to a patient who has sustained or are at risk of sustaining acutely life-threatening single or multiple organ system failure due to disease or injury. Similarly, these definitions apply to similar expressions such as “critical illness in a patient” and a “patient is critically ill”. A critically ill patient is also a patient in need of cardiac surgery, cerebral surgery, thoracic surgery, abdominal surgery, vascular surgery, or transplantation, or a patient suffering from neurological diseases, cerebral trauma, respiratory insufficiency, abdominal peritonitis, multiple trauma, severe burns, or critical illness polyneuropathy. The term “critical illness” as used herein refers to the condition of a “critically ill patient”. Critical illness induces swelling, enlargement and disfunction of mitochondria. In liver, but not in skeletal muscle, this is further aggrevated by excessive hyperglycemia.

The term “Intensive Care Unit” (herein designated ICU), as used herein refers to the part of a hospital where critically ill patients are treated. Of course, this might vary from country to country and even from hospital to hospital and the part of the hospital may not necessary, officially, bear the name “Intensive Care Unit” or a translation or derivation thereof. Of course, the term “Intensive Care Unit” also covers a nursing home, a clinic, for example, a private clinic, or the like if the same or similar activities are performed there. The term “ICU patient” refers to a “critically ill patient”.

The term “multiple organ dysfunction” or “multiple organ dysfunction syndrome” or “MODS” refers to a condition resulting from infection, injury (accident, surgery), hypoperfusion or hypermetabolism.

The “multiple organ failure” of which critically ill patients die, is considered a descriptive clinical syndrome, defined by a dysfunction or failure of at least two vital organ systems. The vital organ systems that are uniformly and most specifically affected are the liver, the kidneys, the lungs, as well as the cardiovascular system, the nervous system and the hematological system. In addition, skeletal muscle wasting and weakness contributes to failure to wean patients off from mechanical ventilation.

MODS comprises but is not limited to acute respiratory distress syndrome, heart failure, liver failure, renal failure, respiratory insufficiency, intensive care, shock and systemic inflammatory response syndrome. MODS is characterized by a progressive deterioration and subsequent failure of the body's physiological system. The primary cause triggers an uncontrolled inflammatory response. In operative and non-operative patients sepsis is the most common cause. Sepsis may result in septic shock. In the absence of infection a sepsis-like disorder is termed systemic inflammatory response syndrome (SIRS). Both SIRS and sepsis could ultimately progress to MODS. However, in one-third of the patients no primary focus can be found. MODS is well established as the final stage of a continuum ranging from SIRS to sepsis to severe sepsis to MODS. The terminology “enterally administering” encompasses oral administration (including oral gavage administration) as well as rectal administration, oral administration being most preferred. Unless indicated otherwise, the dosages mentioned in this application refer to the amounts delivered during a single serving or single administration event. If the present composition is ingested from a glass or a container, the amount delivered during a single serving or single administration will typically be equal to the content of the glass or container.

The term “parenterally administering” refers to delivery of substances given by routes other than the digestive tract, and covers administration routes such as intravenous, intraarterial, intramuscular, intracerebroventricular, intraosseous intradermal, intrathecal, and intraperitoneal administration and intravesical infusion and intracavernosal injection.

Typically “parenteral administration” refers to intravenous administration. A particular form of parenteral administration refers to the delivery by intravenous administration of nutrition (“parenteral nutrition”). Parenteral nutrition is called “total parenteral nutrition” when no food is given by other routes.

“Parenteral nutrition” is a isotonic or hypertonic aqueous solution (or solid compositions to be dissolved, or liquid concentrates to be diluted to obtain an isotonic or hypertonic solution) comprising a saccharide such as glucose and further comprising one or more of lipids, amino acids, and vitamins.

Particular and preferred aspects of the invention are set out in the accompanying independent and dependent claims. Features from the dependent claims may be combined with features of the independent claims and with features of other dependent claims as appropriate and not merely as explicitly set out in the claims.

Thus, the claims following the detailed description are hereby expressly incorporated into this detailed description, with each claim standing on its own as a separate embodiment of this invention.

The present invention discloses the surprising finding that polyamines have a beneficial effect on combating life-treating conditions in critically ill patients.

Whereas the use of polyamines to treat infectious disorders can be explained by the fact that polyamines bind to lipopolysacharides of bacteria, it was unexpected that polyamines have a therapeutic activity on life threatening conditions caused by non-infectious disorders.

The invention further discloses the surprising finding that polyamines have a beneficial effect even when a patient is already is at far-developed stage of a disorder in that the patient is a critically ill patient in a life threatening condition.

Hyvonen et al. and Rasanen et al. (cited above) discuss the use of polyamines in the prevention and treatment of pancreatitis. However Hynonen emphasizes the fact that after induction of the pancreatitis the treatment can be started when symptoms occur, but does not suggest or encourages to perform a treatment when the diseases is further developed into life-threatening conditions. As indicated in Hyvonen, pancreatitis can result into multiple organ failure due to systemic factors, leading to complications associated with high mortality. However the experiments performed by Vynonen in mice models of pancreatitis do not show or suggest the treatment of these animals in further developed stages of pancreatitis, let al. one suggest the treatment of multiple organ failure in other conditions apart from pancreatitis. Tzirogiannis et al. (2004) in Arch Toxicol. 78, 321-329, describe the treatement of acute liver injury with putrescine. Herein putrescine is administered 2, 5 and 8 hours after the injection of cadmium chloride and gives a protective effect. However putrescine administration at 12, 15 and 18 h after cadmium injection, when the disease becomes life threatening, did not provide a protective effect.

The invention further discloses the surprising finding that life threatening conditions can be alleviated or treated by metabolisable polyamines, i.e. polyamines with primary (NH2) or secondary (NH) amines. The prior art of Vynonen and Rasanen explain that a depletion in spermine and spermidine is caused by an enhanced activity of the acetylating enzyme SSAT (Spermine/Spermidine Actyl Transferase) leading to the degradation of the polyamine. The prior teaches that in order to treat pancreatitis, polyamines should be modified (e.g. methylated) at least one amine position to avoid acetylation of the enzymes. The findings of the present invention show that unmodified polyamines, which are substrates for the degradation via acetylation, are active indeed. Accordingly the teaching of the present invention allows using commercially available, natural compounds occurring in the human body, which are metabolized, whereas the modified compounds of Hyvonen and Hassane are foreign to the body and may accumulate in the body without being degraded.

The invention further discloses the surprising finding that the treatment with polyamines according to embodiments of the present invention can be combined with the administration of high nutrient compositions such as glucose comprising solutions for enteral or parenteral (e.g. intravenous) administration.

Vanhorebeek et al. (2005) Lancet 365, 53-59 discuss the detrimental effects of glucose in critically ill patients. The experiments performed in the present invention on in mice and model organisms such as yeast show that the addition of polyamines has a beneficial effect on critically ill patients who are at risk of nutrient overload or starvation. The present invention discloses that the removal of damaged cell organelles by autophagy acts as a cell protective mechanism and that this autophagy process functions better under caloric restricted conditions. These caloric restricted animals, who have a continuous stimulation of autophagy, are protected against age-related diseases and have a longer life span (Colman et al. (2009) Science 325, 201-204).

At the one hand, starvation in critically ill patients is not an option as these results in muscle degradation and severe weakness, and this ultimately causes respiratory pump failure, the inability to wean patients from mechanical ventilation, and thus precludes any rehabilitation. But also prolonged hypocaloric feeding can result in impaired outcome (Villet et al., (2005) Clin Nutr 24, 502-509.

On the other hand damaged organelles, which are even more abundantly present in humans and in animals who are in critically ill conditions (such as caused by trauma or complex injuries) need to be removed to preserve the cellular function in vital organs and systems. This removal, which is stimulated by autophagy which is considered to be an important defense process in critical, is inhibited by when the calorie restricted conditions are compensated by nutrient overload and hyperglycemia.

Although parenteral feeding has shown to reduce muscle wasting during critical illness, it is a powerful suppressor of autophagy in vital organs, and thus we hypothesized that autophagy is an important defense process in critical illness. Indeed, damaged organelles, which are even more abundantly present in patients and in animals who have undergone critical illness (induced by trauma or complex injuries) and who receive parenteral nutrition and hence are exposed to additional risks such as nutrient overload and hyperglycemia, need to be removed properly to preserve the cellular function in vital organs and systems. As such, feeding-induced suppression of autophagy could counteract any benefit obtained by feeding. Such a mechanism may explain why we found that starved animals have a better functioning of liver mitochondria, because damaged mitochondria are better removed by starvation-induced autophagy.

Without being bound by theory, the present experiments show that polyamines such as spermidine offer protection against cellular damage, which can be at least partially explained by reactivating autophagy, leading to, but not restricted to e.g. better functioning mitochondria (increased clearance of damaged mitochondria) and subsequent protection of vital organ systems. It has been found that these beneficial effects of polyamines abrogates vital organ dysfunction and lethality induced by parenteral feeding in critically ill animals, even in the presence of pronounced hyperglycemia. Accordingly the present invention illustrates that the suppression of autophagy caused by parental feeding is compensated by the administration of polyamines such as spermidine. Accordingly, particular embodiments of the present invention relate to a method of treating a life threatening condition in a critically ill human patient with a non-infectuous disorder comprising the step of administering an autophagy inducing agent to said patient. Apart from the above mentioned polyamines, suitable autophagy inducing agents include rapamycin, trehalose, resveratrol, and nicotinamide. Rapamycin analogues have been described in WO 93/11130, WO 94/02136, WO 94/02385, WO 95/14023, WO 94/09010 and WO 96/41807.

Different other autophagy inducing agents have been identified with assays such as disclosed in the examples in the present invention or with assays such as monodansylcadaverine (MDC) staining (Ravikumar et al. (2003) Hum. Mol. Gen. 12, 985-994, LC3 processing (Kabeya et al. (2000) EMBO J. 19, 5720-5728), or optical determination of autophagosome numbers. US2009049242 provides an assay to determine the level and localisation of LC3.

Apart from the mTOR pathway wherein rapamycin is involved, several other pathways have been identified which are involved in autophagy and where the modulators of this pathway can enhance autophagy. These disclosures equally disclose assays to determine whether a test compound has an autophagy inducing activity.

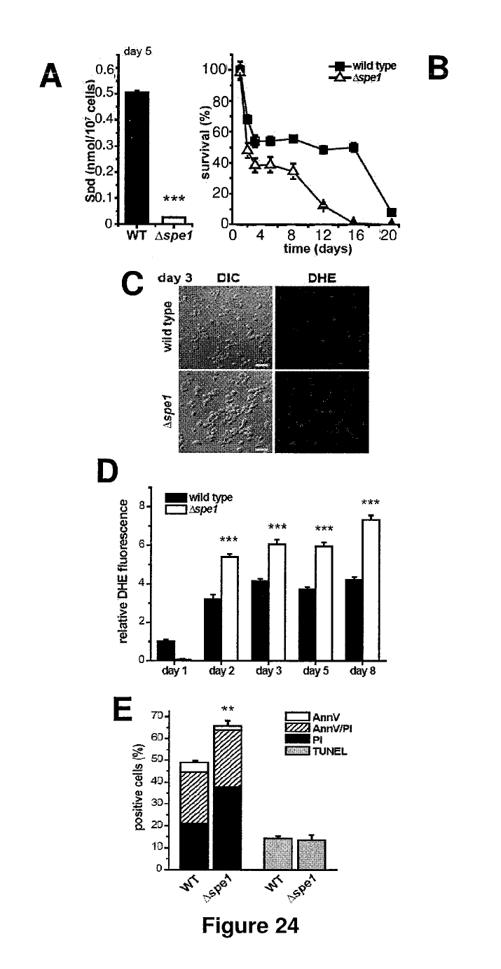

Sarkar et al. disclose in Nat Chem Biol (2007) 3, 331-338 and in WO/2008/122038 a group of compounds, so called “Small-Molecule Enhancers of Rapamycin (SMERs)”, which enhance autophagy. The chemical structure of exemplary compounds is shown in FIG. 24 of WO/2008/122038. This PCT application equally discloses methods to identify such SMER compounds.

Most SMER compounds induce autophagy via the TOR-pathway (similar to Rapamycin). A number of them also act independent of the TOR-pathway (SMERs 10, 18 and 28 in the above cited publications). The latter compounds can be of particular use for those patients where the TOR pathway is hyperactive and where rapamycin or structural analogues thereof can not further activate this pathway.

WO2007003941 discloses that calpain inhibitors induce autophagy. Calpain inhibitors include calpastatin (Wendt et al. (2004) Biol. Chem. 385, 465-472), ALLM, ALLN (Logie et. al. (2005) Mol Genet Metab. 85, 54-60), calpeptin (Ariyoshi et al. (1991) Biochem Int 23(S), 1019-33), leupeptin, α-dicarbonyls, quinolinecarboxamides, sulfonium methyl ketones, diazomethyl ketones, Leu-Abu-CONHEt (AK275), 27-mer calpastatin peptide and Cbz-Val-Phe-H (MDL28170) (Liu et al. (2004) Annu. Rev. Pharmacol. Toxicol. 44, 349-370). Suitable calpain inhibitors also include, for example, calpeptin (Z-Leu-Nle-H), α-mercaptoacrylic acids, phosphorus derivatives, epoxysuccinates, acyloxymethyl ketones, halomethylketones (Wang et al. (1997) Adv. Pharmacol. 37, 117-152) and E64 (EST) (Gollet al. (2003) Physiol. Rev. 83, 731-801).

WO2006079792 discloses that inhibitors of inositol monophosphatase (IMPase) induce autophagy. IMPase inhibitors include L-690330, L-690488 (Atack et al. (1994) J Pharmacol Exp Ther. 270, 70-76), lithium, valproate and carbemazapine. Other examples of suitable IMPase inhibitors are described in Fauroux (1999) Enzyme Inhib. 14, 97-108; Miller (2004) Org Biomol Chem. 2, 671-88. and Atack (1994) J Pharmacol Exp Ther. 270, 70-76. IMPase inhibitors include inositol-1-monophosphate analogues or variants, (Fauroux (1999) J Enzyme Inhib. 14, 97-108. IMPase inhibitors also include bisphosphonates, such as 1-hydroxyethylidene-1,1 bisphosphonic acid and hydroxymethylene isphosphonic acid, terpenoids such as sesquiterpene L-671776 from Memnoniella echinata and puberulonic acid from Penicillum spp and tropolones, in particular hydroxyl substituted tropolones such as 7-hydroxytropolone and 3,7-dihydroxytropolone and analogues, derivatives and salts thereof.

WO2008099175 discloses that inhibiting or reducing the activity of the cAMP/EPAC/PLC pathways induces autophagy. cAMP antagonists include clonidine, rilmendine, tyramine morphine, baclofen, G protein receptor-derived peptides (Taylor et al. (1994) Cell Signal 6, 841-849), mastoparan (Higashijima et al. (1988) J. Biol Chem 263, 6491-6494; Higashijima et al. (1990) J. Biol. Chem. 265, 14176-14186), propranolol, bupivacain (Hageluken et al. (1994) Biochem. Pharmacol. 47, 1789-1795), quinine, aspartame (Nairn et al. (1994) Biochem. J. 297, 451-454), N-dodecyl lysinamide and FUB 86 (Leschke et al. (1997) J. Med. Chem. 40, 3130-3139; Breitweg-Lehmann (2002). Mol. Pharmacol. 61, 628-636; Mousli et al. (1990) Trends Pharmacol. Sci. 11, 358-362).

Other autophagy inducing agents interfering with this pathway include Gsα and its ligand, pituitary adenylate cyclase-activating polypeptide (PACAP) or other members of the PACAP/Glucagon superfamily. Members of the PACAP/Glucagon superfamily include secretin, peptide histidine methionine (PHM), vasoactive intestinal peptide (VIP), glucagon, glucagon like peptide-1 (GLP-I), GLP-2, glucose dependent insulinotropic polypeptide (GIP), GH releasing factor (GRF) and PACAP related peptide (PRP) (Sherwood et al. (2000) Endocrine reviews 21, 619-70).

Other autophagy inducing agents interfering with this pathway include suramin and suramin analogues, such as NF449 and NF503.

WO2010018182 discloses peptide analogues which stimulate autophahy. These peptides comprise a Thr-Gln-Thr amino acid triplet followed by at least 5 amino acid residues forming an α-helix secondary structure.

The present invention discloses an animal model of prolonged critical illness that mimics the human condition. Indeed, these critically ill animals undergo the same metabolic, immunological and endocrine disturbances and development of organ failure and muscle wasting as the human counterpart. In this animal model, parenteral feeding has an effect on the overall outcome of the animals. Compared to starvation, a small dose of parenteral feeding in critically ill animals decreased muscle catabolism and did not induce significant lethality. A higher dose of parenteral feeding however holds risk of death, which thus reflects a trade-off for improved muscle preservation. As soon as hyperglycemia is allowed to develop, a higher lethality precludes any benefit from parenteral feeding. Indeed, parenteral feeding has also disadvantages, one of which is development of hyperglycemia, which, if left untreated, leads to increased mortality, multiple organ failure and muscle breakdown. Even brief cellular hyperglycemia and nutrient overload exerts direct toxic cellular effects in the setting of critical illness, leading to these disastrous effects. Prevention of hyperglycemia in the critically ill, however, is difficult to achieve, specifically since there is a risk of hypoglycemia, which could counteract any benefit.

The present invention illustrates that such lethal effects of parental feeding in critically ill animals can be abrogated by administration of polyamines such as spermidine.

The present invention describes polyamines, compositions comprising polyamines and their use in the treatment of life threatening conditions in critically ill patients.

Polyamines are generally described as basic, water soluble, low molecular weight aliphatic molecules with two (diamines) or more amine groups.

Particular amines in the context of the present invention are diamines represented by the general formula NH₂—(CH₂)₂₋₁₀—NH₂, which are unsubstituted at the carbon atoms or wherein one or more carbon atoms are optionally substituted with a methyl group, an NH or oxygen. The diamine group of polyamines comprises ethylene diamine, 1,3 diaminopropane, 1,4 diaminobutane (putrescine), 1,5 diaminopentane (cadaverine), 1,6-diamino-hexane, 1,7-diamino-heptane and 1,8-diamino-octane. Particular diamines in the context of the present invention are 1,4 diaminobutane (putrescine) and 1,5 diaminopentane (cadaverine), more particularly are 1,4 diaminobutane (putrescine) Other particular polyamines have a general structure NH₂—((CH₂)ₘ—NH)ₙ—H, wherein m and n are each independently integers from 2 to 6. These polyamines are typically unsubstituted at the carbon atoms. Optionally one or more carbon atoms are substituted with a methyl group, and/or NH and/or oxygen group.

In particular embodiments m is 3, 4 or 5, more particularly 4. In particular embodiments n is 3 or 4. Particular polyamines are spermine H₂N((CH₂)₄—NH)₃—H (m is 4 and n is 3) and spermidine NH₂((CH₂)₄—NH)₂H (m is 4 and n is 2).

More particular embodiments in the context of the present invention are polyamines which are metabolisable, i.e. in that the polyamines are a substrate for the acetylating enzyme SSAT. According to this embodiment, polyamines which are methylated at one or more NH₂ or NH groups are disclaimed.

Alternatively, according to an alternative embodiment polyamines can be acetylated at one or more of NH₂ or NH groups.

The present invention relates in particular embodiments to a polyamine compound of the group consisting of putrescine (1,4-diamino-butane), 1,3-diamino-propane, 1,7-diamino-heptane, 1,8-diamino-octane, spermine, spermidine, or a derivative thereof or a pharmaceutically acceptable salt, solvate or isomer thereof, such as or combinations thereof, for use in a treatment of treating or preventing multiple organ dysfunction in a critically ill patient.

In one embodiment, the polyamine compound of present invention is spermidine or a spermidine analog of the group consisting of spermidine phosphate hexahydrate, spermidine phosphate hexahydrate and L-arginyl-3,4-spermidine or a pharmaceutically acceptable salt, solvate or isomer thereof, or combinations thereof.

In a particular embodiment, the polyamine compound of present invention is a polyamine compound of the group consisting of putrescine, spermine, and spermidine.

In a preferred embodiment, the polyamine compound of present invention is spermidine or a pharmaceutically acceptable salt, solvate or isomer thereof, or combinations thereof.

In embodiments of the present invention, the polyamine compound is used in a treatment of treating or preventing multiple organ dysfunction in a critically ill patient wherein the multiple organ dysfunction is not caused or associated with sepsis.

The polyamine compound can be used in a treatment of multiple organ dysfunction wherein the polyamine compound is administered parenterally or enterally to the critically ill patient, or is administered by a bolus injection, e.g. an intravenous bolus injection.

In a preferred embodiment, the polyamine compound of present invention is used in a treatment of multiple organ dysfunction wherein the critically ill patient further receives total parenteral nutrition.

In another preferred embodiment, the polyamine compound of present invention is used in a treatment of multiple organ dysfunction in a critically ill patient receiving parenteral nutrition.

In yet another preferred embodiment, the polyamine compound of present invention is used in a treatment of multiple organ dysfunction in a critically ill patient with failed or disturbed homeostasis receiving parenteral nutrition.

In a particular embodiment, the polyamine compound of present invention is used in a treatment to protect a critically ill patient against multiple organ dysfunction by inducing adipocytes dedifferentiation.

In a particular embodiment, the polyamine compound of present invention is used in a treatment to protect a critically ill patient against multiple organ dysfunction by inducing dedifferentiation of adipocytes, e.g. inducing dedifferentiation of adipocytes into new into adipogenic, chondrogenic and osteogenic lineages, which results in reduced size of adipocytes and increased adipose mass.

Mature, lipid-containing adipocytes possess the ability to undergo symmetrical or asymmetrical cell division by a process called dedifferentiation of adipocytes. Such dedifferentiated adipocytes can function as seed cells and are capable of further differentiating into adipogenic, chondrogenic and osteogenic lineages. Such small adipocytes have been observed in patients during the course of critical illness. This process of adipocyte dedifferentiation and the formation of new adipocytes from the seed/precursor cells can be further be enhanced by a treatment with a polyamine compound of the group consisting of putrescine (1,4-diamino-butane), 1,3-diamino-propane, 1,7-diamino-heptane, 1,8-diamino-octane, spermine, or a derivative thereof or a pharmaceutically acceptable salt, solvate or isomer thereof, such as spermidine, cholesteryl spermine, spermidine trihydrochloride, spermidine phosphate hexahydrate, spermidine phosphate hexahydrate, and 1,4-butanediamine N-(3-aminopropyl)-monohydrochloride or combinations thereof. Such induced dedifferentiation of adipocytes and further differentiation of the seed cells turn adipose tissue into a functional ‘waist bin’ for toxic metabolites such as glucose during critical illness and is protective against multiple organ dysfunction in a critically ill patient.

A preferred embodiment of present invention is spermidine or a spermidine analogue of the group consisting of spermidine phosphate hexahydrate, spermidine phosphate hexahydrate and L-arginyl-3,4-spermidine or a pharmaceutically acceptable salt, solvate or isomer thereof, or combinations thereof for use in a treatment to protect a critically ill patient against toxic metabolites by enhance dedifferentiation of adipocytes and of absorption of toxic metabolites in the adipose tissue protection of a critically ill patient.

A preferred embodiment of present invention is spermidine or a spermidine analogue of the group consisting of spermidine phosphate hexahydrate, spermidine phosphate hexahydrate and L-arginyl-3,4-spermidine or a pharmaceutically acceptable salt, solvate or isomer thereof, or combinations thereof for use in a treatment to protect a critically ill patient against multiple organ dysfunction by enhance dedifferentiation of adipocytes and of absorption of toxic metabolites in the adipose tissue protection of a critically ill patient.

A polyamine compound of the group consisting of putrescine (1,4-diamino-butane), 1,3-diamino-propane, 1,7-diamino-heptane, 1,8-diamino-octane, spermine, spermidine, or a derivative thereof or a pharmaceutically acceptable salt, solvate or isomer thereof, such as cholesteryl spermine, spermidine trihydrochloride, spermidine phosphate hexahydrate, spermidine phosphate hexahydrate, and 1,4-butanediamine N-(3-aminopropyl)-monohydrochloride or combinations thereof, for use in a treatment of to induce or enhance the dedifferentiation of adipocytes and absorption of toxic metabolites into the protection of the critically ill patent against toxic metabolites.

The present invention also relates to a pharmaceutical composition comprising a pharmacologically acceptable amount of a polyamine compound of the group consisting of putrescine, 1,4-diamino-butane, 1,3-diamino-propane, 1,7-diamino-heptane, 1,8-diamino-octane, spermine, spermidine, or a derivative thereof or a pharmaceutically acceptable salt, solvate or isomer thereof, or combinations thereof, such as cholesteryl spermine, spermidine trihydrochloride, spermidine phosphate hexahydrate, spermidine phosphate hexahydrate, and 1,4-butanediamine N-(3-aminopropyl)-monohydrochloride for use in a treatment of treating or preventing multiple organ dysfunction in a critically ill patient.

In one embodiment, the pharmaceutical composition of present invention comprises a pharmacologically acceptable amount of a polyamine compound of the group consisting of cholesteryl spermine, spermidine trihydrochloride, spermidine phosphate hexahydrate, spermidine phosphate hexahydrate, and 1,4-butanediamine N-(3-aminopropyl)-monohydrochloride or a derivative thereof or a pharmaceutically acceptable salt, solvate or isomer thereof, or combinations thereof.

In a particular embodiment, the pharmaceutical composition of present invention comprises a polyamine compound which is spermidine or a spermidine analog of the group consisting of spermidine phosphate hexahydrate, spermidine phosphate hexahydrate and L-arginyl-3,4-spermidine or a pharmaceutically acceptable salt, solvate or isomer thereof, or combinations thereof.

In a preferred embodiment, the pharmaceutical composition of present invention comprises a polyamine compound, which is a pharmacologically acceptable amount of spermidine or a pharmaceutically acceptable salt, solvate or isomer thereof, or combinations thereof.

In one embodiment, the pharmaceutical composition of present invention comprises the polyamine compound of present invention in the range of about 0.05% to about 4% of the aqueous liquid composition.

In one embodiment, the pharmaceutical composition of present invention comprises the polyamine compound in the range of about 0.5% to about 2% of the aqueous liquid composition.

In one embodiment, the pharmaceutical composition of present invention comprises the polyamine compound in the range of about 1.0% to about 1.5% of the aqueous liquid composition.

In another embodiment, the pharmaceutical composition of present invention further comprises a pharmaceutically acceptable carrier or a blood glucose regulator or nutrients, e.g. essential nutrients.

The pharmaceutical composition can be provided as an aqueous liquid composition. Moreover, the pharmaceutical composition can be administered parenterally or enterally to the critically ill patient, or is administered by a bolus injection, e.g. an intravenous bolus injection. In a preferred embodiment, the critically ill patient further receives total parenteral nutrition.

The pharmaceutical composition can be provided to normalize the plasma spermidine level in the critically ill patient, or to augment the plasma spermidine level in the critically ill patient to a level that is 1 to 2.5, 4 or even 5 times the plasma spermidine level of a healthy person with a similar body weight as the critically ill patient. In an embodiment, the pharmaceutical composition can be provided to augment the plasma spermidine level in the critically ill patient to a level that is twice the plasma spermidine level of a healthy person with a similar body weight as the critically ill patient. Also, the pharmaceutical composition of present invention is for use in a treatment to augment the plasma spermidine level in the critically ill patient to a level in the range of 50 to 3500 or 6000 nmol/l plasma.

In one embodiment, the pharmaceutical composition of present invention is for use in a treatment to augment the plasma spermidine level in the critically ill patient to a level that is restoring the plasma spermidine level to that of a healthy person with a similar body weight as the critically ill patient.

In another embodiment, the pharmaceutical composition of present invention is for use in a treatment to augment the plasma spermidine level in the critically ill patient to a level in the range of 50 to 3500 nmol/l plasma.

In another embodiment, the pharmaceutical composition of present invention is for use in a treatment to augment the plasma spermidine level in the critically ill patient to a level in the range of 100 to 6000 nmol/l plasma.

In another embodiment, the pharmaceutical composition of present invention is for use in a treatment to augment the plasma spermidine level in the critically ill patient by administering daily the polyamine compound in the weight range of 0.01 μg per kg to 100 mg per kg body weight.

In a preferred embodiment, the pharmaceutical composition of present invention is for use in a treatment of treating or preventing multiple organ dysfunction in a critically ill patient that is not caused or associated with sepsis.

In a preferred embodiment, the pharmaceutical composition of present invention is for use in a treatment to protect a critically ill patient against multiple organ dysfunction by inducing dedifferentiation of adipocytes, e.g. inducing dedifferentiation of adipocytes into new into adipogenic, chondrogenic and osteogenic lineages, which results in reduced size of adipocytes and increased adipose mass.

In a particular embodiment, the pharmaceutical composition of present invention is for use in a treatment to protect a critically ill patient against toxic metabolites by enhancing dedifferentiation of adipocytes and of absorption of toxic metabolites in the adipose tissue protection of a critically ill patient.

In a particular embodiment, the pharmaceutical composition of present invention is for use in a treatment to induce or enhance the dedifferentiation of adipocytes and absorption of toxic metabolites into the protection of the critically ill patent against toxic metabolites.

The present invention also relates to a composition that can be reconstituted with water to the pharmaceutical composition of present invention.

The present invention also relates to a method to treat or to prevent multiple organ dysfunction in a critically ill patient by administering to the critically ill patient a pharmaceutical composition comprising a pharmacologically acceptable amount of a polyamine compound of the group consisting of putrescine (1,4-diamino-butane), 1,3-diamino-propane, 1,7-diamino-heptane, 1,8-diamino-octane, spermine, spermidine, or a derivative thereof or a pharmaceutically acceptable salt, solvate or isomer thereof, such as cholesteryl spermine, spermidine trihydrochloride, spermidine phosphate hexahydrate, spermidine phosphate hexahydrate, and 1,4-butanediamine N-(3-aminopropyl)-monohydrochloride or combinations thereof, for use in a treatment of treating or preventing multiple organ dysfunction in a critically ill patient.

In one embodiment, the method to treat or to prevent multiple organ dysfunction in a critically ill patient comprises the step of administering to the critically ill patient a pharmaceutical composition comprising a pharmacologically acceptable amount of a polyamine compound of the group consisting of cholesteryl spermine, spermidine trihydrochloride, spermidine phosphate hexahydrate, spermidine phosphate hexahydrate, and 1,4-butanediamine N-(3-aminopropyl)-monohydrochloride or a derivative thereof or a pharmaceutically acceptable salt, solvate or isomer thereof, or combinations thereof.

In a particular embodiment, the method to treat or to prevent multiple organ dysfunction in a critically ill patient comprises the step of administering to the critically ill patient a pharmaceutical composition comprising a polyamine compound which is spermidine or a spermidine analog of the group consisting of spermidine phosphate hexahydrate, spermidine phosphate hexahydrate and L-arginyl-3,4-spermidine or a pharmaceutically acceptable salt, solvate or isomer thereof, or combinations thereof

In a preferred embodiment, the method to treat or to prevent multiple organ dysfunction in a critically ill patient comprises the step of administering to the critically ill patient a pharmaceutical composition comprising a polyamine compound which is a pharmacologically acceptable amount of spermidine or a pharmaceutically acceptable salt, solvate or isomer thereof, or combinations thereof.

In another preferred embodiment, the method to treat or to prevent multiple organ dysfunction in a critically ill patient comprises the step of administering to the critically ill patient a pharmaceutical composition that further comprises a pharmaceutically acceptable carrier and/or a blood glucose regulator and/or nutrients, e.g. essential nutrients. The pharmaceutical composition used in the method of present invention can be an aqueous liquid composition. The pharmaceutical composition can comprise the polyamine compound of present invention in the range of about 0.05, 0.1, 0.2 or 0.5% to about 1, 2, 3 or 4% of the aqueous liquid composition. The pharmaceutical composition can comprise the polyamine compound of present invention in the range of about 0.5% to about 2% of the aqueous liquid composition.

The pharmaceutical composition can comprise the polyamine compound of present invention in the range of about 1.0% to about 1.5% of the aqueous liquid composition. The method of present invention can normalize the plasma spermidine level in the critically ill patient. The method of present invention can augment the plasma spermidine level in the critically ill patient to a level that is twice the plasma spermidine level of a healthy person with a similar body weight as the critically ill patient. The method of present invention can augment the plasma spermidine level in the critically ill patient to a level in the range of 50-6000 nmol/l plasma, or can augment the plasma spermidine level in the critically ill patient by administering daily the polyamine compound in the weight range of 0.01, 0.05, 0.1, 0.2 or 0.5 to 1, 10, 20, 30 or 100 mg per kg body weight. Multiple organ dysfunction can thus be treated or prevented in a critically ill patient, for instance multiple organ dysfunction that is not caused or associated with sepsis.

The polyamine compound can be administered parenterally or enterally to the critically ill patient, or is administered by a bolus injection, e.g. an intravenous bolus injection. The critically ill patient can further receive total parenteral nutrition.

The pharmaceutical composition can be provided as an aqueous liquid composition. Moreover, it is advantageous of the method of present invention that the pharmaceutical composition can be administered parenterally or enterally to the critically ill patient. In a preferred embodiment, the critically ill patient further receives total parenteral nutrition.

The pharmaceutical composition can be provided to normalize the plasma spermidine level in the critically ill patient.

The pharmaceutical composition can be provided to augment the plasma spermidine level in the critically ill patient to a level that is 1 to 2.5, 4 or even 5 times the plasma spermidine level of a healthy person with a similar body weight as the critically ill patient. In an embodiment, the pharmaceutical composition can be provided to augment the plasma spermidine level in the critically ill patient to a level that is twice the plasma spermidine level of a healthy person with a similar body weight as the critically ill patient.

In one embodiment, the method of present invention can augment the plasma spermidine level in the critically ill patient to a level in the range of 50 to 6000 nmol/l plasma.

In one embodiment, the method of present invention can augment the plasma spermidine level in the critically ill patient to a level that is restoring the plasma spermidine level to that of a healthy person with a similar body weight as the critically ill patient.

In one embodiment, the method of present invention can augment the plasma spermidine level in the critically ill patient to a level in the range of 50 to 6000 nmol/1 plasma.

In one embodiment, the method of present invention can augment the plasma spermidine level in the critically ill patient to a level in the range of 100 to 6000 nmol/l plasma.

In one embodiment, the method of present invention can augment the plasma spermidine level in the critically ill patient by administering daily the polyamine compound in the weight range of 0.01 to 100 mg per kg body weight.

In one embodiment, the method of present invention can treat or prevent multiple organ dysfunction in a critically ill patient that is not caused or associated with sepsis.

In one embodiment, the method of present invention can protect a critically ill patient against multiple organ dysfunction by inducing dedifferentiation of adipocytes, e.g. inducing dedifferentiation of adipocytes into new into adipogenic, chondrogenic and osteogenic lineages, which results in reduced size of adipocytes and increased adipose mass.

In one embodiment, the method of present invention can protect a critically ill patient against toxic metabolites by enhancing dedifferentiation of adipocytes and of absorption of toxic metabolites in the adipose tissue protection of a critically ill patient.

In one embodiment, the method of present invention can induce or enhance the dedifferentiation of adipocytes and absorption of toxic metabolites into the protection of the critically ill patent against toxic metabolites.

In one embodiment, the method is used to treat a patient who has been diagnosed as having a paradoxal muscle waste syndrome.

In a particular embodiment, the method is used to treat an animal under a starvation condition, or a critically ill animal, or a critically ill patient. In a particular embodiment, the animal is a fasting animal such as a fasting mammal, more in particular a fasting human.

In a particular embodiment, the method is used to prevent or treat excessive catabolism in a critically ill patient or to reduce morbidity or mortality due to excessive catabolism in a critically ill patient. The treatment can particularly be applied to prevent loss of lean body mass due to critical illness or to prevent mortality due to significant loss of lean body mass in a critically ill patient. Moreover, the treatment is particularly used to induce adipocyte dedifferentiation by a direct action of the polyamine compound on adipocytes for bringing about beneficial, adaptive changes within the adipose tissue in a critically ill patient, which could be life-saving.

In another particular embodiment of the invention, the method is used to improve the nitrogen balance in a critically ill patient. The treatment can particularly be applied to increase lean body mass in a critically ill patient. Moreover, the treatment is particularly used to decrease length of time spent on ventilator in a critically ill patient.

In a particular embodiment, the method is used to induce a positive nitrogen balance and lean body mass in an animal in need thereof.

In a particular embodiment, the method is used to induce adipocyte dedifferentiation for reducing the size of adipocytes and to induce beneficial, adaptive changes within the adipose tissue in a critically ill patient, which could be life-saving.

In another particular embodiment of the invention, the method is used to treat a lipid disorder or a dyslipidemia.

The present invention further relates to the use of a polyamine compound of the group consisting of putrescine, (1,4-diamino-butane), 1,3-diamino-propane, 1,7-diamino-heptane, 1,8-diamino-octane, spermine, spermidine, or a derivative thereof or a pharmaceutically acceptable salt, solvate or isomer thereof, such as cholesteryl spermine, spermidine trihydrochloride, spermidine phosphate hexahydrate, spermidine phosphate hexahydrate, and 1,4-butanediamine N-(3-aminopropyl)-monohydrochloride or combinations thereof, to manufacture a medicament to treat or prevent multiple organ dysfunction in a critically ill patient.

In one embodiment, the use of the polyamine compound of present invention is the use of spermidine or a spermidine analog of the group consisting of spermidine phosphate hexahydrate, spermidine phosphate hexahydrate and L-arginyl-3,4-spermidine or a pharmaceutically acceptable salt, solvate or isomer thereof, or combinations thereof.

In a particular embodiment, the use of the polyamine compound of present invention is the use of a polyamine compound of the group consisting of putrescine, spermine, and spermidine.

In a preferred embodiment, the use of the polyamine compound of present invention is the use of spermidine or a pharmaceutically acceptable salt, solvate or isomer thereof, or combinations thereof.

In embodiments of the present invention, the polyamine compound is used to manufacture a medicament to treat or prevent multiple organ dysfunction in a critically ill patient wherein the multiple organ dysfunction is not caused or associated with sepsis.

The polyamine compound is used to manufacture a medicament to treat or prevent multiple organ dysfunction wherein the polyamine compound is administered parenterally or enterally to the critically ill patient, or is administered by a bolus injection, e.g. an intravenous bolus injection.

In a preferred embodiment, the polyamine compound of present invention is used to manufacture a medicament to treat or prevent multiple organ dysfunction wherein the critically ill patient further receives total parenteral nutrition.

In another preferred embodiment, the polyamine compound of present invention is used to manufacture a medicament to treat or prevent multiple organ dysfunction in a critically ill patient receiving parenteral nutrition.

In yet another preferred embodiment, the polyamine compound of present invention is used to manufacture a medicament to treat or prevent multiple organ dysfunction in a critically ill patient with failed or disturbed homeostasis receiving parenteral nutrition.

In a particular embodiment, the polyamine compound of present invention is used to manufacture a medicament to treat or prevent multiple organ dysfunction by inducing adipocytes dedifferentiation.

In a particular embodiment, the polyamine compound of present invention is used to manufacture a medicament to induce dedifferentiation of adipocytes.

In a particular embodiment, the polyamine compound of present invention is used to manufacture a medicament to induce dedifferentiation of adipocytes into new into adipogenic, chondrogenic and osteogenic lineages.

In a particular embodiment, the polyamine compound of present invention is used to manufacture a medicament to induce dedifferentiation of adipocytes resulting in reduced size of adipocytes and increased adipose mass.

In a particular embodiment, the polyamine compound of present invention is used to manufacture a medicament to protect a critically ill patient against toxic metabolites by enhancing dedifferentiation of adipocytes and of absorption of toxic metabolites in the adipose tissue protection of a critically ill patient.

In a particular embodiment, the polyamine compound of present invention is used to manufacture a medicament to induce or enhance the dedifferentiation of adipocytes and absorption of toxic metabolites into the protection of the critically ill patent against toxic metabolites.

Examples of trauma that can lead to MODS that can be treated prophylactically with the present method include surgery and major injuries such as burns, lesions and haemorrhage. The present method is particularly suitable for preventing MODS resulting from surgery, particularly prescheduled surgery. In case of, for instance, prescheduled surgery it is possible to administer the present liquid composition prior to the occurrence of the trauma Administration of the liquid composition prior to the occurrence of the trauma offers the important advantages that the composition can be administered simply by asking the patient to drink it and that the effect will be manifest when the actual trauma occurs.

Usually and preferably, the treatment of a critical ill patent necessitates prolonged minute-to-minute therapy and/or observation, usually and preferably in an intensive care unit (ICU) or a special hospital unit, for example, a post operative ward or the like which is capable of providing a high level of intensive therapy in terms of quality and immediacy.

According to particular embodiments of the present invention, the critically ill patient is selected from the group consisting of a patient in need of cardiac surgery, a patient in need of thoracic surgery, a patient in need of abdominal surgery, a patient in need of vascular surgery, a patient in need of transplantation, a patient suffering from neurological diseases, a patient suffering from cerebral trauma, a patient suffering from respiratory insufficiency, a patient suffering from abdominal peritonitis, a patient suffering from multiple trauma, a patient suffering from severe burns, a patient suffering from critical illness polyneuropathy (CIPNP) and a patient being mechanically ventilated.

In a further preferred embodiment of the present invention, the critically ill patient is a patient suffering from multiple organ dysfunction syndrome (MODS). Patients with life threatening illness are cared for in hospitals in the intensive care unit (“ICU”). These patients may be seriously injured from automobile accidents, etc., have had major surgery, have suffered a heart attack, or may be under treatment for cancer, or other major disease. While medical care for these primary conditions is sophisticated and usually effective, a significant number of patients in the ICU will not die of their primary disease. Rather, a significant number of patients in the ICU die from a secondary complication known commonly as “multiple organ failure”. The medical terms for the general terms “sepsis” and “septic shock” are systemic inflammatory response syndrome (“SIRS”), multiple organ dysfunction syndrome (“MODS”), and multiple organ system failure (“MOSF”) (collectively “SIRS/MODS/MOSF”).

Medical illness, trauma, complication of surgery, and, for that matter, any human disease state, if sufficiently injurious to the patient, may elicit SIRS/MODS/MOSF. The systemic inflammatory response within certain physiologic limits is beneficial. As part of the immune system, the systemic inflammatory response promotes the removal of dead tissue, healing of injured tissue, detection and destruction of cancerous cells as they form, and mobilization of host defenses to resist or to combat infection. If the stimulus to the systemic inflammatory response is too potent, such as massive tissue injury or major microbial infection, however, then the systemic inflammatory response may cause symptoms which include fever, increased heart rate, and increased respiratory rate. This symptomatic response constitutes SIRS. If the inflammatory response is excessive, then injury or destruction to vital organ tissue may result in vital organ dysfunction, which is manifested in many ways, including a drop in blood pressure, deterioration in lung function, reduced kidney function, and other vital organ malfunction. This condition is known as MODS. With very severe or life threatening injury or infection, the inflammatory response is extreme and can cause extensive tissue damage with vital organ damage and failure. These patients will usually die promptly without the use of ventilators to maintain lung ventilation, drugs to maintain blood pressure and strengthen the heart, and, in certain circumstances, artificial support for the liver, kidneys, coagulation, brain and other vital systems. This condition is known as MOSF. These support measures partially compensate for damaged and failed organs; they do not cure the injury or infection or control the extreme inflammatory response which causes vital organ failures.

Because no effective treatments have been developed so far, MODS is associated with high mortality rates. MODS is no longer viewed as a series of isolated failures. On autopsy, the involved organs display similar patterns of tissue damage although they are often remote from the initial injury site or septic source. This complex syndrome, once thought to be solely related to cardiovascular dysfunction and/or isolated organ failure, is now recognized as a systemic disturbance mediated by a sustained inflammatory response to injury, regardless of the initiating factor(s). MODS attests to the complex interaction between organ systems in both their functioning and pathological states.

Several mechanisms have been postulated to be involved in post-ischemia induction of MODS. The gut-liver-lung axis has been associated to play a dominant role in the incidence and severity of this single and multiple organ dysfunction syndrome (S)MODS. More specifically, the intestine is often referred to as the driving force of MODS. The post-ischemic increase in reactive oxygen species can directly or indirectly (by macrophages and lymphocytes) activate neutrophils that subsequently can infiltrate at the site of inflammation causing tissue injury. These neutrophils have recently also been reported to increase paracellular transport in ileum. This damage of the intestinal barrier has often been mentioned to result in increased trans-epithelial bacterial transport and their endotoxins resulting in an inflammatory challenge of the patient, which has been reported to be involved in the incidence of MODS.

In a specific embodiment, the polyamine of present invention is spermine or spermidine.

When administered to a patient, a polyamine compound is preferably administered as a component of a composition that optionally comprises a pharmaceutically acceptable carrier or vehicle. In one embodiment, these compositions are administered orally. In a preferred embodiment, the polyamine compound of present invention is a component of a pharmaceutical composition that is administered intravenously.

A pharmaceutical composition comprising a polyamine compound of present invention can be administered via one or more routes such as, but not limited to, oral, intravenous infusion, subcutaneous injection, intramuscular, topical, depo injection, implantation, time-release mode, and intracavitary. The pharmaceutical composition is formulated to be compatible with its intended route of administration. Examples of routes of administration include parenteral, e.g., intravenous, intramuscular, intraperitoneal, intracapsular, intraspinal, intrasternal, intratumor, intranasal, epidural, intra-arterial, intraocular, intraorbital, intradermal, subcutaneous, oral (e.g., inhalation), transdermal (topical-particularly to the ears, nose, eyes, or skin), transmucosal (e.g., oral) nasal, rectal, intracerebral, intravaginal, sublingual, submucosal, and transdermal administration.

Administration can be via any route known to be effective by a physician of ordinary skill. Parenteral administration, i.e., not through the alimentary canal, can be performed by subcutaneous, intramuscular, intra-peritoneal, intratumoral, intradermal, intracapsular, intra-adipose, or intravenous injection of a dosage form into the body by means of a sterile syringe, optionally a pen-like syringe, or some other mechanical device such as an infusion pump. A further option is a composition that can be a powder or a liquid for the administration in the form of a nasal or pulmonary spray. As a still further option, the administration can be transdermally, e.g., from a patch. Compositions suitable for oral, buccal, rectal, or vaginal administration can also be provided. In a preferred embodiment, administration of the polyamine compound of present invention is via an intravenous injection, e.g. an intravenous bolus injection or by gradual perfusion over time.

The polyamine compound and the pharmaceutical composition of present invention can also be administered by a small bolus injection followed by a continuous infusion. One protocol for treatment with spermidine or a spermidine analog is as follows: (i) initial bolus injection over a period of 1-2 minutes; (ii) high level infusion for 1 hour; (2) low level maintenance infusion for 2-3 hours.

The whole of the dose of spermidine required to achieve a protective effect could also be administered as one or more bolus injections e.g. ranging between 1-100 percent of the estimated required 24 h dose, or administered with a 50 cc syringe at a rate of 2 ml per hour.

The polyamine compound and the pharmaceutical composition of present invention can also be administered by a small bolus injection followed by a continuous infusion. One protocol for treatment with spermidine or a spermidine analog is as follows: (i) initial bolus injection over a period of 1-2 minutes; (ii) high level infusion for 1 hour; (2) low level maintenance infusion for 2-3 hours.

The whole of the dose of spermidine required to achieve a protective effect could also be administered as one or more bolus injections, e.g. administered with a 50 cc syringe at a rate of 2 ml over 1 hour.

In one embodiment, a pharmaceutical composition of the invention is delivered by a controlled release system. For example, the pharmaceutical composition can be administered using intravenous infusion, an implantable osmotic pump, a transdermal patch, liposomes, or other modes of administration. In one embodiment, a pump can be used (See e.g., Langer (1990) Science 249, 1527-1533; Sefton (1987) CRC Crit. Ref. Biomed. Eng. 14, 201; Buchwald et al. 1(980) Surgery 88, 507; Saudek et al., (1989) N. Engl. J. Med. 321, 574). In another embodiment, the compound can be delivered in a vesicle, in particular a liposome (See e.g., Langer (1990) Science 249, 1527-1533; Treat et al., (1989) in Liposomes in the Therapy of Infectious Disease and Cancer, Lopez-Berestein and Fidler (eds.), Liss, New York, pp. 353-365; Lopez-Berestein, ibid., pp. 317-27; International Patent Publication No. WO 91/04014; U.S. Pat. No. 4,704,355). In another embodiment, polymeric materials can be used (See e.g., Medical Applications of Controlled Release, Langer and Wise (eds.), CRC Press: Boca Raton, Fla., 1974; Controlled Drug Bioavailability, Drug Product Design and Performance, Smolen and Ball (eds.), Wiley: New York (1984); Ranger and Peppas, (1953) J. Macromol. Sci. Rev. Macromol. Chem. 23, 61; Levy et al. (1985), Science 228, 190; During et al. (1989) Ann. Neurol. 25, 351; Howard et al. (1989) J. Neurosurg. 71, 105).

In yet another embodiment, a controlled release system can be placed in proximity of the target. For example, a micropump can deliver controlled doses directly into bone or adipose tissue, thereby requiring only a fraction of the systemic dose (See e.g., Goodson (1984), Medical Applications of Controlled Release 2, 115-138). In another example, a pharmaceutical composition of the invention can be formulated with a hydrogel (See, e.g., U.S. Pat. Nos. 5,702,717; 6,117,949; 6,201,072).

In one embodiment, it may be desirable to administer the pharmaceutical composition of the invention locally, i.e., to the area in need of treatment. Local administration can be achieved, for example, by local infusion during surgery, topical application (e.g., in conjunction with a wound dressing after surgery), injection, catheter, suppository, or implant. An implant can be of a porous, non-porous, or gelatinous material, including membranes, such as sialastic membranes, or fibers.

In certain embodiments, it may be desirable to introduce the polyamine compound into the central nervous system by any suitable route, including intraventricular, intrathecal, and epidural injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant.

In one embodiment, the invention provides for the treatment of a patient using implanted cells that have been regenerated or stimulated to proliferate in vitro or in vivo prior to reimplantation or transplantation into a recipient. Conditioning of the cells ex vivo can be achieved simply by growing the cells or tissue to be transplanted in a medium that has been supplemented with a growth-promoting amount of the combinations and is otherwise appropriate for culturing of those cells. The cells can, after an appropriate conditioning period, then be implanted either directly into the patient or can be encapsulated using established cell encapsulation technology, and then implanted.

The skilled artisan can appreciate the specific advantages and disadvantages to be considered in choosing a mode of administration. Multiple modes of administration are encompassed by the invention. For example, a polyamine compound of the invention can be administered by subcutaneous injection, whereas another therapeutic agent can be administered by intravenous infusion. Moreover, administration of one or more species of polyamine compounds, with or without other therapeutic agents, can occur simultaneously (i.e., co-administration) or sequentially. In another embodiment, the periods of administration of a polyamine compound, with or without other therapeutic agents can overlap. For example a polyamine compound can be administered for 7 days and another therapeutic agent can be introduced beginning on the fifth day of polyamine compound treatment. Treatment with the other therapeutic agent can continue beyond the 7-day polyamine compound treatment.

A pharmaceutical composition of a polyamine compound can be administered before, during, and/or after the administration of one or more therapeutic agents. In one embodiment, polyamine compound can first be administered to stimulate the expression of insulin, which increases sensitivity to subsequent challenge with a therapeutic agent. In another embodiment, polyamine compound can be administered after administration of a therapeutic agent. In yet another embodiment, there can be a period of overlap between the administration of the polyamine compound and the administration of one or more therapeutic agents.

A pharmaceutical composition of the invention can be administered in the morning, afternoon, evening, or diurnally. In one embodiment, the pharmaceutical composition is administered at particular phases of the circadian rhythm. In a specific embodiment, the pharmaceutical composition is administered in the morning. In another specific embodiment, the pharmaceutical composition is administered at an artificially induced circadian state.

The present pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (See e.g., U.S. Pat. No. 5,698,155).

Pharmaceutical compositions adapted for parenteral administration include, but are not limited to, aqueous and non-aqueous sterile injectable solutions or suspensions, which can contain antioxidants, buffers, bacteriostats and solutes. Other components that can be present in such pharmaceutical compositions include water, alcohols, polyols, glycerine and vegetable oils, for example. Compositions adapted for parenteral administration can be presented in unit-dose or multi-dose containers (e.g., sealed ampoules and vials), and can be stored in a freeze-dried (i.e., lyophilized) condition requiring the addition of a sterile liquid carrier (e.g., sterile saline solution for injections) immediately prior to use. Extemporaneous injection solutions and suspensions can be prepared from sterile powders, granules and tablets.

Pharmaceutical compositions adapted for transdermal administration can be provided as discrete patches intended to remain in intimate contact with the epidermis for a prolonged period of time. Pharmaceutical compositions adapted for topical administration can be provided as, for example, ointments, creams, suspensions, lotions, powders, solutions, pastes, gels, sprays, aerosols or oils. A topical ointment or cream is preferably used for topical administration to the skin, mouth, eye or other external tissues. When formulated in an ointment, the active ingredient can be employed with either a paraffinic or a water-miscible ointment base. Alternatively, the active ingredient can be formulated in a cream with an oil-in-water base or a water-in-oil base.

Pharmaceutical compositions adapted for topical administration to the eye include, for example, eye drops or injectable pharmaceutical compositions. In these pharmaceutical compositions, the active ingredient can be dissolved or suspended in a suitable carrier, which includes, for example, an aqueous solvent with or without carboxymethylcellulose. Pharmaceutical compositions adapted for topical administration in the mouth include, for example, lozenges, pastilles and mouthwashes. Pharmaceutical compositions adapted for nasal administration can comprise solid carriers such as powders (preferably having a particle size in the range of 20 to 500 microns). Powders can be administered in the manner in which snuff is taken, i.e., by rapid inhalation through the nose from a container of powder held close to the nose. Alternatively, pharmaceutical compositions adopted for nasal administration can comprise liquid carriers such as, for example, nasal sprays or nasal drops. These pharmaceutical compositions can comprise aqueous or oil solutions of a polyamine compound. Compositions for administration by inhalation can be supplied in specially adapted devices including, but not limited to, pressurized aerosols, nebulizers or insufflators, which can be constructed so as to provide predetermined dosages of the polyamine compound.

Typically, pharmaceutical compositions for injection or intravenous administration are solutions in sterile aqueous buffers. Where necessary, the composition can also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically sealed container such as an ampoule or sachet indicating the quantity of active agent.

Where the composition is to be administered by infusion, it can be dispensed with an infusion bottle, bag, or other acceptable container, containing sterile pharmaceutical grade water, saline, or other acceptable diluents. Where the composition is administered by injection, an ampoule of sterile water for injection or saline can be provided so that the ingredients may be mixed prior to administration.

For a patient who cannot orally ingest a nutrient, it is essential to supply all nutrients such as an amino acid, a saccharide and an electrolyte through a vein. This way is called the total parenteral nutrition therapy, (TPN therapy) which can be provided by a TPN solution. Such TPN solutions are particularly suitable for critically ill patients for a therapy in the Intensive Care Unit. As a TPN solution employed in the TPN therapy, there has been known (1) a TPN solution containing a saccharide, an amino acid, a fat and an electrolyte (Japanese Unexamined Patent Publications No. 186822/1989, WO08503002 and EP-A-0 399 341), (2) an emulsion for injection comprising an amino acid and a fat (Japanese Unexamined Patent Publication No. 74637/1986), (3) a TPN solution comprising two separate infusions, one of which contains glucose and an electrolyte and the other of which contains an amino acid (Japanese Unexamined Patent Publications No. 52455/1982 and No. 103823/1986) and the like. In the TPN therapy, an infusion containing a high concentration of saccharide is usually administered to a patient.

As indicated the high nutritional content of such TPN solutions may lead to hyperglycemia and has been found to have a detrimental effect on the repair processes in critically ill patients by inhibition the autophagy process, which contributes to the removal of damaged organelles.

Accordingly, a further aspect of the present invention relates to a TPN solution combined with a polyamine compound of the present invention. This combined composition is used to improve the condition of a critically ill patient or to reduce or treat multiple organ dysfunction syndrome in a critically ill patient.

Compositions for parenteral nutrition, in particular for intravenous administration are isotonic or hypertonic solutions (e.g. prepared by NaCl and/or dextrose or lactated Ringers) further comprising a saccharide such as glucose in a range between 10 and 20% (w/v) to obtain a high nutritional content, and further comprising lipids and/or amino acids and/or vitamines.

Compositions for parenteral administration comprise further to polyamine compound of the present invention a saccharide such as glucose. Final glucose concentrations in a composition for administration are typically in the range from 10 to 20% (w/v) e.g. 12.5 or 16%.

Compositions for parenteral administration typically further comprise saturated, mono-unsaturated and essential poly-unsaturated fatty acids such as refined olive oil and/or soybean oil. Final lipid concentrations in a composition for administration are typically in the range of 2 to 6% (w/v) e.g. 4%.

Compositions for parenteral administration typically further comprise one or more amino acids. Final amino acid concentrations are typically in the range from 2 to 6% (w/v) e.g. 4%.

Compositions for parenteral administration optionally further comprise trace elements such as one or more of Fe, Zn, Cu, Mn, F, Co, I, Se, Mo, Cr e.g. under the form of respectively the following salts ferrous gluconate, copper gluconate, manganese gluconate, zinc gluconate, sodium fluoride, cobalt II gluconate, sodium iodide, sodium selenite, ammonium molybdate and chromic chloride.

Compositions for parenteral administration optionally further comprise one or more vitamins such as Vitamin A (Retinol), Vitamin D3, Vitamin E (α tocopherol), Vitamin C, Vitamin B1 (thiamine), Vitamin B2 (riboflavin), Vitamin B6 (pyridoxine), Vitamin B12, Folic Acid, Pantothenic acid, Biotin, and Vitamin PP (niacin), e.g. under the form of Retinol palmitate, Colecalciferol, DL-α-tocopherol, Ascorbic acid, Cocarboxylase tetrahydrate, Riboflavin dihydrated sodium phosphate, Pyridoxine hydrochloride, Cyanocobalamin, Folic acid, Dexpanthenol, D-Biotin and Nicotinamide.

Compositions for parenteral administration prior to administration can be isotonic solutions, or more particularly hypertonic solutions e.g. solutions with osmolarity between 1000 and 1500, or between 1200 and 1500 mOsm/liter, e.g. 1250 or 1500 mOsm/liter.

Compositions for parenteral administration can be provided as one solution comprising all constituents or as a kit of parts wherein different consituents are provided separately (saccharide, lipids, amino acids) and wherein the polyamine is dissolved in one of the constituents or is provided seperately. One or more of the different constituents may be provided in a dried form, which is redissolved prior to use.

The compositions for parenteral nutrition in accordance with the present invention further comprise a polyamine such as spermine, spermidine or putrescine in a concentration between 0.05, 0.1, 0.2 or 0.5 to 1, 2, 3 or 4% (w/v).

The compostions for intravenous administration are typically packed in plastic bags with spike ports for delivery by intravenous drips.

In a specific embodiment, the present compositions contain spermine or spermidine. For patients which do not rely on parenteral food pharmaceutical compositions herein described can be provided in the form of oral tablets, capsules, elixirs, syrups and the like.

Compositions for oral administration might require an enteric coating to protect the composition(s) from degradation within the gastrointestinal tract. In another example, the composition(s) can be administered in a liposomal formulation to shield the polyamine compound disclosed herein from degradative enzymes, facilitate the molecule's transport in the circulatory system, and affect delivery of the molecule across cell membranes to intracellular sites.

A polyamine compound intended for oral administration can be coated with or admixed with a material (e.g., glyceryl monostearate or glyceryl distearate) that delays disintegration or affects absorption of the polyamine compound in the gastrointestinal tract. Thus, for example, the sustained release of a polyamine compound can be achieved over many hours and, if necessary, the polyamine compound can be protected from being degraded within the gastrointestinal tract. Taking advantage of the various pH and enzymatic conditions along the gastrointestinal tract, pharmaceutical compositions for oral administration can be formulated to facilitate release of a polyamine compound at a particular gastrointestinal location.

Selectively permeable membranes surrounding an osmotically active driving compound are also suitable for orally administered compositions. Fluid from the environment surrounding the capsule is imbibed by the driving compound, which swells to displace the polyamine compound through an aperture, can provide an essentially zero order delivery profile instead of the spiked profiles of immediate release formulations. A time delay material such as, but not limited to, glycerol monostearate or glycerol stearate can also be used.

Suitable pharmaceutical carriers also include starch, glucose, lactose, sucrose, gelatin, saline, gum acacia, talc, keratin, urea, malt, rice, flour, chalk, silica gel, sodium stearate, glycerol monostearate, talc, sodium chloride, dried skim milk, glycerol, propylene, glycol, water, and ethanol. If desired, the carrier, can also contain minor amounts of wetting or emulsifying agents, or pH buffering agents. In addition, auxiliary, stabilizing, thickening, lubricating, and coloring agents may be used. The composition can be formulated as a suppository, with traditional binders and carriers such as triglycerides.

For oral administration in the form of a tablet or capsule, the active drug component can be combined with an oral, non-toxic, pharmaceutically acceptable, inert carrier such as, but not limited to, lactose, starch, sucrose, glucose, methyl cellulose, magnesium stearate, dicalcium phosphate, calcium sulfate, mannitol, and sorbitol. For oral administration in liquid form, the oral drug components can be combined with any oral, non-toxic, pharmaceutically acceptable carrier such as, but not limited to, ethanol, glycerol, and water. Moreover, suitable binders, lubricants, disintegrating agents and coloring agents can also be incorporated into the mixture. Suitable binders include, but are not limited to, starch, gelatin, natural sugars (e.g., glucose, beta-lactose), corn sweeteners, natural and synthetic gums (e.g., acacia, tragacanth, sodium alginate), carboxymethylcellulose, polyethylene glycol, and waxes. Lubricants useful for an orally administered drug, include, but are not limited to, sodium oleate, sodium stearate, magnesium stearate, sodium benzoate, sodium acetate, and sodium chloride. Disintegrators include, but are not limited to, starch, methyl cellulose, agar, bentonite, and xanthan gum.

Pharmaceutical compositions adapted for oral administration can be provided, for example, as capsules or tablets; as powders or granules; as solutions, syrups or suspensions (in aqueous or non-aqueous liquids); as edible foams or whips; or as emulsions. For oral administration in the form of a tablet or capsule, the active drug component can be combined with an oral, non-toxic, pharmaceutically acceptable, inert carrier such as, but not limited to, lactose, starch, sucrose, glucose, methyl cellulose, magnesium stearate, dicalcium phosphate, magnesium carbonate, stearic acid or salts thereof, calcium sulfate, mannitol, and sorbitol. For oral administration in the form of a soft gelatin capsule, the active drug component can be combined with an oral, non-toxic, pharmaceutically acceptable, inert carrier such as, but not limited to, vegetable oils, waxes, fats, semi-solid, and liquid polyols. For oral administration in liquid form, the oral drug components can be combined with any oral, non-toxic, pharmaceutically acceptable carrier such as, but not limited to, ethanol, glycerol, polyols, and water. Moreover, suitable binders, lubricants, disintegrating agents and coloring agents can also be incorporated into the mixture. Suitable binders include, but are not limited to, starch, gelatin, natural sugars (e.g. glucose, beta-lactose), corn sweeteners, natural and synthetic gums (e.g., acacia, tragacanth, sodium alginate), carboxymethylcellulose, polyethylene glycol, and waxes. Lubricants useful for an orally administered drug, include, but are not limited to, sodium oleate, sodium stearate, magnesium stearate, sodium benzoate, sodium acetate, and sodium chloride. Disintegrators include, but are not limited to, starch, methyl cellulose, agar, bentonite, and xanthan gum.

Orally administered compositions may contain one or more agents, for example, sweetening agents such as, but not limited to, fructose, aspartame and saccharin. Orally administered compositions may also contain flavoring agents such as, but not limited to, peppermint, oil of wintergreen, and cherry. Orally administered compositions may also contain coloring agents and/or preserving agents.

The polyamine compounds of present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. A variety of cationic lipids can be used in accordance with the invention including, but not limited to, N-(1(2,3-dioleyloxy)propyl)-N,N,N-trimethylammonium chloride (“DOTMA”) and diolesylphosphotidylethanolamine (“DOPE”). Such compositions suit the mode of administration.

The polyamine compounds of present invention can also be delivered by the use of monoclonal antibodies as individual carriers to which the compounds can be coupled. The compounds can also be coupled with soluble polymers as targetable drug carriers. Such polymers can include polyvinylpyrrolidone, pyran copolymer, polyhydroxypropylmethacrylamide-phenol, polyhydroxyethylaspartamide-phenol, or polyethyleneoxide-polylysine substituted with palmitoyl residues. Furthermore, the polyamine compounds can be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

Pharmaceutical compositions adapted for rectal administration can be provided as suppositories or enemas. Pharmaceutical compositions adapted for vaginal administration can be provided, for example, as pessaries, tampons, creams, gels, pastes, foams or spray formulations.

Suppositories generally contain active ingredients in the range of 0.5% to 10% by weight. Oral formulations preferably contain 10% to 95% active ingredient by weight. In a preferred embodiment, the composition is formulated in accordance with routine procedures as a pharmaceutical composition adapted for intratumoral injection, implantation, subcutaneous injection, or intravenous administration to humans.

Conveniently, the blood spermidine level is kept within the ranges mentioned in connection with the present invention for as long a period of time as the patient is critically ill. Hence, as a general rule, the blood spermidine level is kept within the ranges mentioned in connection with the present invention as long as the patient is critically ill. Consequently, the blood spermidine level is usually kept within the ranges mentioned in connection with the present invention for a period of time of more than about 8 hours, preferably more than about 24 hours, even more preferred more than about 2 days, especially more than about 4 days, and even more than about 7 days. In certain cases, it may even be preferred that the blood spermidine level is kept within the ranges mentioned in connection with the present invention after the patient (previously) considered as being critically ill has been transferred from the Intensive Care Unit to another part of the hospital or even after the patient has left the hospital.

A critically ill patient, optionally entering an ICU, may be fed continuously, on admission with mainly intravenous glucose (for example, about 200 g to about 300 g per 24 hours) and from the next day onward with a standardised feeding schedule aiming for a caloric content up to between about 10 and about 40, preferably between about 20 and about 30, non-protein Calories/kg/24 hours and a balanced composition (for example, between about 0.05 and about 0.4, preferably between about 0.13 and about 0.26 g nitrogen/kg/24 hours and between about 20% and about 40% of non-protein Calories as lipids) of either total parenteral, combined parenteral/enteral or full enteral feeding, the latter mode attempted as early as possible. Other concomitant ICU therapy can be left to the discretion of attending physicians.

Alternatively, the following procedure can be used or it is possible to use a combination or variant of these procedures, as the physician considers advantageous for the patient:

A critically ill patient may be fed, on the admission day, using, for example, a 20% glucose infusion and from day 2 onward by using a standardised feeding schedule consisting of normal caloric intake (for example, about 25-35 Calories/kgBW/24 h) and balanced composition (for example, about 20%-40% of the non-protein Calories as lipids and about 1-2 g/kgBW/24 h protein and about 0.01-100 mg/kg BW/24 h spermidine) of either total parenteral, combined parenteral/enteral or full enteral feeding, the route of administration of feeding depending on assessment of feasibility of early enteral feeding by the attending physician. All other treatments, including feeding regimens, were according to standing orders currently applied within the ICU.

The polyamine compound and optionally another therapeutic agent are administered at an effective dose. The dosing and regimen most appropriate for patient treatment will vary with the disease or condition to be treated, and in accordance with the patient's weight and with other parameters.

An effective dosage and treatment protocol can be determined by conventional means, comprising the steps of starting with a low dose in laboratory animals, increasing the dosage while monitoring the effects (e.g., histology, disease activity scores), and systematically varying the dosage regimen. Several factors may be taken into consideration by a clinician when determining an optimal dosage for a given patient. Additional factors include, but are not limited to, the size of the patient, the age of the patient, the general condition of the patient, the particular disease being treated, the severity of the disease, the presence of other drugs in the patient, and the in vivo activity of the polyamine compound.

A typical effective human dose of a polyamine compound would be from about 10 μg/kg body weight/day to about 100 mg/kg/day, preferably from about 50 μg/kg/day to about 50 mg/kg/day, and most preferably about 100 μg/kg/day to 20 mg/kg/day. As analogs of the polyamine compound disclosed herein can be 2 to 100 times more potent than naturally occurring counterparts, a typical effective dose of such an analog can be lower, for example, from about 100 μg/kg body weight/day to 1 mg/kg/day, preferably 10 μg/kg/day to 900 μg/kg/day, and even more preferably 20 μg/kg/day to 250 μg/kg/day.

In another embodiment, the effective dose of a polyamine compound of present is less than 10 μg/kg/day. In yet another embodiment the effective dose of a polyamine compound of present is greater than 10 mg/kg/day.

The specific dosage for a particular patient, of course, has to be adjusted to the degree of response, the route of administration, the patient's weight, and the patient's general condition, and is finally dependent upon the judgment of the treating physician. Especially the highly critical condition of ICU patients requires a specific dosage and dosage regime.

It is understandable that the ideal dosage per serving to have the health effect will have to vary according the body weight of the subject who consumes the oral ingestible dosage form which comprises the polyamine compound of present invention. A beneficial effect can be obtained in a subject with about 50 kg body weight by an orally ingestible dosage form comprising between 0.5 mg and 5 gram, preferably 15 mg to 2 gram, more preferably between 25 mg and 1.5 gram, more preferably between 50 mg and 750 mg of the polyamine compound of present invention per administration (as demonstrated in Table 1).

TABLE 1
Possible amount of the polyamine compound active ingredient of present
invention per serving by a subject (BW: body weight).

| | BW/kg | | | | | | | | | |
|---|---|---|---|---|---|---|---|---|---|---|
| Dose | 50 | 60 | 70 | 80 | 90 | 100 | | 120 | | |
| mg/kg | mg | mg | mg | mg | mg | mg | 110 mg | mg | 130 mg | 140 mg |
| 0.1 | 5 | 6 | 7 | 8 | 9 | 10 | 11 | 12 | 13 | 14 |
| 0.2 | 10 | 12 | 14 | 16 | 18 | 20 | 22 | 24 | 26 | 28 |
| 0.3 | 15 | 18 | 21 | 24 | 27 | 30 | 33 | 36 | 39 | 42 |
| 0.4 | 20 | 24 | 28 | 32 | 36 | 40 | 44 | 48 | 52 | 56 |
| 0.5 | 25 | 30 | 35 | 40 | 45 | 50 | 55 | 60 | 65 | 70 |
| 1 | 50 | 60 | 70 | 80 | 90 | 100 | 110 | 120 | 130 | 140 |
| 5 | 250 | 300 | 350 | 400 | 450 | 500 | 550 | 600 | 650 | 700 |
| 10 | 500 | 600 | 700 | 800 | 900 | 1000 | 1100 | 1200 | 1300 | 1400 |
| 15 | 750 | 900 | 1050 | 1200 | 1350 | 1500 | 1650 | 1800 | 1950 | 2100 |
| 20 | 1000 | 1200 | 1400 | 1600 | 1800 | 2000 | 2200 | 2400 | 2600 | 2800 |
| 25 | 1250 | 1500 | 1750 | 2000 | 2250 | 2500 | 2750 | 3000 | 3250 | 3500 |
| 30 | 1500 | 1800 | 2100 | 2400 | 2700 | 3000 | 3300 | 3600 | 3900 | 4200 |
| 35 | 1750 | 2100 | 2450 | 2800 | 3150 | 3500 | 3850 | 4200 | 4550 | 4900 |
| 40 | 2000 | 2400 | 2800 | 3200 | 3600 | 4000 | 4400 | 4800 | 5200 | 5600 |
| 45 | 2250 | 2700 | 3150 | 3600 | 4050 | 4500 | 4950 | 5400 | 5850 | 6300 |
| 50 | 2500 | 3000 | 3500 | 4000 | 4500 | 5000 | 5500 | 6000 | 6500 | 7000 |

A beneficial effect can also be obtained in a subject with about 50 kg body weight as part of a TPN therapy comprising between 0.5 mg and 2.5 gram, preferably 15 mg to 2 gram, more preferably between 25 mg and 1.5 gram, more preferably between 50 mg and 750 mg of the polyamine compound of present invention per administration.

Also contemplated are methods of prevention or treatment involving combination therapies comprising administering an effective amount of the polyamine compound molecule of present invention, eventually in combination with another therapeutic agent or agents. The other therapeutic agent or agent can be, for example, an anti-osteoporosis agent, a steroid hormones, a non-steroid hormone, growth factor, a selective estrogen receptor modulator, an insulin-releasing agent, an inhibitor of glucagon secretion, a glucagon antagonists, a circadian rhythm regulator, a growth hormone secretagogue, an agent that increases IGF-1 levels, an immunotherapeutic agent, a cytokine, a protease inhibitor, a vitronectin receptor antagonist, a bisphosphonate compound, a kinase inhibitor, an integrin receptor or antagonist thereof, an anti-obesity agent, a lipid-metabolism improving agent, a neuropeptide Y blocker, a kainate/AMPA receptor antagonist, a β-adrenergic receptor agonist, a compound that reduces caloric intake, an anti-diabetes agent, or a dietary nutrient. Examples of therapeutic agents include, but are not limited to:
anti-osteoporosis agent, such as alendronate sodium, calcium L-threonate (e.g., C8H14O10Ca) clodronate, etidronate, gallium nitrate, mithramycin, norethindrone acetate (e.g., that which is commercially available as ACTIVELLA) osteoprotegerin pamidronate and risedronate sodium,
steroid hormones, such as androgen (e.g., androstenedione, testosterone, dehydroepiandrosterone, dihydrotestosterone, 7-alpha-methyl-19-nortestosterone, alpha-methyl-19-nortestosterone acetate, methandroil, oxymetholone, methanedione, oxymesterone, nordrolone phenylpropionate, noretbandrolone), glucocorticoids, estrogenic hormones (e.g., that which is commercially available as PREMARIN) and progestin,
non-steroid hormones such as calcitonin, calcitriol growth hormone (e.g., osteoclast-activating factor), melatonin, parathyroid hormone prostaglandin, thyroid hormone.
growth factors such as epidermal growth factor, fibroblast growth factor, insulin-like growth factor 1, insulin-like growth factor 2, platelet-derived growth factor, vascular endothelial growth factor,
selective estrogen receptor modulator such as, BE-25327, CP-336156, clometherone, delmadinone, droloxifene, idoxifene, nafoxidine, nitromifene, ormeloxifene, raloxifene (e.g., that which is commercially available as EVISTA), tamoxifen, toremifene, trioxifene, [2-(4-hydroxyphenyl)-6-hydroxynaphthalen-1-yl][4-[2-(1-piperidinyl)-ethoxy]phenyl]-methane,
Insulin-releasing agent such as GLP-1, nateglinide, repaglinide (e.g., that which is commercially available as PRANDIN), sulfonylurea (e.g., glyburide, glipizide, glimepiride),
vasopressin,
inhibitor of glucagon secretion, such as somatostatin, glucagon antagonists, substituted glucagons having an alanine residue at position 1, 2, 3-5, 9-11, 21, or 29, des-His1-Ala2 glucagons, des-His1-[Ala2,11-Glu21]glucagon,
circadian rhythm regulators such as alkylene dioxybenzene agonist, melatonin, neuropeptide Y, tachykinin agonist, visible light therapy, growth hormone secretagogue, cycloalkano[b]thien-4-ylurea, GHRP-1, GHRP-6, growth hormone releasing factor, hexarelin, thiourea, B-HT920, benzo-fused lactams (e.g., N-biphenyl-3-amido substituted benzolactams), benzo-fused macrocycles (e.g., 2-substituted piperidines, 2-substituted pyrrolidines, 2-substituted hexahydro-1H-azepines, di-substituted piperidines, di-substituted pyrrolidines, di-substituted hexahydro-1H-azepines, tri-substituted piperidines, tri-substituted pyrrolidines, tri-substituted hexahydro-1H-azepines, L-pyroglutamyl-pyridylalanyl-L-prolinamides),
agents that increase IGF-1 levels such as L-acetylcamitine, L-isovalerylcamitine, L-propionylcarnitine,
immunotherapeutic agents such as antibodies and immunomodulators,
cytokine such as endothelial monocyte activating protein, granulocyte colony stimulating factor, such as interferon (e.g., IFN-γ), interleukin (e.g., IL-6),
lymphokine such as, lymphotoxin-α, lymphotoxin-β,
tumor necrosis factor, tumor necrosis-factor-like cytokine, macrophage inflammatory protein, monocyte colony stimulating factor, 4-1BBL, CD27 ligand, CD30 ligand, CD40 ligand, CD137 ligand, Fas ligand, OX40 ligand,
protease inhibitors such as cysteine protease inhibitor (e.g., vinyl sulfone, peptidylfluoromethyl ketone, cystatin C, cystatin D, E-64), DPP IV antagonist, DPP IV inhibitor (e.g., N-(substituted glycyl)-2-cyanopyrrolidines, N-Ala-Pro-Onitrobenzyl-hydroxylamine, and ε-(4-nitro)benzoxycarbonyl-Lys-Pro), serine-protease inhibitor (e,g., azapeptide, BMS232632, antipain, leupeptin),
vitronectin receptor antagonist, anti-vitronectin receptor antibody (e.g., 23C6), cyclo-S,S-N α-acetyl-cysteinyl-N alpha-methyl-argininyl-glycyl-aspartyl penicillamine, RGD-containing peptide (e.g., echistatin), bisphosphonate compound, alendronate (e.g., that which is commercially available as FOSAMAX), aminoalkyl bisphosphonate, (e.g., alendronate, pamidronate (3-amino-1-hydroxypropylidene)bisphosphonic acid disodium salt, pamidronic acid, risedronate (1-hydroxy-2-(3-pyridinyl)ethylidene)bisphosphonate, YM 175 [(cycloheptylamino)methylene-bisphosphonic acid], piridronate, aminohexane-bisphosphonate, tiludronate, BM-210955, CGP-42446, EB-1053), risedronate (commercially available as ACTONEL),
kinase inhibitors, such as Rho-kinase inhibitor (e.g., (+)-trans-4-(1-aminoethyl)-1-(4-pyridylcarbamoyl)cyclohexane, trans-N-(1H-pyrrolo[2,3-b]pyridin-4-yl)-4-guanidino-methylcyclohexanecarbox amide, 1-(5-isoquinolinesulfonyl)homopiperazine, 1-(5-isoquinolinesulfonyl)-2-methylpiperazine),
integrin receptor, α subunit (e.g., subtype 1-9, D, M, L, X, V, IIb, IELb), β subunit (e.g., subtype 1-8),
integrin receptor antagonists, ethyl 3(S)-(2,3-dihydro-benzofuran-6-yl)-3-{2-oxo-3-[3-(5,6,7,8-tetrahydro-[1,8]naphthyridin-2-yl)-propyl]-tetrahydro-pyrimidin-1-yl}-propionate; ethyl 3(S)-(3-fluorophenyl)-3-(2-oxo-3(S or R)-[3-(5,6,7,8-tetrahydro-[1,8]naphthyridin-2-yl)-propyl]-piperidin-1-yl)-propionate; ethyl 3(S)-(3-fluorophenyl)-3-(2-oxo-3(R) or S)-[3-(5,6,7,8-tetrahydro-[1,8]naphthyridin-2-yl)-propyl]-piperidin-1-yl)-propio nate; 3(S)-(2,3-dihydro-benzofuran-6-yl)-3-{2-oxo-3-[3-(5,6,7,8-tetrahydro-[1,8]n aphthyridin-2-yl-propyl]-tetrahydro-pyrimidin-1-yl}-propionic acid; 3(S)-(3-fluorophenyl)-3-(2-oxo-3(R) or R)-[3-(5,6,7,8-tetrahydro-[1,8]naphthyridin-2-yl)-propyl]-piperidin-1-yl)-propionic acid; 3(S)-(3-fluorophenyl)-3-(2-oxo-3(S or S)-[3-(5,6,7,8-tetrahydro-[1,8]naphthyridin-2-yl)-propyl]-piperidin-1-yl)-propionic acid,
anti-obesity agents such as benzphetamine (commercially available as DIDREX), benzylisopropylamine (commercially available as IONAMIN), bupropion, dexfenfluramine (commercially available as REDUX), dextroamphetamine (commercially available as DEXEDRINE), diethylpropion (commercially available as TENUATE), dimethylphenethylamine (commercially available as ADIPEX or DESOXYN), evodamine, fenfluramine (commercially available as PONDIMIN), fluoxetine, mazindol (commercially available as SANOREX or MAZANOR), methamphetamine, naltrexone, orlistat (commercially available as XENICAL), phendimetrazine (commercially available as BONTRIL or PLEGINE), phentermine (commercially available as FASTIN), sibutramine (commercially available as MERIDIA), a lipid-metabolism improving agent, capsaicin, an neuropeptide Y blocker, NGD-95-1, kainate/AMPA receptor antagonist, β-adrenergic receptor agonist, compound that reduces caloric intake, fat substitute (e.g., that which is commercially available as OLESTRA), sugar substitute (e.g., that which is commercially available as ASPARTAME), anti-diabetes agent, insulin glargine (commercially available as LANTUS), pioglitazone (commercially available as ACTOS), rosiglitazone maleate (commercially available as AVANDIA),
dietary nutrients such as sugar; dietary fatty acid, triglyceride, oligosaccharides (e.g., fructo-oligosaccharides, raffinose, galacto-oligosaccharides, xylo-oligosaccharides, beet sugar and soybean oligosaccharides), protein, vitamin (e.g., vitamin D), mineral (e.g., calcium, magnesium, phosphorus and iron),

The other therapeutic agents can be made and used at doses as disclosed previously. For example, an anti-osteoporosis agent (see e.g., U.S. Pat. Nos. 2,565,115 and 2,720,483), a non-steroid hormone (see, e.g., U.S. Pat. Nos. 6,121,253; 3,927,197; 6,124,314), a glucagon antagonists (see, e.g., U.S. Pat. No. 5,510,459), a growth hormone secretagogue (see, e.g., U.S. Pat. Nos. 3,239,345; 4,036,979; 4,411,890; 5,206,235; 5,283,241; 5,284,841; 5,310,737; 5,317,017; 5,374,721; 5,430,144; 5,434,261; 5,438,136; 5,494,919; 5,494,920; and 5,492,916; European Patent Nos. 144,230 and 513,974; International Patent Publication Nos. WO 89/07110; WO 89/07111; WO 93/04081; WO 94/07486; WO 94/08583; WO 94/11012; WO 94/13696; WO 94/19367; WO 95/03289; WO 95/03290; WO 95/09633; WO 95/11029; WO 95/12598; WO 95/13069; WO 95/14666; WO 95/16675; WO 95/16692; WO 95/17422; WO 95/17423; WO 95/34311; and WO 96/02530), an agent that increase IGF-1 levels (see, e.g., U.S. Pat. No. 6,166,077), a cytokine (see, e.g., U.S. Pat. No. 4,921,697), a vitronectin receptor antagonist (see e.g., U.S. Pat. No. 6,239,138 and Horton et al., (1991) Exp. Cell Res. 195, 368), a bisphosphonate compound (see e.g., U.S. Pat. No. 5,409,911), a kinase inhibitor (U.S. Pat. No. 6,218,410), and an integrin receptor or antagonist thereof (see, e.g., U.S. Pat. No. 6,211,191).

### EXAMPLES

The terminology used herein is for the purpose of describing particular embodiments only, and is not intended to limit the scope of the present invention which will be limited only by the appending claims. This invention is not limited to the particular methodology, protocols, delivery forms and reagents described as these may vary.

### Example 1

### Animal Model for Critical Illness

Our research group previously developed an animal model of critical illness that has shown to mimic the dynamic endocrine, immunological and metabolic changes characteristic of human critical illness. In this animal model we investigated the effect of critical illness on spermidine levels and the effects of spermidine-administration during critical illness on survival, organ function (clinical, biochemical, and cyto/histopathological effects), and on metabolic, inflammatory/immunological and cellular pathways. Animals were treated according to the “Principals of Laboratory Animal Care” formulated by the U.S. National Society for Medical Research and the “Guide for the Care and Use of Laboratory Animals” prepared by the National Institutes of Health. The protocol was approved by the K.U. Leuven Ethical Review Board for Animal Research. Adult male New Zealand White rabbits, weighing approximately 3 kg, were purchased from a local rabbitry, were housed individually with free access to water, hay and regular rabbit chow, and were exposed to artificial light for 14 h per day. This animal model of prolonged critical illness mimics the human condition (Weekers et al. (2003) Endocrinol 144, 5329-5338). Indeed, the critically ill animals undergo the same metabolic, immunological and endocrine disturbances and development of organ failure and muscle wasting as the human counterpart. In this animal model, we previously demonstrated that parenteral feeding may be important in improving overall outcome (Derde et al. (2010) Crit. Care Med 38:602-611. Compared to starvation, a small dose of parenteral feeding in critically ill animals decreased muscle catabolism and did not induce significant lethality. A higher dose of parenteral feeding however holds risk of death, which thus reflects a trade-off for improved muscle preservation. As soon as hyperglycemia is allowed to develop, a higher lethality precludes any benefit from parenteral feeding (Derde et al. Crit. Care Med 2010).

Indeed, parenteral feeding has also disadvantages, one of which is development of hyperglycemia, which, if left untreated, leads to increased mortality, multiple organ failure and muscle breakdown. Our previous research indicates that even brief cellular hyperglycemia and nutrient overload exerts direct toxic cellular effects in the setting of critical illness, leading to these disastrous effects (Van den Berghe et al., (2001) N Engl J Med 345, 1359-1367; Van den Berghe et al. (2006) N Engl J Med 354, 449-461; Vlasselaers et al. (2009) Lancet 373, 547-556; Ellger et al. (2006) Diabetes 55, 1096-1105; Vanhorebeek et al. (2005). Lancet 365: 53-59, Vanhorebeek et al. (2009) Crit. Care Med 37, 1355-1364 and Vanhorebeek at al. (2009) Kidney Int 76, 512-520). Prevention of hyperglycemia in the critically ill, however, has shown to be difficult to achieve (Finfer et al. (2009) N Engl J Med 360, 1283-1297), specifically since there is a risk of hypoglycemia, which could counteract any benefit.

We now demonstrate here that such lethal effects of parental feeding and hyperglycemia in critically ill animals can be abrogated by administration of spermidine.

### Example 2

### Induction of Critical Illness in a Rabbit Animal Model

In our animal model, critical illness was induced by placing intravascular catheters, selectively destroying pancreatic β-cells by alloxan, followed by burn injury. As mentioned, this model revealed the dynamic endocrine and metabolic changes characteristic of human critical illness, including hyperglycemia and endogenous insulin deficiency. Alloxan is a toxic glucose analogue, which selectively destroys insulin-producing cells in the pancreas when administered to rodents and many other animal species. The administration of alloxan was necessary to control both blood glucose and plasma insulin levels independently. The application of the burn wound is done 48 hours after alloxan-injection, at which time alloxan has done irreversible damage to the β-cells (selective β-cell necrosis, phase 4 after alloxan-injection). After imposing a burn wound, animals were brought to hyperinsulinaemia, because this reflects most the human situation of critical illness. Non-injured, healthy rabbits served as control.

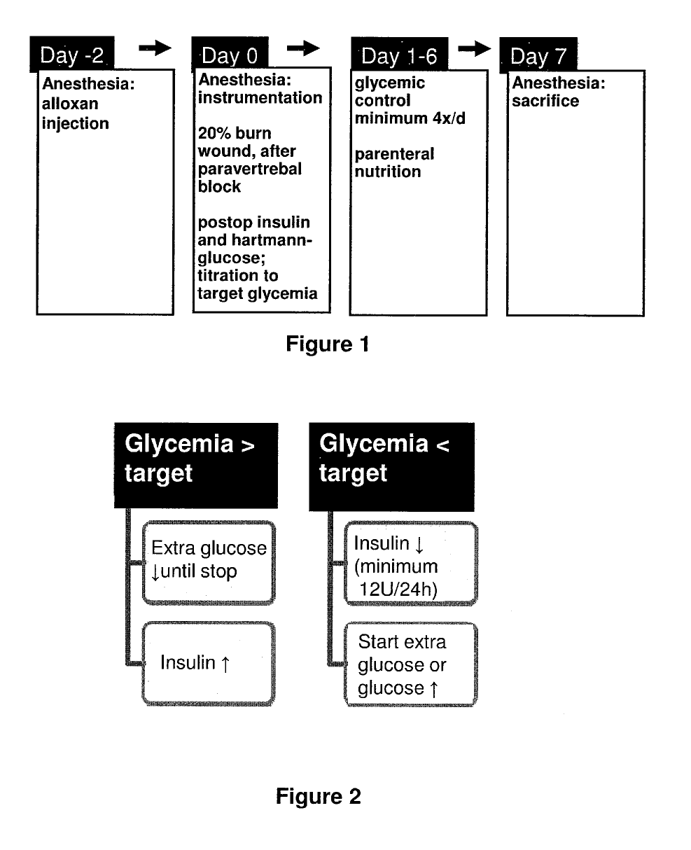

At 09:00±1 h of Day −2 (FIG. 1), animals were weighed, and randomized into three groups by sealed envelopes: group 1 (Burn/Hyperglycemia), group 2 (Burn/Normoglycemia), and group 3 (Control). The protocol was designed to reach at least a target of eight rabbits per group surviving until day 7. Under general anesthesia (30 mg/kg ketamine i.m. [Imalgene 1000; Merial, Lyon, France]; 0.15 ml/kg medetomidine i.m. [Orion, Espoo, Finland]), an ice-cold 10% solution of alloxan-monohydrate (150 mg/kg; Alloxan; Sigma-Aldrich, Bornem, Belgium) was injected slowly via a marginal ear vein. Afterward, the animals had free access to regular rabbit chow and drinking water enriched with glucose to face alloxan-induced acute hypoglycemia. Control rabbits were left untouched in the cage, had free access to regular rabbit chow, hay and received water and hay ad libitum.

At 10:00±1 h of Day 0 (FIG. 1), glycemia was measured in the burn groups to confirm hyperglycemia after alloxan (irreversible phase 4 after alloxan-injection). When glycemia exceeded 300 mg/dl, animals were considered eligible for the study. Under general anesthesia (see above) supplemented with 1.5 volume % isoflurane (Isoba Vet.; Schering-Plough, Brussels, Belgium) inhalation, animals were shaved and catheters were placed into the right jugular vein for intravenous infusion (4F; Vygon, Ecouen, France) and into the right carotid artery for blood sampling (5 Ch; Sherwood Medical, Tullamore, Ireland). A paravertebral block (5 ml Xylocaine 1%; Astra Zeneca, Brussels, Belgium) was performed and a full thickness burn injury of 20% body-surface area was imposed. Animals were then fitted to a homemade jacket to secure catheters and immediately returned to their cages. Continuous fluid resuscitation (16 ml/h Hartmann solution [Baxter, Lessiness, Belgium] supplemented with 25 g glucose/500 ml) was started via a volumetric pump (Infusomat secura; B.Braun, Melsungen, Germany) using a homemade swiffle device to allow free moving in the cage. Insulin (Actrapid; Novo Nordisk, Begsvaerd, Denmark) was continuously administered intravenously via a syringe pump (Perfusor secura; B.Braun), at a minimum dose of 4 U/kg/24 h. The two preset levels of blood glucose were achieved by adjusting a continuous glucose infusion (50% glucose via a syringe pump; Baxter) supplementing basal glucose intake (FIG. 2). Glycemic target was 80-110 mg/dl in the normoglycemic group and 300-315 mg/dl in the hyperglycemic group. Burn-injured animals were deprived of regular rabbit chow and received water and hay ad libitum. In the evening, a supplementary dose of piritramide was given subcutaneously (0.2 mg/kg Dipidolor; Janssen-Cilag, Beerse, Belgium). Control rabbits were left untouched in the cage until day 7, had free access to regular rabbit chow, and received water and hay ad libitum.

### Example 3

### Measurement of Spermidine Levels in Critically Ill Rabbits

At 13:00 1 h of Day 1. (FIG. 2), Hartmann solution was replaced by parenteral nutrition infused at 10 ml/h. We chose total intravenous nutrition because this is the only way to assure equal nutrient intake of the rabbits. Parenteral nutrition contained 35% Clinomel N7 (Baxter; Clinitec, Maurepas Cedex, France), 35% Hartmann solution, and 30% glucose 50%. All intravenous infusions were prepared daily under sterile conditions and weighed before and after administration for exact quantification of intake.

Parenteral nutrition was changed daily at 13:00±1 h of Days 2-7 (FIG. 2) at which time the amount of parenteral nutrition and supplementary glucose, and the amount of insulin given was recorded.

At 14:00±1 h of Day 7 (FIG. 2), animals were anesthetized using half of the above mentioned dose of anesthetics intravenously, and the animals were weighed. After tracheostomy, animals were normoventilated (small animal ventilator KTR4; Hugo Sachs, March-Hugstetten, Germany). Anesthesia was supplemented with 1.5 volume % isoflurane inhalation and 0.15 mg/kg piritramid i.v. Arterial blood pressure and central venous pressure (CVP) were monitored from the indwelling lines. Animals were sacrificed by cutting out the heart.

Peroperative on day −2 (before injecting alloxan), day 0 (after placing catheters), and thereafter daily at 09:00±1 h, arterial blood was sampled and immediately analyzed on a blood gas analyzer (ABL725, Radiometer Copenhagen, Denmark) to quantitate pH, hemoglobin, electrolytes, lactate, and glucose. After imposing the burn wound, glucose was measured minimum four times daily, and additional glucose measurements were carried out whenever blood glucose was unstable to allow tight adjustment of glucose/insulin infusion (FIG. 2). Supplementary, on day −2 (before injecting alloxan), day 0 (after placing catheters), and thereafter daily at 09:00±1 h, 3 ml blood was collected and centrifuged for 10 min at 10,000 rpm, and plasma was stored at −80° C. until further analysis. Healthy control rabbits underwent only 1 blood sampling (for blood gas analysis and plasma storage), i.e. before induction of anesthesia on day 7. Sampling was performed by puncturing the central ear artery. Urine was collected in a bucket under every cage. Daily at 13:00±1 h, 3 ml urine was sampled and stored at −80° C. until further analysis, and total urinary volume was recorded. On day 7, all organs were sampled under anesthesia and stored at −80° C. until further analysis.

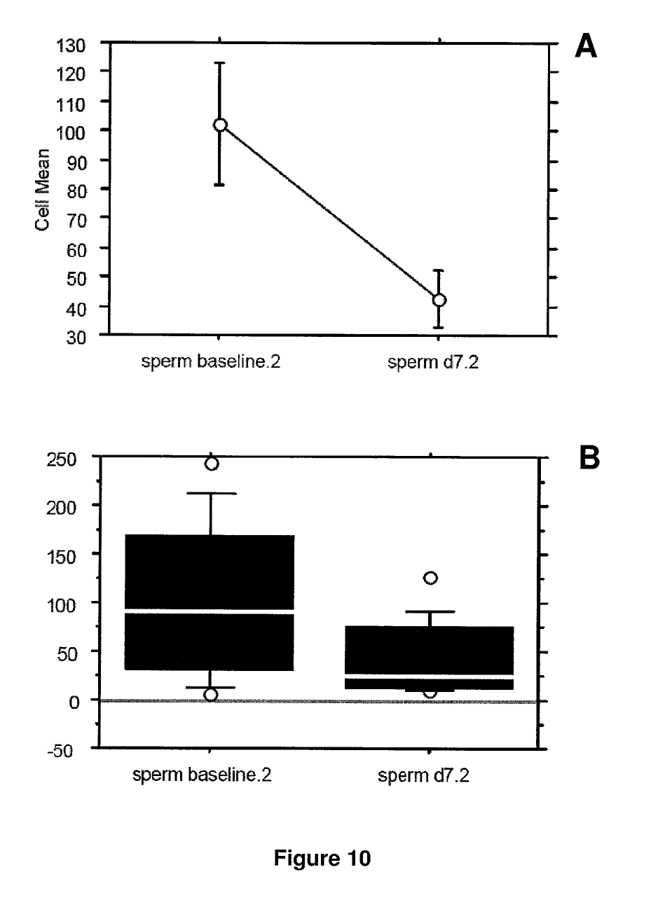

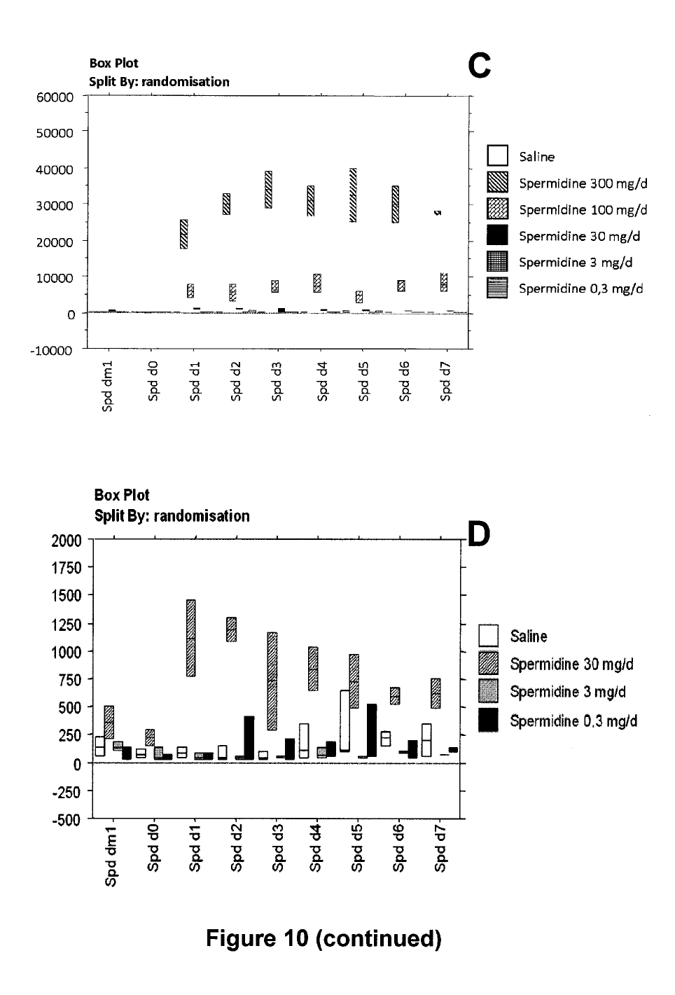

48 hours after alloxan injection, all animals had glucose levels >300 mg/dl, confirming permanent diabetic status. At day 7, spermidine levels in plasma of critically ill rabbits were significantly different compared to spermidine levels in plasma of healthy control rabbits (FIG. 9, FIG. 10). Day 7 spermidine levels of hyperglycemic rabbits were significantly different than levels of normoglycemic counterparts. Likewise, spermidine levels in tissue were significantly different in critically ill rabbits, compared to healthy controls. Hyperglycemic rabbits had significantly different tissue spermidine levels than normoglycemic rabbits.

In this animal model of critical illness, a spontaneous decrease in spermidine levels occurred along the time course of illness (measured by mass spectrometry; FIG. 9). A restoration or maintenance of spermidine levels to normal or slightly supranormal levels by exogenous administration of spermidine can improve organ function and survival of critical illness by better clearance of damaged organelles (mitochondria), as shown in the following experiment. Even severe mitochondrial insults, such as evoked by hyperglycemia and possibly aggravated by parenteral nutrition, can be survived when autophagy can be stimulated.

### Example 4

### Effects of Spermidine Administration in Critical Illness

The effects of spermidine administration were investigated in parentally fed, hyperglycemic, burn-injured rabbits. The rabbits were purchased from a local rabbitry and weighed approximately 3-3.5 kg. The burn injury experiments were performed analoguous to those described above. After imposing the burn wound, animals received a continuous infusion of saline or spermidine (low dose ranging from 0.3-3 mg/day or high dose ranging from 30-300 mg/day). Hence, we tested a dose range of spermidine approximately from 0.01 to 100 mg/kg/day.

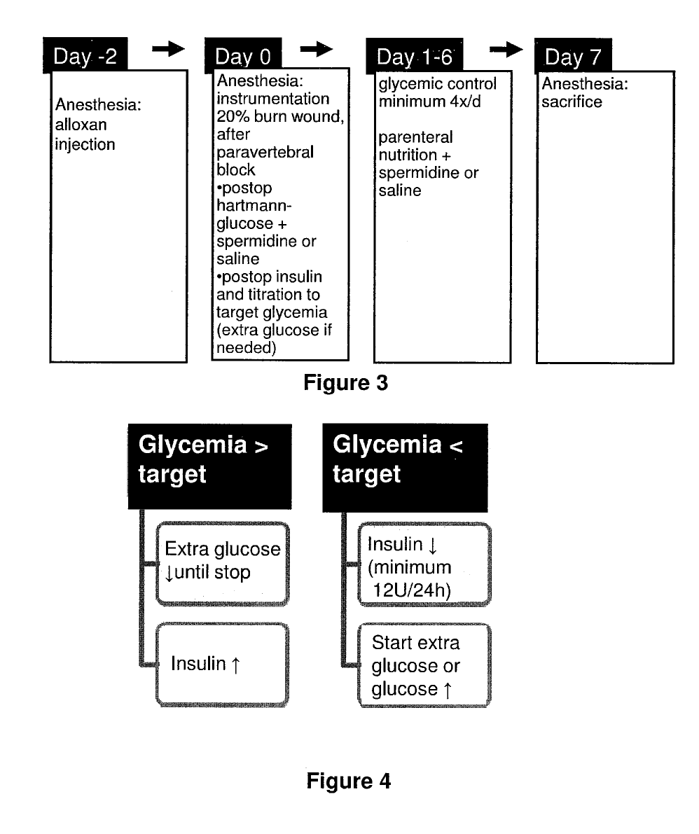

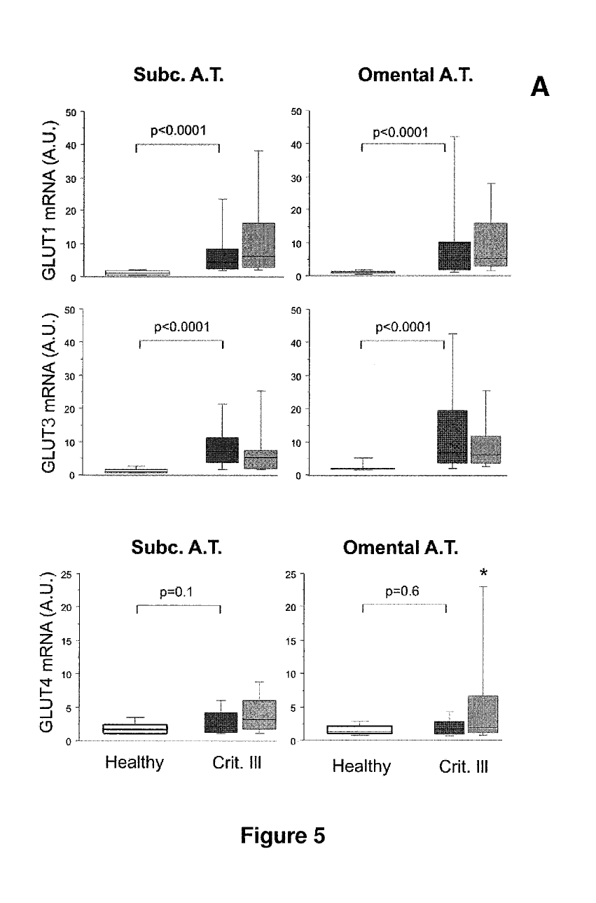

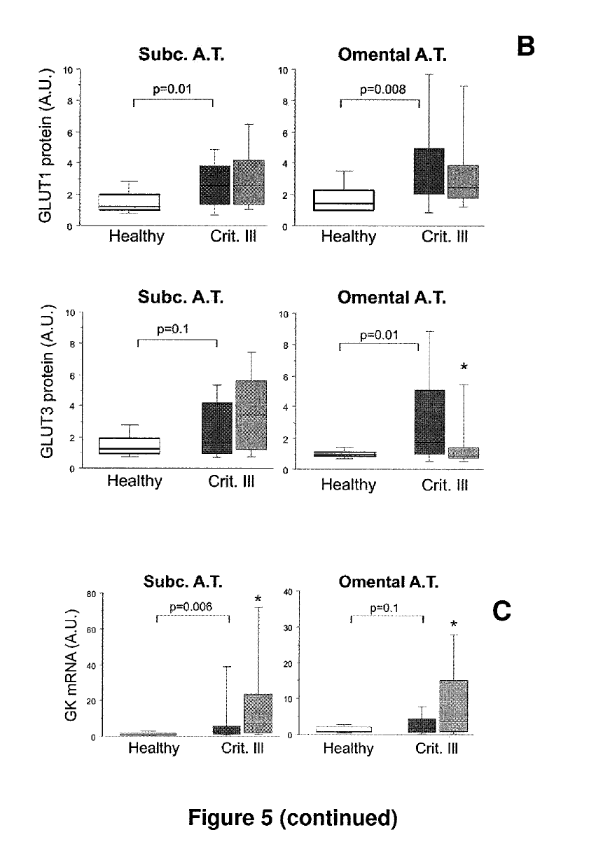

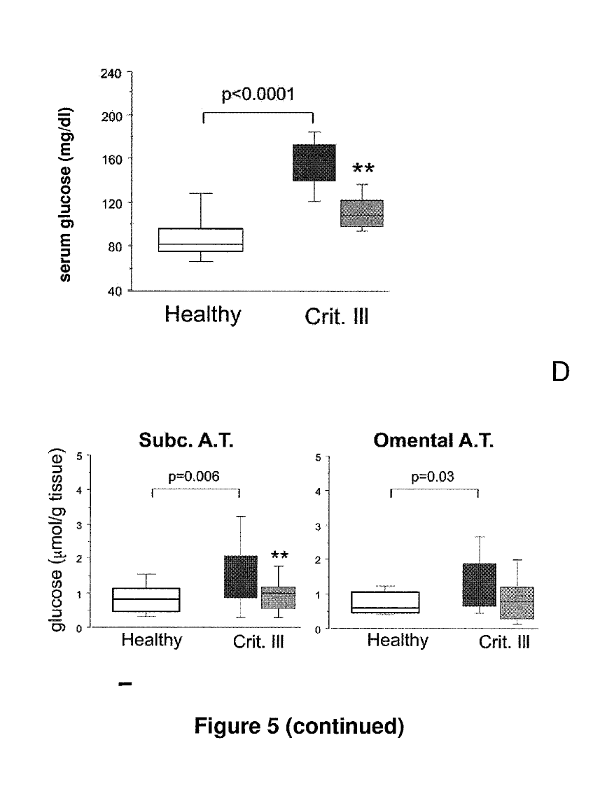

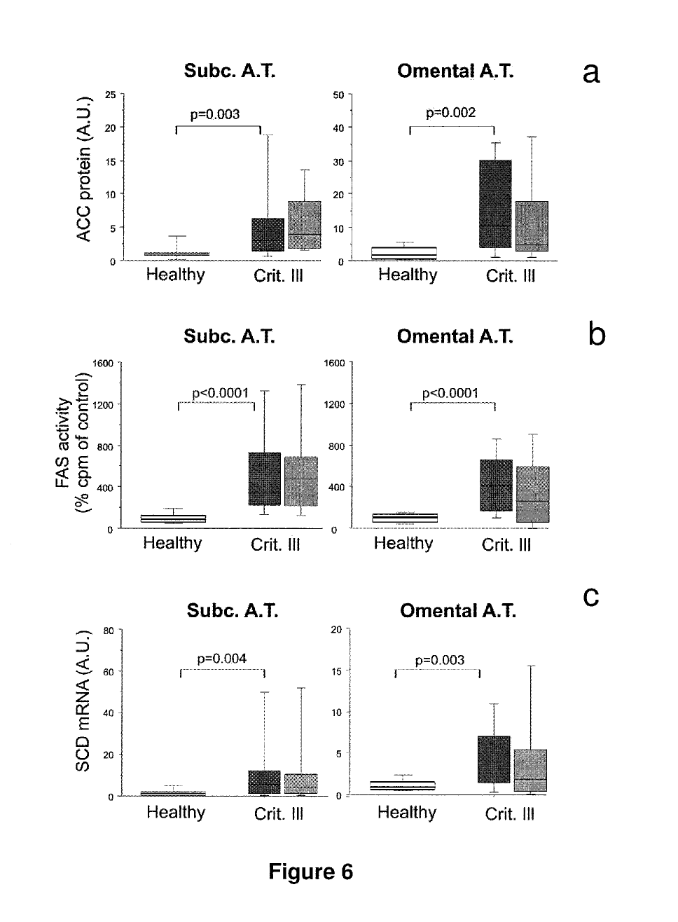

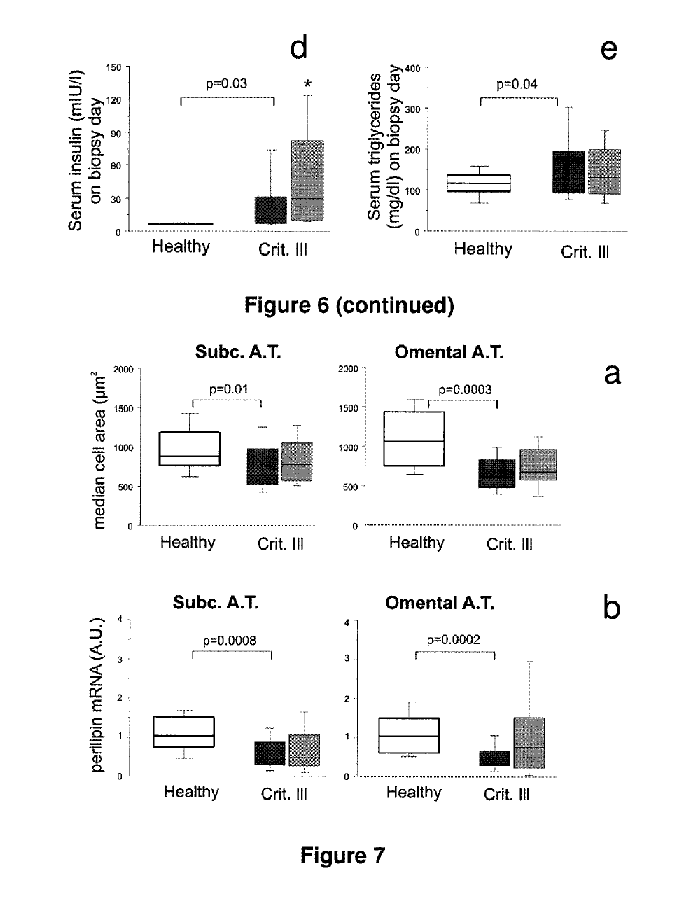

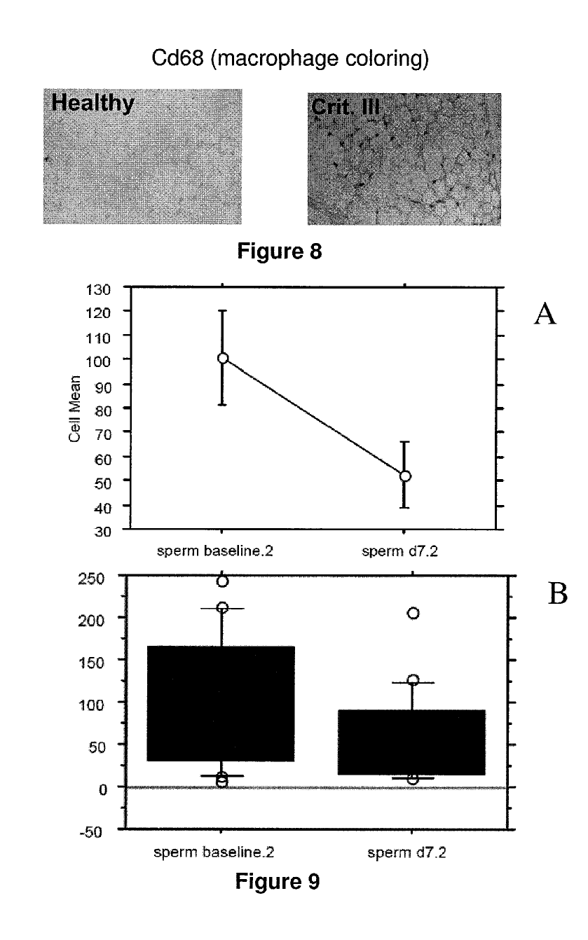

At 09:00±1 h of Day 2 (FIG. 3) animals were weighed, and under general anesthesia (30 mg/kg ketamine i.m. [Imalgene 1000; Merial, Lyon, France]; 0.15 ml/kg medetomidine i.m. [Orion, Espoo, Finland]), an ice-cold 10% solution of alloxan-monohydrate (150 mg/kg; Alloxan; Sigma-Aldrich, Bornem, Belgium) was injected slowly via a marginal ear vein. Afterward, the animals had free access to regular rabbit chow, hay and drinking water enriched with glucose to face alloxan-induced acute hypoglycemia.

At 10:00±1 h of Day 0 (FIG. 3), glycemia was measured to confirm hyperglycemia after alloxan (irreversible phase 4 after alloxan-injection). When glycemia exceeded 300 mg/dl, animals were considered eligible for the study. Under general anesthesia (see above), supplemented with 1.5 volume % isoflurane (Isoba Vet.; Schering-Plough, Brussels, Belgium) inhalation, animals were shaved and catheters were placed into the right jugular vein for intravenous infusion (4F; Vygon, Ecouen, France) and into the right carotid artery for blood sampling (5 Ch; Sherwood Medical, Tullamore, Ireland). A paravertebral block (5 ml Xylocaine 1%; Astra Zeneca, Brussels, Belgium) was performed and a full thickness burn injury of 20% body-surface area was imposed. Animals were then fitted to a homemade jacket to secure catheters and immediately returned to their cages. The animals were then randomized into six groups by sealed envelopes: group A (Spermidine 300 mg/day), group B (Spermidine 100 mg/day), group C (Spermidine 30 mg/day), group D (Spermidine 3 mg/day), group E (Spermidine 0,3 mg/day), or group F (Saline). The vials containing spermidine and saline were prepared in a sterile way by the hospital pharmacy, and the investigators were blinded to what the animals received.

Continuous fluid resuscitation (16 ml/h Hartmann solution [Baxter, Lessiness, Belgium] supplemented with 25 g glucose/500 ml) was started via a volumetric pump (Infusomat secura; B.Braun, Melsungen, Germany) using a homemade swivel device to allow free moving in the cage. Insulin (Actrapid; Novo Nordisk, Begsvaerd, Denmark) was continuously administered intravenously via a syringe pump (Perfusor secura; B.Braun), at a minimum dose of 2 U/kg/24 h. The preset levels of blood glucose were achieved by adjusting a continuous glucose infusion (50% glucose via a syringe pump; Baxter) supplementing basal glucose intake (FIG. 2). Glycemic target was 300-315 mg/dl. Animals were deprived of regular rabbit chow and received water and hay ad libitum. Animals received a continuous infusion of saline or spermidine via a syringe pump. In the evening, a supplementary dose of piritramide was given subcutaneously (0.2 mg/kg Dipidolor; Janssen-Cilag, Beerse, Belgium).

### Example 5

### Effects of Spermidine Administration in Critical Illness

At 13:00±1 h of Day 1 (FIG. 4), Hartmann solution was replaced by parenteral nutrition infused at 10 ml/h. We chose total intravenous nutrition because this is the only way to assure equal nutrient intake of the rabbits. Parenteral nutrition contained 35% Clinomel N7 (Baxter; Clinitec, Maurepas Cedex, France), 35% Hartmann solution, and 30% glucose 50%. All intravenous infusions were prepared daily under sterile conditions and weighed before and after administration for exact quantification of intake.

Parenteral nutrition was changed daily at 13:00±1 h of Days 2-7 (FIG. 4), at which time the amount of parenteral nutrition and supplementary glucose, the amount of spermidine/saline, and the amount of insulin given was recorded.

At 14:00±1 h of Day 7 (FIG. 4), animals were anesthetized using half of the above mentioned dose of anesthetics intravenously, and the animals were weighed. After tracheostomy, animals were normoventilated (small animal ventilator KTR4; Hugo Sachs, March-Hugstetten, Germany). Anesthesia was supplemented with 1.5 volume isoflurane inhalation and 0.15 mg/kg piritramid i.v. Arterial blood pressure and central venous pressure (CVP) were monitored from the indwelling lines. Animals were sacrificed by cutting out the heart.

Peroperative on day −2 (before injecting alloxan), day 0 (after placing catheters), and thereafter daily at 09:00±1 h, arterial blood was sampled and immediately analyzed on a blood gas analyzer (ABL725, Radiometer Copenhagen, Denmark) to quantitate pH, hemoglobin, electrolytes, lactate, and glucose.

From day 0 on, glucose was measured minimum four times daily, and additional glucose measurements were carried out whenever blood glucose was unstable to allow tight adjustment of glucose/insulin infusion (FIG. 4). Supplementary, on day −2 (before injection of alloxan), day 0 (after placing catheters), and thereafter daily at 09:00±1 h, 3 ml blood was collected and centrifuged for 10 min at 10,000 rpm, and plasma was stored at −80° C. until further analysis. Urine was collected in a bucket under every cage. Daily at 13:00±1 h, 3 ml urine was sampled and stored at −80° C. until further analysis, and total urinary volume was recorded. On day 7, all organs were sampled under anesthesia and stored at −80° C. until further analysis.

A detailed measurement by mass spectrometry of spermidine levels in plasma is presented in FIGS. 10 B and C (detail of FIG. 10B). Herein plasma levels (ng/ml) were measured from one day before application of the burn injury resulting in a critically ill condition up to 7 days after application of the injury.

Whereas low doses of spermidine (0.3-3 mg/day) had minimal or no effect on the plasma spermidine concentration, higher doses (30-300 mg/day) clearly maintained or increased the plasma spermidine to (supra)normal values.

A beneficial effect of spermidine was observed on overall survival, and the organ function (clinical, biochemical, morphological/cyto- and histopathological) and metabolic, inflammatory/immunological and cellular pathways were improved or restored. Thus, spermidine administration during critical illness could restore plasma and tissue levels of spermidine. Spermidine administration during critical illness resulted in decreased mortality, improvement of organ function, and affected multiple metabolic, inflammatory/immunological and cellular pathways. Blocking the effects of spermidine with an analogue had the opposite effects on survival, organ function and other morbidity.

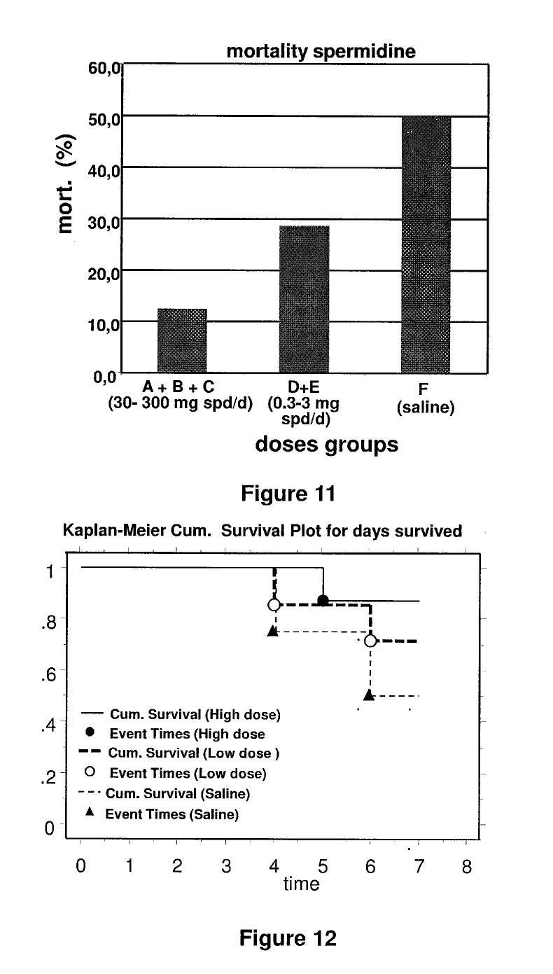

Spermidine administration, given to these parenterally fed hyperglycemic hyperinsulinemic critically ill animals, resulted in a marked improved survival rate, with the benefit most pronounced for the higher doses of spermidine (FIG. 11).

FIG. 11 shows the mortality of 3 groups of 8 animals receiving doses between 30 and 300 mg spermidine per day (group A, B and C). In these groups the mortality is 12.5%. 2 groups of 7 animals received doses between 0.3 and 3 mg spermidine per day (group D and E). In these groups the mortality is 28%. A control group (F) of 4 animals received a saline solution without spermidine. In this group the mortality is 50%. At any time point, there were more survivors in the groups receiving spermidine (FIG. 12).

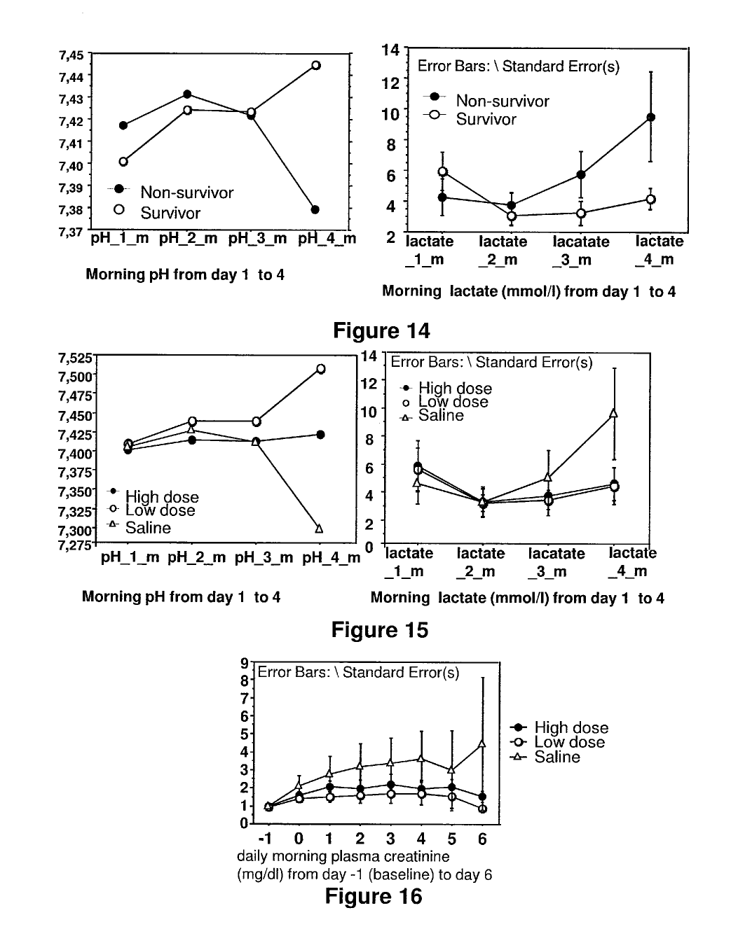

A considerable number of critically ill patients develop lactic acidosis as a result of increased anaerobic metabolism. The development of lactic acidosis (lowering of blood pH and an increase in lactate) is associated with poor outcome in patients, so blood pH and lactate can be used as markers of illness severity and predictors of outcome (Gunnerson et al. (2006) Crit. Care 10, R22; Mizock et al. (1992) Crit. Care Med 20, 80-93; Smith et al. (2001) Intensive Care Med 27, 74-83). Also in our rabbits, the development of lactic acidosis is associated with poor chances of survival. Already on day 3 of critical illness, some time before the first animals die because of illness, the plasma lactate of future non-survivors is different from lactate of survivors (FIG. 14) The evolution of pH and lactate over time is totally different between randomization groups. Rabbits who receive spermidine-infusion during illness are able to maintain a normal pH and normal lactate levels during their illness, and consequently have a higher survival. In rabbits receiving saline, lactate accumulates over time, pH decreases over time, and mortality is higher (FIG. 15).

Thus, the better survival in the spermidine groups was associated with a better preservation of blood pH and lactate, both being good markers of illness severity.

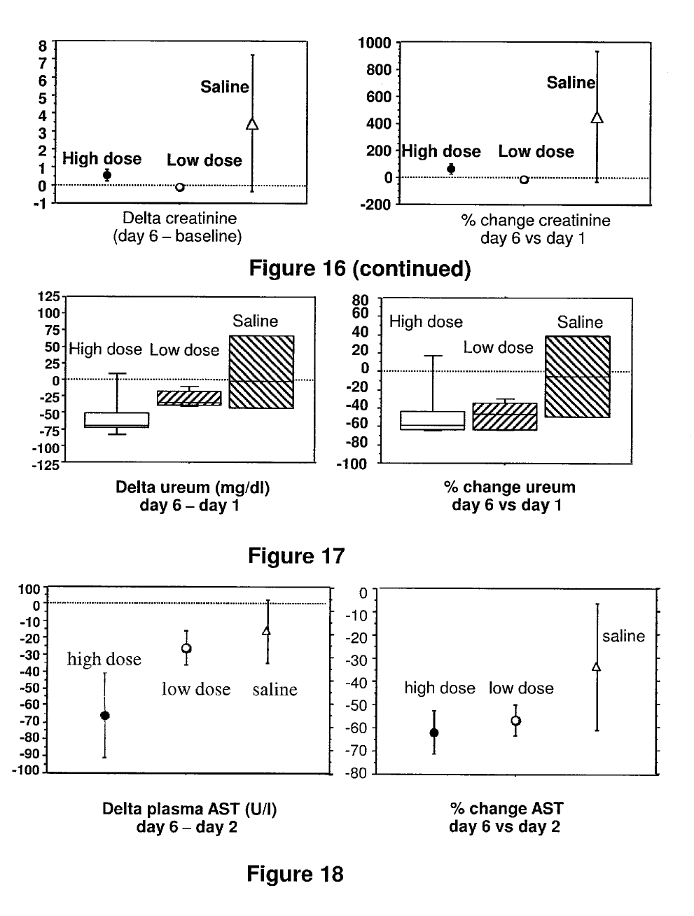

Plasma creatinine, a marker of kidney function, spontaneously rose in our animals, indicating the development of renal failure. This rise in creatinine could be prevented by administration of spermidine (FIG. 16A, 16B, 16C) (high dose: 30-300 mg/day, low dose 0.3-3 mg/day). Even low doses of spermidine resulted in renoprotection.

The rise in ureum, another marker of kidney dysfunction—as well as muscle wasting—could also be prevented by spermidine infusion (FIG. 17).

After the initial hit, our critically ill animals develop early liver dysfunction, as indicated by increased AST, a marker of liver dysfunction. In the days following, animals receiving spermidine have a rapid and profound decrease in AST-levels, indicating a rapid resolution of liver dysfunction after the initial hit. In animals receiving saline, recovery of liver function is hampered, as shown by less decrease in AST-levels (FIG. 18).

We also measured plasma and tissue levels of spermidine and other polyamines. Spermidine-administration could restore both plasma and tissue levels of spermidine, an effect already seen with doses of 1-200 mg spermidine per kg per day or spermidine in a range 5-120 mg/kg per day preferably 10-30 mg/kg per day.

Thus, counteracting the spontaneous decrease in spermidine, which we observed during critical illness (see above), strikingly reduced mortality and morbidity in our animal model of critical illness.

We are measuring mitochondrial function, as well as key proteins involved in autophagy in spermidine versus saline-treated critically ill animals. Preliminary results point to an improvement of mitochondrial function by spermidine administration, and a stimulation of autophagy by spermidine. These data indicate that spermidine administration can stimulate autophagy and hence lead to improvement of mitochondrial function via better clearance of damaged mitochondria. These data also support the concept that spermidine can counteract the feeding-induced suppression of autophagy.

### Example 6

### Effects of Spermidine Administration in Critical Illness

The results of the pilot study described above, were confirmed by determining the survival benefit in a statistically well-powered proof-of-concept outcome study. Based on the pilot study, two doses of spermidine (30 and 100 mg/day) were selected to continue with. In order to detect a 25% survival benefit compared to placebo (saline administration), with an alpha level of 0.05 and a power of 0.80, and after correction for multiple comparisons, 58 animals were included per group.

The experiment was performed as explained in detail above. An a priori planned interim analysis was performed after having included 15 animals per group, for selecting 1 spermidine-group to be completed next to the saline-group until 58 animals per group are included. After the interim analysis, the codes of the vials were changed, so that the proceeding of the study was again carried out blinded.

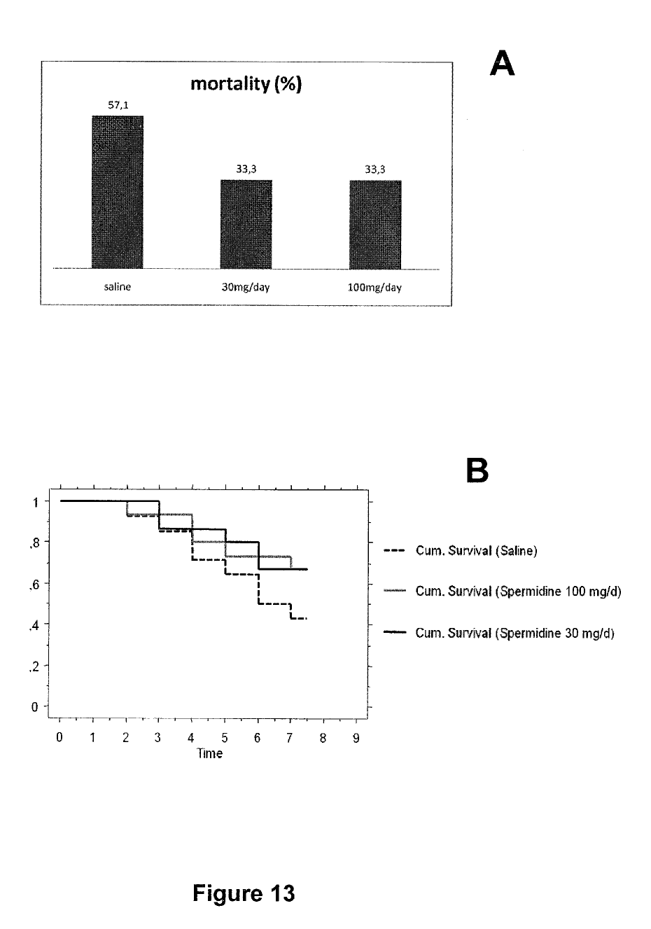

The interim analysis confirmed the beneficial results on mortality (FIGS. 13 A and B). Whereas the mortality, 7 days after application of the injury was high (>50%) in the placebo-group (saline), the mortality was much lower (about one third) by administrating spermidine. Both doses had an equal effect on mortality. The lowest dose (30 mg/day) was used for the continuation of the proof-of-concept study.

Plasma and tissue analyses have been performed on the animals that were included up to the interim analysis. Administration of spermidine 30 mg/d or spermidine 100 mg/d could maintain or increase the plasma spermidine concentration to similar (supra)physiological levels as in the pilot study. Interestingly, in sick rabbits receiving placebo (saline), the plasma spermidine levels were lower in rabbits who did not survive their illness, compared to survivors. Simultaneously, spermidine degradation products were increased in non-survivors. These findings support the hypothesis of the present invention of increased spermidine catabolism during critical illness, which could ultimately lead to a life-threatening spermidine deficiency.

Plasma markers of kidney and liver function were determined the rabbits used in the present experiment. The kidney function appeared protected by spermidine supplementation. Urea and creatinine, two markers of kidney dysfunction that accumulate when renal function decreases, were measured. In the present animal model of critical illness, kidney function spontaneously deteriorates severely over time, and this could be attenuated by spermidine administration. Indeed, the number of animals that developed a more than 3-fold rise in normal creatinine levels dropped from 25% in the saline-treated animals to 2.8% in the spermidine-treated animals (P 0.0161). Likewise, the number of animals that developed a more than 3-fold rise in normal urea levels dropped from 18.8% in the saline-treated animals to 2.8% in the spermidine-treated animals (P 0.0461).

Both kidney and liver of critically ill animals in the saline group showed signs of deficient autophagy. Both tissues of these animals displayed a marked accumulation of the autophagic flux marker p62, a protein that is normally degraded by autophagy and that accumulates in conditions of insufficient autophagy. Compared to saline-treated animals, the rise in renal p62 levels was attenuated by spermidine treatment, which points to a stimulation of autophagy by spermidine in the kidney. The stimulation of autophagy by spermidine in kidney was accompanied by a protection of the renal function. p62 accumulation appeared also important in the determination of liver function, as hepatic p62-levels showed a strong positive correlation with ALT and to a lesser extent with AST, both markers of liver dysfunction. Hence, these analyses corroborate the hypothesis of insufficient autophagy during critical illness as a contributor to organ failure and risk of death. The latter is further illustrated by the more pronounced p62 accumulation in kidney and liver of non-surviving animals, compared to animals that survived their illness. Hence, these findings provide a rationale for autophagy stimulation during critical illness.

### Example 7

### Toxicity of Spermidine

The toxicity of spermidine was assessed using a sequential up-down allocation technique. The administered dose of spermidine was determined by the response of the previous animal to a higher or lower dose (with incremental/decremental steps per 300 mg/d; minimum dose 600 mg/d). When the animal survived until day 4, this was considered as absence of acute toxicity. Mortality during the first 3 days was considered as acute toxicity. When there was no acute toxic effect, the next included animal would receive 300 mg spermidine/day more. When there was an acute toxic effect, the next included animal would receive 300 mg/day less (until the starting dose as a minimum). The starting dose was approximately 600 mg spermidine/day (twice the highest dose of the pilot study, which revealed no acute toxicity). The protocol was otherwise similar as in the experiments described previously.

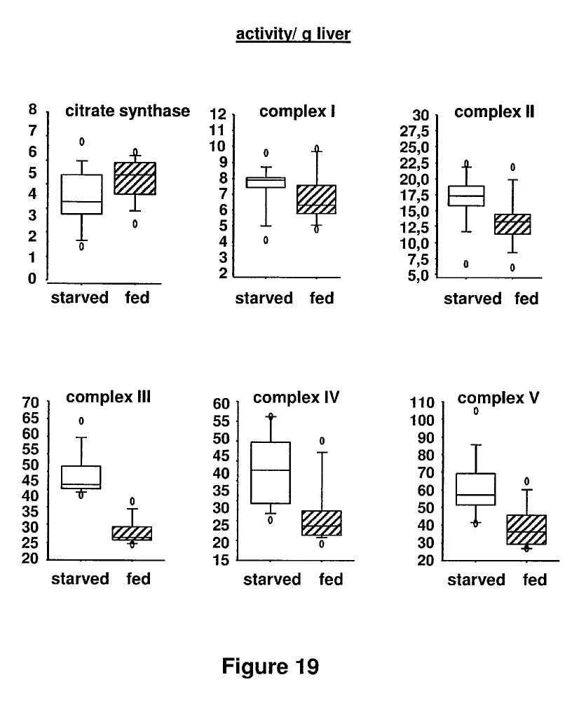

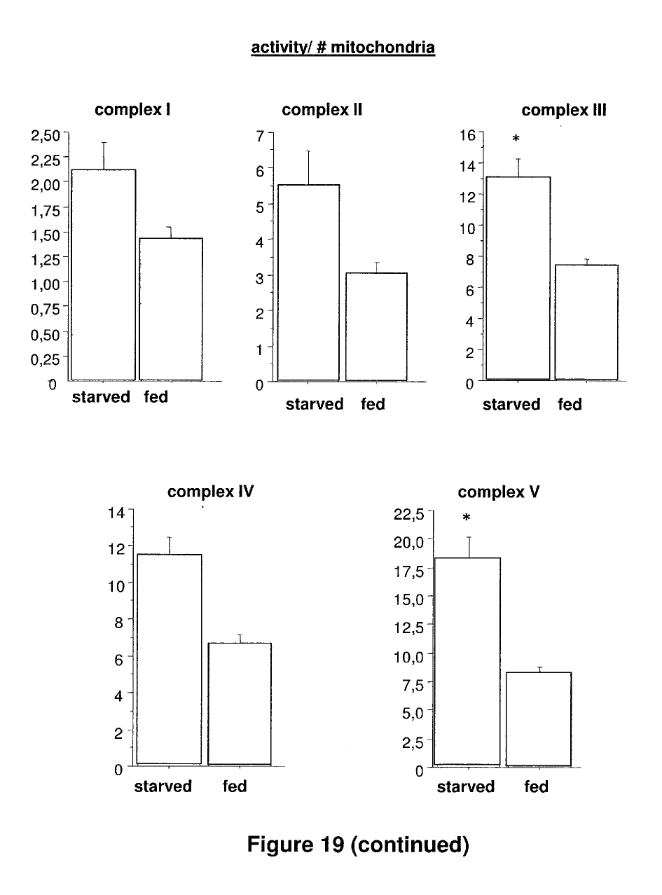

We included two animals in this toxicity study. Both animals received a continuous infusion of approximately 600 mg spermidine/day after induction of illness. Both animals died 28 hours after starting the spermidine infusion. Hence, we concluded to observe acute toxic effects with a dose of 600 mg/day (approx 200 mg/kg/day), which we did not observe with half of this dose (300 mg/day or approximately 100 mg/kg/day). The above examples show the effects of intravenous spermidine-supplementation in parentally fed critically ill animals (survival rate and toxicity). Although nutrition has the potential of benefit via a reduction of skeletal muscle catabolism in our animal model, some of the benefit is counteracted by inherent complications of feeding during critical illness, i.e. development of hyperglycemia and suppression of autophagy, whereby vital organ function may be impaired or the recovery of organ failure due to any initial insult (of whatever origin). This may explain the high mortality of ICU patients with organ failure. This may also explain why we found that starved critically ill animals (without receiving spermidine) have a better functioning of liver mitochondria than fed critically ill animals (also without receiving spermidine), because damaged mitochondria are better removed by starvation-induced autophagy (FIG. 19), supporting an indication for spermidine treatment. Indeed, administration of spermidine abrogates such secondary damage, also that induced by feeding, ultimately leading to improved overall outcome. Spermidine administration therefore emerges as an effective (preventive and therapeutic) strategy to enhance recovery and survival from multiple organ dysfunction and death in the critically ill.

### Example 8

### Effects of Spermidine Administration in Critical Illness

Critically ill patients requiring prolonged intensive care are characterized by a profound decrease of lean body mass but a preservation of adipose tissue. Furthermore, obese critically ill patients, with a BMI between 30 and 40, have a lower risk of death than patients with a normal BMI. Metabolic activity of adipose tissue in critical illness has hitherto not been studied. We hypothesized that critical illness, hallmarked by severe hyperglycemia, hyperinsulinemia and hypertriglyceridemia, changes adipose tissue substrate handling. We therefore studied glucose transport and metabolization, fatty acid metabolization and the anatomy of adipose tissue in subcutaneous and omental adipose tissue biopsies of 61 prolonged critical ill patients (taken minutes after death) and of 20 non-critically ill patients (taken during abdominal surgery).

The studied critically ill patients were included in a large randomized controlled trial on glucose control. Patients who had been randomly assigned to conventional insulin therapy (CIT) received insulin only when glucose concentrations exceeded 215 mg/dl, resulting in mean blood glucose of 157 mg/dl (hyperglycemia). IIT maintained blood glucose levels between 80 and 110 mg/dl resulting in mean blood glucose of 110 mg/dl (normoglycemia).

Glucose transporters (GLUT1, GLUT3) mRNA and protein expression was increased in adipose tissue of critically ill patients. Glucokinase mRNA expression was upregulated. Glucose tissue levels werey increased but G-6-P and glycogen adipose tissue levels were low in adipose tissue of critically ill patients, levels of acetyl CoA carboxylase and activity of fatty acid synthase was strongly upregulated. Also expression of stearoyl-coA desaturase, involved in the biosynthesis of mono-unsaturated fatty acids, was increased in critical illness. The cell area of adipocytes decreased in critical illness, as did the expression of perilipin, which is a lipid droplet coating protein in adipocytes. Furthermore, adipose tissue of more then 95% of the studied critically ill patients stained positive for CD68, a macrophage marker, while only 33% of the healthy control tissues did.

Together these results indicate a change in substrate handling in adipose tissue of critical ill patients. Our data suggest that glucose uptake in adipose tissue may be increased in critical illness, followed by an increased metabolization of glucose to fatty acids. Intensive insulin therapy only has very minor effects on these pathways. Concomitantly with increased lipogenesis, adipocyte cell number, rather then cell size, increased. The presence of macrophages in adipose tissue of critically ill patients might support an increased turnover of adipocytes. These changes turn adipose tissue into a functional ‘waist bin’ for toxic metabolites such as glucose during critical illness.

The experimental results show that low doses of spermidine (0.3-3 mg/day/rabbit) reduce the mortality of the animals in the experimenta model of critical illness but had minimal or no effect on the plasma spermidine concentration. These results also show that higher doses (30-300 mg/day/rabbit) reduce the mortality of the animals in the experimenta model of critical illness and clearly maintained or increased the plasma spermidine to (supra)normal values. An additional experiment including a large population of animals using 30 and 100 mg/day/rabbit of spermidine confirm the earlier observed recuced mortality.

A significant toxicity was observed when about 600 mg/day/rabbit was administered. The rabbits that have been used in these experiments typically have a weight of 3 kg, such that a significant reduction of mortality is obtained with a dosis from about 1 to 10, 33, up to 100 mg spermined/kg/day in rabbits.

Conversion factors are known to the skilled person to determine, starting from experimental data in rabbits, a suitable effective dosis for use in humans. Typical factors range from 15 to 60, typically from 30 to 60, whereby on a weight basis 15 to 60, typically from 30 to 60 times less compound is administered per kg to a human compared to a rabbit.

Accordingly it is estimated that depending on the conversion factor being used a suitable dosis for reducing mortality in a critically ill patient ranges from
0.07 mg/kg/day or 0.7 mg/kd/day to 2 mg/kg/day or to 7 mg/kg/day (conversion factor 15) or
0.03 mg/kg/day or 0.3 mg/kd/day to 1 mg/kg/day or to 3 mg/kg/day, (conversion factor 30), or
   0.02 mg/kg/day or 0.2 mg/kd/day to 0.5 mg/kg/day or to 1.7 mg/kg/day, (conversion factor 60).

In particular embodiments a suitable dosis for reducing mortality in a critically ill patient ranges from 0.5 to 2.5, 0.25 to 1.5 or 0.1 to 0.75 mg/kg/day for a human patient, depending on the conversion factor being used.

Based on the toxicity data in rabbits it is estimated that amounts of 13, 6 or 3 mg/kg/day for a human patient may be toxic.

The experimental data on rabbits make it plausible that a significant reduction of mortality can be obtained in a critically ill patient upon administration between 0.01, mg/kd/day/per human up to 10 mg/kg/day. Herein lower concentrations, from 0.01, 0.05, 0.1, 0.15 mg/kg/day in humans are suggested as lower boundaries of the effective range, ensuring a reduced mortality but not restoring endogenous plasma levels of spermidine. While higher concentrations, from 0.5, 1.0, 1.5, 2, 4, 5, 6, 7, 8 or 10 mg/kg/day in a human are suggested as higher boundaries of the effective range, ensuring a reduced mortality and restoring endogenous plasma levels of spermidine, with the risk of a toxic side effect of spermidine. However the toxic side effect of spermidine may be tolerable when mortality is reduced. Accordingly even higher doses of spermidine up to 20, 40, 60, 80 or even 100 mg/kg/day may be envisaged wherein the balance between reduced mortality and increased toxicity is evaluated.

The above doses which are derived from experimental data of spermidine to rabbits, can be used as a guideline to calculate doses for other polyamines.

### Example 9

### Materials and Methods for Experiments

### Experimental Animals and Determination of Thiol Groups in Mice Serum

Male and female C57BL/6 mice were purchased from the Institut für Labortierkunde und-genetik, Himberg, Austria. All animals were used at an age between 12 and 16 weeks. All mice were kept and treated according to institutional guidelines and Austrian law and the experiments were approved by the responsible governmental commission. For each group, one male and two female mice were housed singly and fed ad libitum with regular food (pellets) and spermidine was supplemented to drinking water in concentrations of 0.3 and 3 mM for 200 days. Controls were given pure drinking water. Drinking water was replaced every 2-3 days and spermidine freshly added from 1 M aqueous stock (spermidine/HCl pH 7.4), which was kept at −20° C. for no longer than one month. Food and body weight, calculated on a weekly basis, remained unaffected by supplementation of spermidine (data not shown), indicating that not calorie restriction could account for the observed effects. At the end of the experiment, the animals were anesthetized by ether inhalation, and exsanguinated by heart puncture. Peripheral blood was allowed to clot for 20 min, and serum was obtained by centrifugation at 200 g for 10 min. The spleens and livers (shock frozen in liquid nitrogen and stored at −80° C. upon further use) were immediately excised. Serum was used for determination of free thiol groups by Ellmans' reaction (Ellman (1959) Arch Biochem Biophys 82, 70-77 and Riener et al. (2002) Anal Bioanal Chem 373, 266-276 (2002).) as described previously (Schraml et al. (2007) Exp Gerontol 42, 1072-1078). Spleen weight, which was similar in all groups, indicated that all mice were of similar general health (data not shown).

### Extraction of Polyamines for LC/MS/MS Measurements

For acid extraction of polyamines from yeast cells (Balasundaram et al. (1991) Proc Natl Acad Sci USA 88, 5872-5876) culture equivalents of 20 OD₆₀₀ were washed three times with ddH₂O, resuspended in 400 μl ice-cold 5% TCA, and incubated on ice for one hour with vortexing every 15 min. Supernatants were neutralized with 100 μl of 2 M K₂HPO₄ and stored at −80° C. upon polyamine measurements using LC/MS/MS.

Extraction of polyamines from mice liver tissue was performed according to the freeze/thaw-method described by (Minocha et al. (1994)J Plant Growth Regul 13, 187-193) with slight modifications. Briefly, about 50-75 mg of mice liver tissue were semi-homogenized using Fisherbrand Disposable Pestle System (Fisher scientific) and polyamines extracted with 400 μl 5% TCA by three repeated freeze-thaw cycles. After extraction 100 μl of 2 M ammonium formiate were added to supernatants and stored at −80° C. upon polyamine measurements using LC/MS/MS.

### Polyamine Measurements Using LC/MS/MS

Polyamines were determined according to the method described previously by Gianotti et al. (V. Gianotti et al. (2008) J Chromatogr A 1185, 296-300.). All experiments were carried out on an Ultimate 3000 System (Dionex, LCPackings) coupled to a Quantum TSQ Ultra AM (ThermoFinnigan) using an APCI ion source. The system was controlled by Xcalibur Software 1.4. The stationary phase was a Sequent ZIC-HILIC column (150×2.1 mm, 3 μm, 100 Å). The elution solvent A was 50 mM ammonium formiate in ultra pure water and solvent B was acetonitrile. Separation was performed with 15% acetonitrile for 2 min. Thereafter, the acetonitrile content was linearly decreased to 5% over 2 min. After 1 min, acetonitrile content was increased to 15% for column equilibration. Flow rate was set to 300 μl/min.

Polyamines were detected in MRM mode using following transitions: spermidine (m/z 146->72, CE 34 eV), putrescine (m/z 89->72, CE 28 eV), bis(hexamethylene)-triamine as internal standard (m/z 216->100, CE 36 eV). Calibration standards were prepared by spiking extraction buffer with specific concentrations of spermidine, putrescine and internal standard. 20 μl of each sample were injected.

### Yeast Strains and Molecular Biology

Experiments were carried out in BY4741 (MATa his3Δ1 leu2Δ0 met15Δ0 ura3Δ0) and respective null mutants, obtained from Euroscarf. The double mutant Δiki3Δsas3 was generated according to Gueldener at al. (2002) Nucleic Acids Res. 30, e23 by using gene-specific URA3-knockout cassette, amplified by PCR with pUG72 as template (Lovaas & Carlin (1991) Free Radic Biol Med 11, 455-46.). The double mutant phenotype was confirmed using a strain generated by mating and sporulation of the respective single mutants (BY4742 Δiki3 MATα and BY4741 Δsas3 MATa). All spe1 double mutant strains were obtained through mating and sporulation of BY4741 Δspe1 with the respective BY4742 (MatΔ) single mutant strains. Single and double mutant strains were verified for correct gene deletion by PCR and further checked for consistent auxotrophies. Notably, at least three different clones of each generated mutant were tested for the survival plating during aging to rule out clonogenic variation. Strains were grown at 28° C. on SC medium containing 0.17% yeast nitrogen base (Difco), 0.5% (NH₄)₂SO₄ and 30 mg/l of all amino acids (except 80 mg/l histidine and 200 mg/l leucine), 30 mg/l adenine, and 320 mg/l uracil with 2% glucose (SCD). To demonstrate the complete requirement of polyamines for life span extension upon media alkalinization, experiments were carried out in polyamine-free SCD, obtained by sterile filtering and special treatment of glass ware as described (Balasundaram (1991) at al. Proc Natl Acad Sci USA 88, 5872-5876). To construct NHP6A-EGFP in pUG35-Ura (giving rise to a C-terminally tagged chimeric fusion protein under control of the met25-Promotor) the insert was amplified by PCR using genomic DNA from BY4741 as template and cloned into pUG35 using the EcoRI restriction site. The EGFP-ATG8 construct in pUG36-Ura (N-terminally tagged fusion protein) was similarly generated, using EcoRI and ClaI restriction sites.

### Yeast Survival Plating and Test for Cell Death Markers

For chronological aging experiments, cultures were inoculated from fresh overnight cultures to OD₆₀₀ of 0.1 (˜1·10⁶ cells/ml) with culture volume being 10% of flask volume and aliquots were taken out to perform survival plating at indicated time points (Herker, at al. (2004) J. Cell Biol. 164, 501-507. Survival at day 1 of wild type control cultures was set to 100% and other samples calculated accordingly. If not otherwise indicated, representative aging experiments are shown with at least three independent samples (as indicated) aged at the same time, which have been repeated at least twice with similar outcome. In case of experiments with Δspe1 (FIG. 24 A-E), all strains were inoculated to 5·10⁴ cells/ml. Upon deletion of both IKI3 and SAS3 we observed slight aggregation of cells possibly due to a defect in late budding events. Therefore, for calculation of survival rates in experiments using Δiki3Δsas3, cell numbers of each sample were determined after two pulse of sonication on ice with Sonifier 250 from Benson (Duty Cycle: 35; Output Control: 2.5). Tests for apoptotic (TUNEL and Annexin V staining) and necrotic (PI staining) markers as well as markers for oxidative stress (DHE staining) were performed as described in Dod et al. (1982) Eur J Biochem 121, 401-405). For quantifications using flow cytometry (BD FACSAria), 30,000 cells were evaluated and analyzed with BD FACSDiva software. Spermidine (S4139, Sigma, Austria) and putrescine (P5780, Sigma, Austria; 1 mM final concentration) were added to stationary cultures at day 1 of the aging experiments (24 h after inoculation). 1 M aqueous stock solution of spermidine was stored in one use aliquots at −20° C. for no longer than 1 month. For adjustment of extracellular pH (pHₑₓ) to 6 (±0.5), the required amount of sodium hydroxide was added 30 h after inoculation. The pHₑₓ was maintained at approximately 6 (±0.5) throughout the aging.

As a further marker for necrosis, nuclear release of the yeast HMGB1 homolog (Nhp6Ap) was monitored by epifluorescence microscopy of ectopically expressed chimeric fusion protein Nhp6Ap-EGFP. Therefore, yeast strains transformed with pUG35/NHP6A were grown on SCD lacking uracil and aged until indicated time points. Cells were washed once with PBS and directly applied to epifluorescence microscopy with the use of small-band EGFP filter (Zeiss) on a Zeiss Axioskop microscope in order to monitor intracellular localization of Nhp6A-EGFP. Expression during aging was verified by immunoblotting (data not shown). Notably, release of Nhp6A-EGFP to the extracellular space, which has been reported for mammalian HMGB1 (Lotze & Tracey (2005) Nat Rev Immunol 5, 331-342), could not be detected in yeast after 100× concentration of culture media (data not shown).

### Immunoblotting and Quantification of Histone Acetylation

Trichloroacetic acid whole-cell extracts were prepared according to the method described by Kao et al. (Howe at al. (2001), Genes Dev. 15, 3144-3154). Proteins were separated on 15% SDS-PAGE for Western blot analysis on PVDF membrane (Millipore) as described (Madeo et al. (2002) Mol Cell 9, 911-917) using CAPS buffer (10 mM 3-(Cyclohexylamino)-1-propanesulfonic acid, 10% methanol) for transfer of proteins. Blots were probed with the rabbit polyclonal antibody against histone H3 (ab1791, Abcam) (1:5,000), which served as a loading control for total histone H3, as well as the following histone H3 modification antibodies (Upstate Biotechnology): K56ac (1:6,000) (Recht at al. (2006) Proc Natl Acad Sci USA 103, 6988-6993); K18ac (1:10,000); and K9+14ac (1:10,000). Peroxidase-conjugated affinity-purified secondary antibody was obtained from Sigma. For quantification of relative acetylation blots were scanned using a densitometer (Molecular Dynamics, Model P.D. 300) and quantified with ImageQuant Version 5.1 (Molecular Dynamics). Band densities of acetylation specific blots were normalized to the respective densities of total histone H3 blots in order to obtain specific acetylation rates for each sample. Acetylation rates of wild type control cultures were normalised to 1 and the relative acetylation of each sample was calculated accordingly.

### Yeast Intracellular pH Measurement

Intracellular pH (pHᵢ) of aging yeast cells was assessed by FACS analysis of cells stained with the pH-dependent fluorescent dye SNARF-4F (Invitrogen, Austria), following the method described by Valli et al. (Valli et al., (2005) Appl Environ Microbiol 71, 1515-152) with slight modifications. The dye is applied as its acetomethyl ester (SNARF-4F-AM) and needs to be activated by intracellular esterases. In order to ensure sufficient activation in aging cells the incubation time for dye loading was increased to 30 minutes.

### Spontaneous Mutation Frequency and Budding Index

Spontaneous mutation frequency was determined based on the appearance of mutants able to form colonies on agar plates containing 60 mg/l L-canavanine sulfate according to Fabrizio et al. (Fabrizio et al. (2004) J Cell Biol 166, 1055-1067). Mutation rates were calculated per 10⁶ living (colony forming on YEPD) cells. Budding index was assessed by counting the percentage of budded cells after 10 seconds of sonication on ice using Sonifier 250 from Benson (Duty Cycle: 35; Output Control: 2.5) in micrographs of no more than 40 cells. For each sample, at least 500 cells were evaluated.

### Oxygen Consumption

Oxygen consumption was directly determined in 1.7 ml of chronologically aged yeast cultures transferred to a recording chamber by measuring the decline of oxygen concentration under anaerobic conditions using an oxygen electrode. Slopes were calculated over 15 min within the linear decrease of oxygen (minute 2-17) and normalized to living cells as determined by plating on YEPD agar plates.

### Fractionation of “Upper” and “Lower” (Quiescence) Cells

Cells were cultured in SCD media as described in section on “Yeast Strains and Molecular Biology”. Percoll density gradient centrifugation was performed according to Allen et al. (Allen et al. (2006) J Cell Biol 174, 89-100). Cell counts for each fraction were determined using a CASY cell counter (Innovatis).

### Yeast Nuclear Extract Preparation

Yeast nuclei were isolated from 200 ml BY4741 wild type culture (grown for 24 h in SCD to stationary phase) as described previously (Buttner et al. (2007), Mol Cell 25, 233-246). Nuclear extract was prepared using nuclear extraction buffer from BioVision's Nuclear/Cytosol Fractionation Kit (Bio Vision, K₂₆₆-25) without DTT addition, according to the manufacturer's protocol. Incubation time was doubled to 80 min with vortexing every 8 minutes. Protein concentration was determined via Bradford, giving yields of approximately 1 mg/ml protein. Yeast nuclear extract was immediately subjected to HAT activity assays.

### HAT Activity Assay

For HAT-activity determination the commercially available HAT Activity Colorimetric Assay KIT from BioVision (Bio Vision K332-100) was employed. HAT assays were performed according to the manufacturer's protocol. In brief, assays were performed with each 15 μg of yeast nuclear extract or nuclear extract of HeLa-cells (Bio Vision K332-100-4), respectively. Spermidine was added at a final concentration of 100 mM 15 minutes after assay initiation. Development of tetrazolium dye was measured by absorption at 440 nm using a GeniosPro plate reader (Tecan). Background readings were done with samples without NADH generating enzyme, giving the nuclear extracts unspecific background activity and eliminate any possible negative effects of spermidine addition on the assay itself. For calculation of relative HAT activity linear regression over 100 minutes within the suggested assay time (minute 95 to 195) was performed to determine the slope of dye development. Regression coefficients of R²>0.99 were obtained. Calculated slopes of spermidine treated samples were compared to slopes of untreated samples which were set to a relative activity of 100%.

### Yeast RNA Isolation and Affymetrix Array Analyses

Total RNA extraction from chronologically aged yeast cells (with or without spermidine application) by glass bead disruption were performed using RNeasy MiniKit (Qiagen) according to the manufacturers' instructions. 10⁸ cells were used after shock freezing in liquid nitrogen and storage at −80° C. upon preparation. RNA of two independent aging experiments at day 3 and 10 of the aging experiment (biological replicates) was applied to Affymetrix Array Analyses.

Syntheses of cDNA and hybridization experiments were outsourced to the Microarray

Facility Tuebingen, Germany, an authorized Affymetrix Service Provider. Hybridization was done onto high-density oligonucleotide arrays Yeast Genome 2.0 (Affymetrix). Both, experimental and data analysis workflow were fully compliant with the MIAME 2.0 Standard. Annotation Data for the Yeast Genome 2.0 Array were supplied by Affymetrix Inc. Raw data were normalized with GCRMA (Wu et al. (2004) Journal of the American Statistical Association 99, 909-918) using CarmaWeb (Rainer et al. (2006) Nucleic Acids Res 34, W498-503). P-values were calculated with a paired T-Test comparing untreated (controls) with treated (spermidine supplemented) samples at the respective time points using TM4 MeV software (Saeed et al. (2003), Biotechniques 34, 374-378).

### Yeast Autophagy Measurements

Autophagy was monitored either by vacuolar localization of Atg8p using fluorescence microscopy of cells expressing an EGFP-Atg8 fusion protein (T. Kirisako et al. (1999) J Cell Biol 147, 435-446.) or by alkaline phosphatase (ALP) activity according to Kissova et al. (Kissova at al. (2004) J Biol Chem 279, 39068-39074) using BY4741 wild type strain transformed with and selected for stable insertion of pTN9 HindIII fragment (confirmed by PCR). In order to correct for intrinsic (background) ALP activity, BY4741 (without pTN9) have been simultaneously processed and ALP activity subtracted. For generation of EGFP-ATG8 constructs see section on Molecular Biology.

### Statistical Analyses

Statistical analyses were performed using Students T-Test (one-tailed, unpaired).

### Example 10

### Results

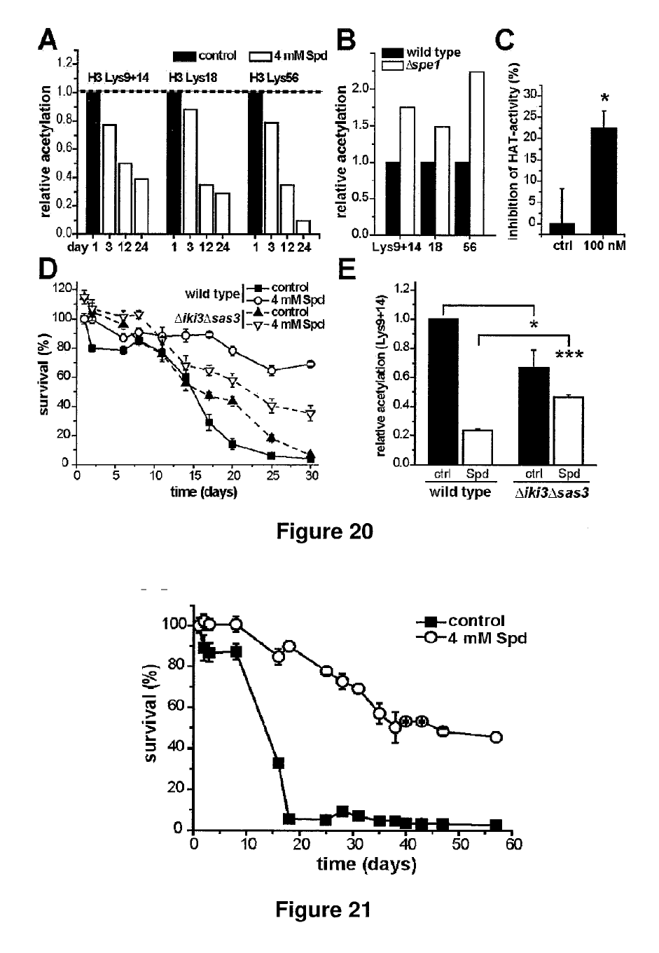

Histone H3 acetylation is regulated by intracellular polyamines in part mediated through Iki3p and Sas3p, as shown in FIG. 24 with: (A) Immunoblot of whole cell extracts of wild type cells chronologically aged to designated time points with (+) or without (−) spermidine application. Blots were probed with antibodies against total histone H3 or H3 acetylation sites at the indicated lysine residues; (B) Relative acetylation of histone H3 lysine 9+14 of Δspe1 cells compared to wild type cells chronologically aged to day 5 with (open bars) or without (closed bars) adjustment of pHₑₓ to 6. Data represent means±SEM of three independent experiments. **p<0.01; (C) Quantification (FACS analysis) of phosphatidylserine externalization and loss of membrane integrity using AnnexinV/PI co-staining performed at day 20 of the chronological aging experiment shown in (FIG. 20D). For each staining 30,000 cells were evaluated. ***p<0.001; (D) Immunoblot of whole cell extracts of wild type and Δiki3Δsas3 cells with (+) or without (−) spermidine application obtained at day 20 of the aging experiment shown in (FIG. 20D). Blots were probed with antibodies against total histone H3 or H3 acetylation sites at lysine 9+14 (Lys9+14).

Exogenous supply of spermidine to chronologically aging (BY4741 wild type) cells (at day 1) caused a drastic increase in yeast life span by a factor of up to 4 times, as determined in clonogenic assays that monitored the frequency of viable cells (FIG. 19B). Similar results were obtained using the wild type strain DBY746 (data not shown). Supplementation of spermidine led to a stable increase in the intracellular spermidine level in aging cells that otherwise would exhibit a decrease in spermidine levels (FIG. 21).

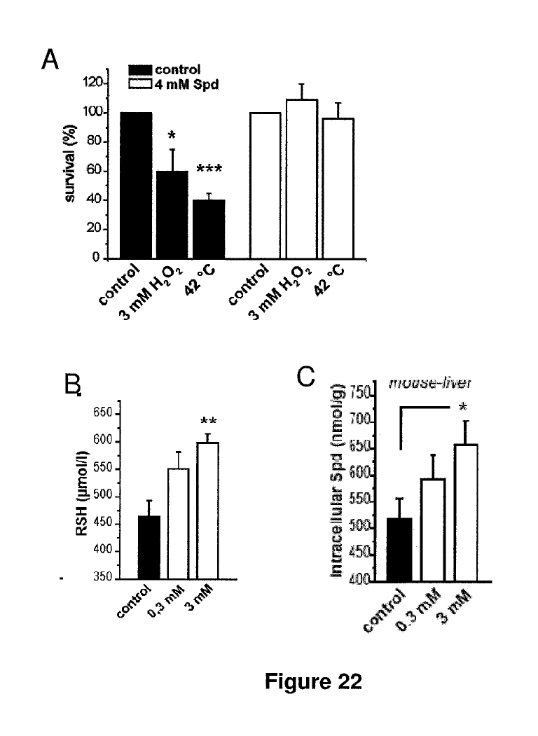

Aged yeast cells treated with spermidine did not only exhibit an increased life span, but also a strong resistance against stress inflicted by heat shock or H₂O₂ treatment (FIG. 22). Present invention also demonstrates that orally delivered spermidine is actively taken up and can be used to increase the intracellular levels of bioactive spermidine. One of the most widely accepted theories of aging is the free radical theory that explains aging by accumulating oxidative stress (Harman (1956) J Gerontol 11, 298-300). Consistently, in rodents the level of oxidative stress and protein damage increases with age, observable in the serum by a decline of free thiol groups (Schraml et al. (2007) Exp Gerontol 42, 1072-1078). Feeding mice with 3 mM spermidine (supplemented to drinking water) for 200 days increased the serum level of free thiol groups by ˜30%, indicative of reduced age-related oxidative stress (FIG. 22B). Notably, such an increase of free thiol groups is comparable to the natural decline that has been observed during the course of aging (between young and old rodents) (Schraml et al. dieted above). Again, intracellular spermidine levels were significantly increased by exogenous spermidine supplementation as determined in liver cells (FIG. 22C).

Next, we investigated the effect of polyamine depletion on aging, using a yeast strain deleted in SPE1 (Δspe1) and hence unable to synthesize polyamines. Polyamine depletion, as confirmed by measurement of intracellular spermidine (FIG. 18A), caused a drastic drop in yeast chronological life span (FIG. 24B), which can be restored by supplementation with spermidine or putrescine, the obligate precursor of spermidine (data not shown). Consistent with the free radical theory of aging (Harman (1956) J Gerontol 11, 298-300), we observed an enhanced accumulation of oxygen radicals upon disruption of SPE1, as measured by dihydroethidium (DHE) staining (FIG. 24C, 24D). An enhancement of radicals upon SPE2 deletion has also been shown in growing cells (Chattopadhyay et al. (2006) Yeast 23, 751-76). Since oxidative stress can cause apoptosis in yeast (Madeo et al., (1999) J Cell Biol 145, 757-767) we determined apoptotic markers of wild type and polyamine depleted Δspe1 cells. Surprisingly, the frequency of apoptotic events (that is cells that exhibit DNA-fragmentation detectable by TUNEL or phosphatidylserine externalization detectable with Annexin V) was not affected by SPE1 deletion (FIG. 24E). Instead, we observed an increase in necrotic, PI positive cells in Δspe1 cultures as compared to wild type controls (FIG. 24E). Accordingly, deletion of apoptotic effector molecules (including the yeast caspase, Yca1p; apoptosis-inducing factor, Aif1p; endonuclease G, Nuc1p; or the serine protease HtrA2/OMI, Nma111p) in the background of Δspe1 did not prevent the aging-associated death accelerated by polyamine depletion (data not shown). We therefore conclude that depletion of intracellular polyamines can precipitate premature chronological aging via non-apoptotic, possibly necrotic death of yeast cells.

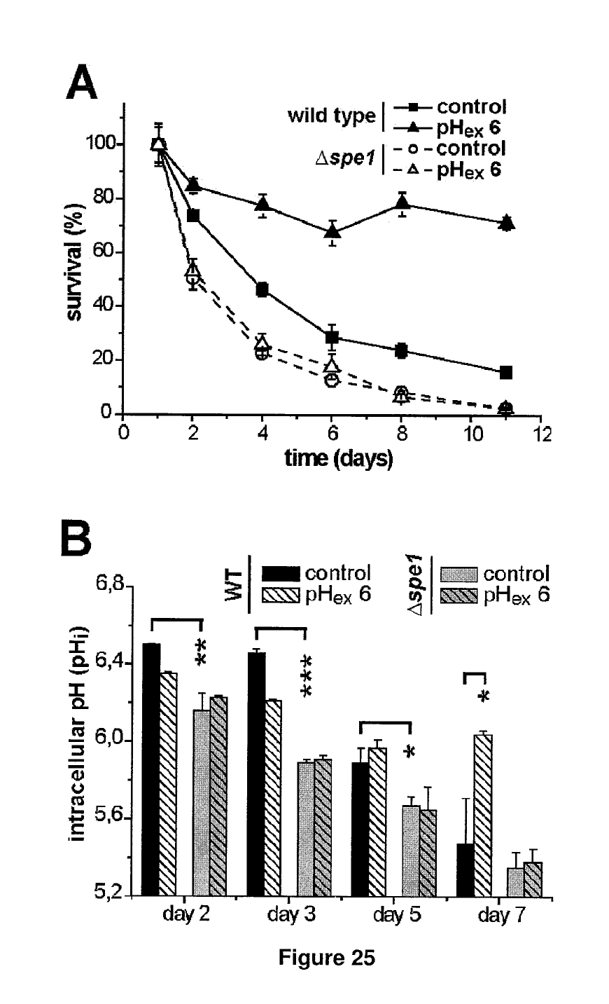

Very few studies have addressed the mechanisms of necrotic cell death in a systematic fashion. Using C. elegans as a model, it has been demonstrated that acidification of the cytosol is required for necrotic cell death, whereas alkalinization has a cytoprotective effect (Artal-Sanz et al. (2006) J Cell Biol 173, 231-239, Syntichaki et al. (2005) Curr Biol 15, 1249-1254). In yeast, adjustment of the extracellular pH from normally 3.5 to 6.5 not only extends chronological life span (Fabrizio et al., (2004) J Cell Biol 166, 1055-1067) but also stabilizes the intracellular pH in a polyamine dependent manner (FIG. 25B), corroborating the assumption that cytosolic acidification limits cellular life span. Since addition of 4 mM spermidine to chronologically aging yeast increases the extracellular pH to 6 (±0.5), we asked, if indeed intracellular polyamines (e.g. spermidine) were responsible for life span extension under these conditions. Making use of polyamine depleted cells (Δspe1), we demonstrate that the protective effect of external alkalinization on longevity, and thus of spermidine application, is strictly dependent on intracellular polyamines (FIG. 25A).

Consistent with an anti-necrotic effect of alkalinization in C. elegans, spermidine treatment procures a drastic reversion of age-associated necrosis in yeast.

Determination of cell death markers revealed that markers of necrosis and oxidative stress (DHE positivity) were drastically diminished upon spermidine treatment (FIG. 26C). In contrast, externalization of phosphatidylserine, an early apoptotic marker (Annexin V⁺PI⁻ cells), remained largely unaltered. Instead, loss of membrane integrity due to primary necrosis (PI positivity) and late apoptosis resulting in secondary necrosis (Annexin V⁺PI⁺) was reduced from 50% to less than 10% in spermidine-treated cultures as late as after 18 days of aging (FIG. 26C). We therefore conclude that death associated with chronological aging of yeast is mainly mediated by spermidine-inhibitable necrosis-like cell death.

As the extended life span of spermidine-treated cultures was neither associated with an increased mutation frequency nor a higher budding index (FIG. 27), we speculated that epigenetic modifications rather than genetic changes (such as the regrowth of death-resistant mutants) were responsible for the positive impact of spermidine on longevity. We could also exclude that a simple direct anti-oxidant effect of polyamines Lovaas & Carlin (1991) Free Radic Biol Med 11, 455-461) could account for the observed life span extension.

Global histone deacetylation, a key event of epigenetic chromatin modification, is associated with prolonged life span and healthy aging in a wide range of organisms (Longo et al. (2006) Cell 126, 257-268, Sauve et al. (2006) Annu Rev Biochem 75, 435-465). Since (de)acetylation of lysyl residues of histone H3 is critical for yeast longevity, at least during replicative aging (Imai et al. (2000) Nature 403, 795-800), we analyzed the effects of spermidine on the level of histone acetylation by means of a panel of specific antibodies that detect H3 acetylation at 4 different lysyl residues. The improved life span of aging wild type cells treated with spermidine correlated with hypoacetylation of histone H3 at all monitored acetylation sites (FIG. 20A, 23A). Conversely, premature death of aging SPE1-deleted cells was accompanied by hyperacetylation of histone H3 (FIG. 20B). These results hint to an obligatory role for polyamines in the regulation of histone acetylation during aging. In line with the strict requirement of polyamines for life span extension upon extracellular alkalinization, we observed hypoacetylation of the chromatin only in wild type, but not in SPE1 knockout cells upon alkalinization of the culture medium (FIG. 28B). These results suggest that global deacetylation and polyamines are connected to the extension of chronological life span in yeast.

As the role of the Sir2p deacetylase is well established in replicative aging (Longo (2006) Cell 126, 257-268), Lin et al. (2000) Science 289, 2126-2128), we tested its potential involvement in polyamine-promoted longevity during chronological aging. However, deletion of SIR2 did not abrogate the ability of spermidine to extend the chronological life span. Thus, the observed hypoacetylation during chronological life span extension is not due to the sole induction of Sir2p activity. Similarly, single deletion of all other known yeast sirtuins (HST1, HST2, HST3, HST4) did not affect longevity upon spermidine application (Table 2). This result is compatible with previous findings suggesting that chronological life span extension by calorie restriction is not mediated by Sir2p activity (Fabrizio et al., (2005) Cell 123, 655-667) nor by any of the other yeast sirtuins (Smith et al. (2007) Aging Cell 6, 649-662).

Theoretically, spermidine treatment could lead to hypoacetylation either via activation of histone deacetylases or via inhibition of histone acetyltransferases (HATs). Therefore, we determined the effects on aging of the disruption of 28 genes involved in histone (de)acetylation in the presence or absence of spermidine. The anti-aging (pro-survival) effect of spermidine was partially abrogated in two of these strains, namely Δiki3 and Δsas3 (data not shown, Table 2). Interestingly, the histone acetyl transferase Sas3p preferentially acetylates histone H3 at lysine 14 (Howe et al. (2001) Genes Dev 15, 3144-3154), one of the acetylation sites that is profoundly influenced by the availability of intracellular polyamines (see above). Deletion of IKI3, an essential subunit of the histone acetylating elongator complex, has been reported to reduce histone H3 acetylation at lysine 14 as well (Winkler et al. (2002) Proc Natl Acad Sci USA 99, 3517-3522). Consequently, we generated the double mutant Δiki3Δsas3 in an attempt to diminish these overlapping HAT-activities converging on lysine 14 of histone H3.

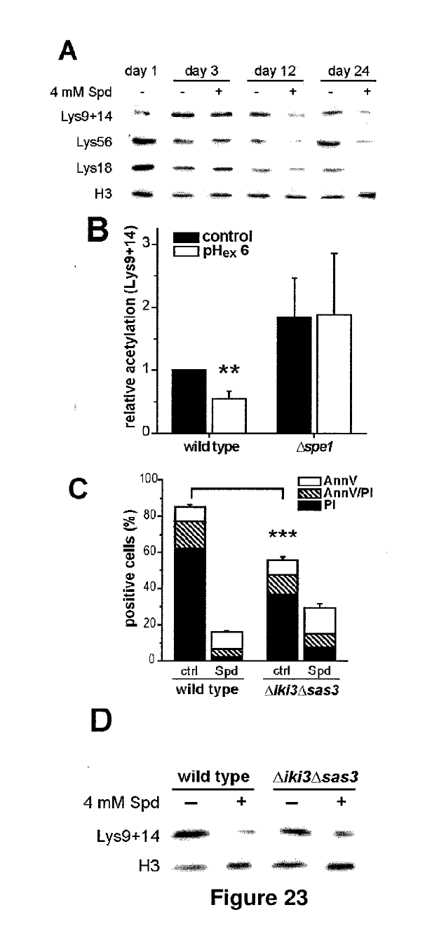

Chronologically aged Δiki3Δsas3 cells responded significantly less to the anti-aging effect of spermidine than wild type cells (FIG. 20D, p=0.002 for day 20). At day 20, spermidine treatment increased survival of wild type cells by 5-fold compared to only 1.3-fold for Δiki3Δsas3 cells, suggesting that Iki3p and Sas3p are, at least to some extent, required for the life span prolonging effects of spermidine. Moreover, the untreated double mutant showed an improved survival during chronological aging as compared to wild type controls (FIG. 20D, p<0.001 for day 20), indicating that histone acetylation activity is responsible for age-induced cell death. Accordingly, histone H3 acetylation was significantly reduced upon deletion of IKI3 and SAS3, and spermidine application barely reduced the level of acetylation in this mutant (FIG. 20E). Evaluation of cell death markers revealed that increased survival of Δiki3Δsas3 cells is clearly due to the inhibition of necrotic death (PI staining) while apoptotic markers (Annexin V) remained constant (FIG. 23C). The combined knockout of IKI3 and SAS3 did increase the life span of yeast cells, yet failed to mimic the life span prolongation of spermidine in quantitative terms, presumably because epigenetic aging processes are likewise regulated by more than just two proteins that modify the level of histone H3 acetylation on one lysyl residue. An in vitro assay for HAT-activity revealed that spermidine efficiently inhibited general HAT activity in extracts of isolated yeast and mammalian nuclei (FIG. 20C, data not shown). These results suggest that spermidine-mediated anti-aging effects are achieved via direct inhibition of HAT-activity.

Autophagy is believed to be essential for healthy aging and longevity, and the autophagy-regulatory Tor-pathway constitutes one of three highly conserved signaling pathways controlling aging of various organisms (Powers et al. (2006) Genes Dev 20, 174-184) FIG. 29).

Altogether, our results demonstrate that epigenetic regulation of necrotic death determines the chronological life span of yeast and that this epigenetic regulation is mediated by proteins involved in histone acetylation (such as Iki3p and Sas3p), which in turn are inhibited by spermidine. Additionally, histone (de)acetylation (and subsequent regulation of cell death) might also be regulated by polyamines depending on their acetylation state thereby directly modifying chromatin accessibility (Liu et al. (2005) J Biol Chem 280, 16659-16664). Consistently, we observed that deletion of PAA1, the sole known polyamine acetyl transferase (Liu et al. (2005) J. Biol Chem 280, 16659-16664), effectively shortens yeast chronological life span accompanied by enhanced ROS levels (FIG. 30).

We showed that spermidine strongly induces autophagy. Autophagy constitutes the major lysosomal degradation pathway recycling damaged and potentially harmful cellular material (such as damaged mitochondria). Of note, autophagy counteracts cell death and prolongs life span in various ageing models (Galluzzi et al. (2008) Curr Mol Med 8, 78-9). Therefore, inhibition of necrotic cell death by autophagy could facilitate the long-term survival of spermidine-treated cells.

It is generally accepted that hypoacetylation is a key event of gene silencing (Guarente (2000) Genes Dev 14, 1021-1026). Silencing might be concomitantly linked to lower metabolic rates causing less ROS (i.e. superoxide) and longevity (Guarente cited above). This could be of high importance, especially in old cells, which suffer from damaged and inefficient mitochondria and therefore generate high ROS levels when respiration is active. In support of this hypothesis, we demonstrate that spermidine-treated cells, which showed largely hypoacetylated histones, reduced their oxygen consumption (FIG. 30, day 20). Intriguingly, these cells resemble the status of quiescence as we demonstrated by sucrose-gradient separation of upper (non-quiescent) and lower (quiescent) cells. Quiescent cells are unbudded cells, exhibiting low ROS, reduced markers of apoptosis and necrosis, and low metabolic rates (Allen et al. (2006) J Cell Biol 174, 89-100).

Interestingly, TOR depletion or rapamycin treatment, which also induces autophagy, similarly causes cells to enter a quiescent (G0-like) state (Jacinto & Hall (2003) Nat Rev Mol Cell Biol 4, 117-126). Thus, autophagic processes as well as hypoacetylation-induced silencing might cooperate to promote longevity of non-growing cells by promoting a low-metabolic quiescent state.

Aging-associated necrotic death can be inhibited by simple spermidine application or by genetic modification of the HAT machinery in yeast, arguing in favor of programmed rather than accidental necrotic death. Necrotic cell death culminates in the leakage of intracellular compounds and consequent local inflammation, which in turn is suspected to be a driving force of aging (“inflammaging”). Recently, Franceschi et al. proposed that chronic inflammation may be one of the driving forces of human aging, causing immunosenescence (C. Franceschi et al. (2007) Mech Ageing Dev 128, 92-105). Thus, programmed necrotic processes might be of cardinal importance to understand the mechanisms of organismal aging in general.

Interestingly, polyamine concentrations decline during aging of various organisms, including humans (Scalabrino & Ferioli (1984) Mech Ageing Dev 26, 149-164) and plants (Kaur-Sawhney et al. (1982) Plant Physiol 69, 405-410), and external application of spermidine inhibits oat leaf senescence (Altman at al. (1977) Plant Physiol 60, 570-574). Moreover, anti-oxidant as well as anti-inflammatory activities of polyamines have been described in human cells (Lovaas & Carlin (1991), Free Radic Biol Med 11, 455-461).

Thus, our findings may have implications ranging from basic aging processes to human aging research.

TABLE 2
Effects on chronological aging of single disruption
of genes involved in histone (de)acetylation.

| Survival during | Pro-survival effect | |
|---|---|---|
| chronological | of spermidine | |
| aging (compared | application | |
| to WT) | (compared to WT) | Single deletion of . . . |
| increased during | reduced | [symbol] |
| early aging (day | | |
| 5 to 15) | | |
| strongly reduced | increased during | [symbol] HDA2 |
| | early aging (due | |
| | to fast death of | |
| | control cultures), | |
| | BUT diminished or | |
| | absent at later | |
| | time points (day | |
| | 15 to 25) | |
| not affected | slightly reduced | [symbol] |
| | during early aging | |
| | (day 5 to 15) | |
| slightly increased | not affected | HDA1 |
| slightly reduced | not affected | [symbol] RXT2, |
| | | SDS3, SAP30 |
| not affected | not affected | [symbol] |
| | | HDA3, HOS1, HOS2, |
| | | HOS3, HOS4, |
| | | RPD3, PHO23, |
| | | SIR2, HST1, |
| | | HST2, SET3, |
| | | SIF2 |

The effects on chronological aging of 28 single deletion strains of genes involved in histone acetylation (bold italic characters) or deacetylation (italic characters) are presented. All strains were aged with and without application of 4 mM spermidine and survival was determined by clonogenicity. Deletion strains were assigned to one of five categories, depending on the effects on survival during aging and the ability of spermidine to improve this survival.
¹The pro-survival effect of spermidine in the [symbol] deleted strain was only reduced until day 10 of aging.

### Example 11

### Effects of Polyamines on Autophagy and Mitochondrial Function in Mammals

The expression level and/or activity of a set of representative proteins involved in autophagy, and mitochondrial enzyme complex activity in liver are determined, at different time points after application of an injury leading to a critically ill condition and administration of the polyamine spermidine.

Electron microscopy pictures of tissue samples are taken to determine changes in the morphology of mitochondria and to determine the number of autophagosomes to assess clearance of less functional or damaged mitochondria.

### Example 12

### Proof-of-Concept Study

The results of the above shown rabbit experiments are confirmed in a statistically well-powered proof-of-concept outcome study. Based on the pilot study, two doses of spermidine are selected (30 and 100 mg/day) to continue with. In order to detect a 25% survival benefit compared to placebo (saline administration), with an alpha level of 0.05 and a power of 0.80, and after correction for multiple comparisons, 58 animals were included per group.

The study was again carried out blinded and performed as outlined in examples 1 to 5. An interim analysis was performed after having included 15 animals per group, for selecting 1 spermidine-group that will be completed next to the saline-group until 58 animals per group are included. After the interim analysis, the codes of the vials are changed, so that the proceeding of the study is again carried out blinded.

The interim analysis confirmed the beneficial results on mortality. Whereas the mortality was high in the placebo-group (saline), the mortality was much lower by administrating spermidine. Both doses had an equal effect on mortality. The lowest dose (30 mg/day) for the continuation of the proof-of-concept study.

Plasma and tissue analyses have been performed on the animals that were included up to the interim analysis. Administration of spermidine 30 mg/d or spermidine 100 mg/d could maintain or increase the plasma spermidine concentration to similar (supra)physiological levels as in the pilot study. Interestingly, in sick rabbits receiving placebo (saline), the plasma spermidine levels were lower in rabbits who did not survive their illness, compared to survivors. Simultaneously, spermidine degradation products were increased in non-survivors. These findings support the hypothesis of the present invention of increased spermidine catabolism during critical illness, which could ultimately lead to a life-threatening spermidine deficiency.

Plasma markers of kidney and liver function were determined the rabbits used in the present experiment. The kidney function appeared protected by spermidine supplementation. Urea and creatinine, two markers of kidney dysfunction that accumulate when renal function decreases, were measured. In the present animal model of critical illness, kidney function spontaneously deteriorates severely over time, and this could be attenuated by spermidine administration. Indeed, the number of animals that developed a more than 3-fold rise in normal creatinine levels dropped from 25% in the saline-treated animals to 2.8% in the spermidine-treated animals (P 0.0161). Likewise, the number of animals that developed a more than 3-fold rise in normal urea levels dropped from 18.8% in the saline-treated animals to 2.8% in the spermidine-treated animals (P 0.0461).

Both kidney and liver of critically ill animals in the saline group showed signs of deficient autophagy. Both tissues of these animals displayed a marked accumulation of the autophagic flux marker p62, a protein that is normally degraded by autophagy and that accumulates in conditions of insufficient autophagy. Compared to saline-treated animals, the rise in renal p62 levels was attenuated by spermidine treatment, which points to a stimulation of autophagy by spermidine in the kidney. The stimulation of autophagy by spermidine in kidney was accompanied by a protection of the renal function. p62 accumulation appeared also important in the determination of liver function, as hepatic p62-levels showed a strong positive correlation with ALT and to a lesser extent with AST, both markers of liver dysfunction. Hence, these analyses corroborate the hypothesis of insufficient autophagy during critical illness as a contributor to organ failure and risk of death. The latter is further illustrated by the more pronounced p62 accumulation in kidney and liver of non-surviving animals, compared to animals that survived their illness. Hence, these findings provide a rationale for autophagy stimulation during critical illness.

All patents, patent application, and publications mentioned in this specification are herein incorporated by reference to the same extent as if each independent patent, patent application, or publication was specifically and individually indicated to be incorporated by reference.

What is claimed is:

1. A method of treating a life threatening condition in a critically ill human patient with a non-infectuous disorder, wherein the critically ill patient is a patient receiving enteral or parenteral nutrition, the method comprising the step of administering to said patient an autophagy inducing agent.

2. The method according to claim 1, wherein the autophagy inducing agent is polyamine or a salt, solvate, or derivative thereof.

3. The method according to claim 2, wherein the polyamine is a metabolisable polyamine.

4. The method according to claim 2, wherein the polyamine is a substrate for the enzyme Spermine/Spermidine Acetyltranferase (SSAT).

5. The method according to claim 2, wherein the polyamine is not modified at one or more of the NH₂ or NH groups.

6. The method according to claim 2, wherein the polyamine is selected from the group consisting of putrescine (1,4-diamino-butane), 1,3-diamino-propane, 1,7-diamino-heptane, 1,8-diamino-octane, spermine, spermidine, cholesteryl spermine, spermidine trihydrochloride, spermidine phosphate hexahydrate, spermidine phosphate hexahydrate, L-arginyl-3,4-spermidine and 1,4-butanediamine N-(3-aminopropyl)-monohydrochloride.

7. The method according to claim 2, wherein the polyamine is spermine or spermidine.

8. The method according to claim 1, wherein the autophagy inducing agent is a component stimulating the mTOR pathway.

9. The method according to claim 8, wherein said component is selected from the group consisting of rapamycin, trehalose, resveratrol, and nicotinamide.

10. The method according to claim 1, wherein the life threatening condition is selected from the group consisting of lactic acidosis, muscle weakening, hyperglycemia, multiple organ failure and failed or disturbed homeostasis.

11. The method according to claim 1 wherein the life threatening condition in said critically ill patients is caused or enhanced by unbalanced parenteral nutrition or a parenteral nutrient delivery that creates nutrient overload.

12. The method according to claim 2, wherein the polyamine is administered together with an enteral or parenteral nutritient composition.

13. The method according to claim 1, wherein the disorder of the critically ill patient is selected from the group consisting of severe or multiple trauma, high risk or extensive surgery, cerebral trauma or bleeding, respiratory insufficiency, abdominal peritonitis, acute kidney injury, acute liver injury, severe burns and critical illness polyneuropathy.

14. The method according to claim 12, wherein said nutritient composition comprises a saccharide.

15. The method according to claim 14, wherein said saccharide is present in said solution in a concentration between 10 and 20% (w/v).

16. The method according to claim 14, wherein said saccharide is glucose.

17. The method according to claim 12, wherein said polyamine is present in said solution in a concentration between 0.05 to 4 (w/v), between 0.5 to 2 (w/v), or between 1 to 1.5% (w/v).
